SEQUENCE LISTING (86 sequences)

SEQ ID NO: 1 - protein, 5 aa, Artificial Sequence
  Synthetic Construct
SYAMH

SEQ ID NO: 2 - protein, 17 aa, Artificial Sequence
  Synthetic Construct
GISSNGGSTYYANSVKG

SEQ ID NO: 3 - protein, 17 aa, Artificial Sequence
  Synthetic Construct
GISPSGSSTYYANSVKG

SEQ ID NO: 4 - protein, 17 aa, Artificial Sequence
  Synthetic Construct
GISPSGVSTYYANSVKG

SEQ ID NO: 5 - protein, 17 aa, Artificial Sequence
  Synthetic Construct
GISPTSGSTYYANSVKG

SEQ ID NO: 6 - protein, 17 aa, Artificial Sequence
  Synthetic Construct
GISPTGTSTYYANSVKG

SEQ ID NO: 7 - protein, 17 aa, Artificial Sequence
  Synthetic Construct
GISSSGSSTYYANSVKG

SEQ ID NO: 8 - protein, 17 aa, Artificial Sequence
  Synthetic Construct
GISPSSTSTYYANSVKG

SEQ ID NO: 9 - protein, 17 aa, Artificial Sequence
  Synthetic Construct
GISPSSASTYYANSVKG

SEQ ID NO: 10 - protein, 17 aa, Artificial Sequence
  Synthetic Construct
GISGNSASTYYANSVKG

SEQ ID NO: 11 - protein, 17 aa, Artificial Sequence
  Synthetic Construct
GISHSGTSTYYANSVKG

SEQ ID NO: 12 - protein, 17 aa, Artificial Sequence
  Synthetic Construct
GISPTSASTYYANSVKG

SEQ ID NO: 13 - protein, 17 aa, Artificial Sequence
  Synthetic Construct
GISPTGGSTYYANSVKG

SEQ ID NO: 14 - protein, 17 aa, Artificial Sequence
  Synthetic Construct
GISHSGNSTYYANSVKG

SEQ ID NO: 15 - protein, 17 aa, Artificial Sequence
  Synthetic Construct
GISPSSNSTYYANSVKG

SEQ ID NO: 16 - protein, 17 aa, Artificial Sequence
  Synthetic Construct
GISYSSASTYYANSVKG

SEQ ID NO: 17 - protein, 11 aa, Artificial Sequence
  Synthetic Construct
VKVGYRGGMDV

SEQ ID NO: 18 - protein, 11 aa, Artificial Sequence
  Synthetic Construct
SKVRYRGGMDV

SEQ ID NO: 19 - protein, 11 aa, Artificial Sequence
  Synthetic Construct
VKVKYRGGMDV

SEQ ID NO: 20 - protein, 11 aa, Artificial Sequence
  Synthetic Construct
VKVRYRGGMDV

SEQ ID NO: 21 - protein, 11 aa, Artificial Sequence
  Synthetic Construct
VKGAYRGGMDV

SEQ ID NO: 22 - protein, 11 aa, Artificial Sequence
  Synthetic Construct
VKVAYRGGMDV

SEQ ID NO: 23 - protein, 11 aa, Artificial Sequence
  Synthetic Construct
VKVLYRGGMDV

SEQ ID NO: 24 - protein, 11 aa, Artificial Sequence
  Synthetic Construct
VKSKYRGGMDV

SEQ ID NO: 25 - protein, 11 aa, Artificial Sequence
  Synthetic Construct
VKLKYRGGMDV

SEQ ID NO: 26 - protein, 11 aa, Artificial Sequence
  Synthetic Construct
VRVLYRGGMDV

SEQ ID NO: 27 - protein, 11 aa, Artificial Sequence
  Synthetic Construct
VFVRYRGGMDV

SEQ ID NO: 28 - protein, 11 aa, Artificial Sequence
  Synthetic Construct
VKGRYRGGMDV

SEQ ID NO: 29 - protein, 11 aa, Artificial Sequence
  Synthetic Construct
VKRRYRGGMDV

SEQ ID NO: 30 - protein, 11 aa, Artificial Sequence
  Synthetic Construct
VKPAYRGGMDV

SEQ ID NO: 31 - protein, 12 aa, Artificial Sequence
  Synthetic Construct
RASQSVSSAYLA

SEQ ID NO: 32 - protein, 12 aa, Artificial Sequence
  Synthetic Construct
RASQGISSAYLA

SEQ ID NO: 33 - protein, 12 aa, Artificial Sequence
  Synthetic Construct
RASQSISTAYLA

SEQ ID NO: 34 - protein, 12 aa, Artificial Sequence
  Synthetic Construct
RASQDISSAYLA

SEQ ID NO: 35 - protein, 12 aa, Artificial Sequence
  Synthetic Construct
RASQDVSSAYLA

SEQ ID NO: 36 - protein, 12 aa, Artificial Sequence
  Synthetic Construct
RASQNISTAYLA

SEQ ID NO: 37 - protein, 12 aa, Artificial Sequence
  Synthetic Construct
RASQDASNAYLA

SEQ ID NO: 38 - protein, 12 aa, Artificial Sequence
  Synthetic Construct
RASQGVSSAYLA

SEQ ID NO: 39 - protein, 12 aa, Artificial Sequence
  Synthetic Construct
RASQSVSTAYLA

SEQ ID NO: 40 - protein, 12 aa, Artificial Sequence
  Synthetic Construct
RASQGVSTAYLA

SEQ ID NO: 41 - protein, 7 aa, Artificial Sequence
  Synthetic Construct
GTSRRAT

SEQ ID NO: 42 - protein, 9 aa, Artificial Sequence
  Synthetic Construct
QLYGSSSVT

SEQ ID NO: 43 - protein, 9 aa, Artificial Sequence
  Synthetic Construct
QLYGATSVT

SEQ ID NO: 44 - protein, 9 aa, Artificial Sequence
  Synthetic Construct
QLYGASSVT

SEQ ID NO: 45 - protein, 9 aa, Artificial Sequence
  Synthetic Construct
QLYGRSSVT

SEQ ID NO: 46 - protein, 9 aa, Artificial Sequence
  Synthetic Construct
QLYGTSSVT

SEQ ID NO: 47 - protein, 9 aa, Artificial Sequence
  Synthetic Construct
QLYGSTSVT

SEQ ID NO: 48 - protein, 120 aa, Artificial Sequence
  Synthetic Construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 49 - protein, 120 aa, Artificial Sequence
  Synthetic Construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 50 - protein, 120 aa, Artificial Sequence
  Synthetic Construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 51 - protein, 120 aa, Artificial Sequence
  Synthetic Construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 52 - protein, 120 aa, Artificial Sequence
  Synthetic Construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 53 - protein, 120 aa, Artificial Sequence
  Synthetic Construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 54 - protein, 120 aa, Artificial Sequence
  Synthetic Construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 55 - protein, 120 aa, Artificial Sequence
  Synthetic Construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 56 - protein, 120 aa, Artificial Sequence
  Synthetic Construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 57 - protein, 120 aa, Artificial Sequence
  Synthetic Construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 58 - protein, 120 aa, Artificial Sequence
  Synthetic Construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 59 - protein, 120 aa, Artificial Sequence
  Synthetic Construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 60 - protein, 120 aa, Artificial Sequence
  Synthetic Construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 61 - protein, 120 aa, Artificial Sequence
  Synthetic Construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 62 - protein, 120 aa, Artificial Sequence
  Synthetic Construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 63 - protein, 120 aa, Artificial Sequence
  Synthetic Construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 64 - protein, 120 aa, Artificial Sequence
  Synthetic Construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 65 - protein, 108 aa, Artificial Sequence
  Synthetic Construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 66 - protein, 108 aa, Artificial Sequence
  Synthetic Construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 67 - protein, 108 aa, Artificial Sequence
  Synthetic Construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 68 - protein, 108 aa, Artificial Sequence
  Synthetic Construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 69 - protein, 108 aa, Artificial Sequence
  Synthetic Construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 70 - protein, 108 aa, Artificial Sequence
  Synthetic Construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 71 - protein, 108 aa, Artificial Sequence
  Synthetic Construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 72 - protein, 108 aa, Artificial Sequence
  Synthetic Construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 73 - protein, 108 aa, Artificial Sequence
  Synthetic Construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 74 - protein, 108 aa, Artificial Sequence
  Synthetic Construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 75 - protein, 108 aa, Artificial Sequence
  Synthetic Construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 76 - protein, 108 aa, Artificial Sequence
  Synthetic Construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 77 - protein, 108 aa, Artificial Sequence
  Synthetic Construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 78 - protein, 17 aa, Artificial Sequence
  Synthetic Construct
  VARIANT 4: Xaa = Ser, Tyr, Pro, Gly, or His
  VARIANT 5: Xaa = Thr, Ser, or Asn
  VARIANT 6: Xaa = Gly or Ser
  VARIANT 7: Xaa = Thr, Ser, Asn, Val, Gly, or Ala
GISXXXXSTYYANSVKG

SEQ ID NO: 79 - protein, 11 aa, Artificial Sequence
  Synthetic Construct
  VARIANT 1: Xaa = Ser or Val
  VARIANT 2: Xaa = Lys, Phe, or Arg
  VARIANT 3: Xaa = Ser, Pro, Val, Gly, Leu, or Arg
  VARIANT 4: Xaa = Gly, Leu, Arg, Ala, or Lys
XXXXYRGGMDV

SEQ ID NO: 80 - protein, 12 aa, Artificial Sequence
  Synthetic Construct
  VARIANT 5: Xaa = Gly, Ser, Asp, or Asn
  VARIANT 6: Xaa = Ala, Ile, or Val
  VARIANT 8: Xaa = Thr, Ser, or Asn
RASQXXSXAYLA

SEQ ID NO: 81 - protein, 9 aa, Artificial Sequence
  Synthetic Construct
  VARIANT 5: Xaa = Ala, Ser, Thr, or Arg
  VARIANT 6: Xaa = Thr or Ser
QLYGXXSVT

SEQ ID NO: 82 - protein, 207 aa, Artificial Sequence
  Synthetic Construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 83 - protein, 825 aa, Artificial Sequence
  Synthetic Construct
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 84 - protein, 330 aa, Artificial Sequence
  Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 107 aa, Artificial Sequence [sequence omitted]

(19) United States
(12) Patent Application Publication
(10) Pub. No.: US 2023/0374144 A1
(43) Pub. Date: Nov. 23, 2023

(54) ANTIBODIES SPECIFICALLY RECOGNIZING INTERLEUKIN-4 RECEPTOR ALPHA AND USES THEREOF

(71) Applicant: STAIDSON (BEIJING) BIOPHARMACEUTICAL CO., LTD., Beijing (CN)

(72) Inventors: Zhiliang CAO, Beijing (CN); Debin YU, Beijing (CN); Shuping ZHANG, Beijing (CN)

(73) Assignee: STAIDSON (BEIJING) BIOPHARMACEUTICAL CO., LTD., Beijing (CN)

(21) Appl. No.: 18/044,703

(22) PCT Filed: Sep. 9, 2021
(86) PCT No.: PCT/CN2021/117366

§ 371 Date: Mar. 9, 2023

(30) Foreign Application Priority Data
Sep. 10, 2020 (WO) PCT/CN2020/114406

(51) Int. Cl. C07K 16/28

(52) U.S. Cl. CPC C07K 16/2866; C07K 2317/92; C07K 2317/76; C07K 2317/73; C07K 2317/21

(57) ABSTRACT

Provided are antibodies including antigen binding fragment thereof that specifically recognizing Interleukin-4 receptor α (IL-4Rα). Also provided are methods of making and using these antibodies.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 202008141640_SEQLIST-IL4R.txt, date recorded: Aug. 14, 2020, size: 57.5 KB).

### FIELD OF THE APPLICATION

The present application pertains to antibodies that specifically recognize interleukin-4 receptor alpha (IL-4Ra), and methods of manufacture and uses thereof, including methods of treating one or more diseases or disorders which are caused by increased expression, activity, or sensitivity of human interleukin-4 (hIL-4) and/or human interleukin-13 (hIL-13) and/or human interleukin-4 receptor alpha (hIL-4Ra).

### BACKGROUND OF THE APPLICATION

Interleukin-4 (IL-4) and Interleukin-13 (IL-13) are significant cytokines related to the type II inflammatory response. They play important roles in regulating the responses of lymphocytes, myeloid cells, and non-hematopoietic cells. The cytokine-binding receptor chain for IL-4 is IL-4Rα, which is widely expressed in various types of cells. Upon IL-4 binding to IL-4Rα, the IL-4/IL-4Rα-complex will bind a secondary receptor chain, either IL-2Rγc (γc) or IL-13Rα1. The expression of these secondary chains varies among different cell types. In non-hematopoietic cells, γc expression is low or absent, whereas higher amounts of IL-13Rα1 are expressed in these cells. By contrast, lymphocytes express only low levels of IL-13Rα1 and relatively large amounts of γc. Finally, myeloid cells fall in between non-hematopoietic cells and lymphocytes, as they express both IL-13Rα1 and γc. Antibodies against human IL-4Rα are described in U.S. Pat. Nos. 5,717,072, 7,186,809 and 7,605,237.

The disclosures of all publications, patents, patent applications and published patent applications referred to herein are hereby incorporated herein by reference in their entirety.

### BRIEF SUMMARY OF THE APPLICATION

In one aspect, the present application provides an isolated anti-IL-4Rα antibody that specifically binds to human IL-4Rα. In some embodiments, the isolated anti-IL-4Rα antibody binds to the human IL-4Rα with a Kd from about 0.1 pM to about 10 nM. In some embodiments, the present application provides an isolated anti-IL-4Rα antibody comprising: a heavy chain variable domain (V_{H}) comprising a heavy chain complementarity determining region (HC-CDR) 1 comprising SYAMH (SEQ ID NO: 1); an HC-CDR2 comprising GISX₁X₂X₃X₄STYYANSVKG (SEQ ID NO: 78), wherein X₁ is P, S, H, G, or Y, X₂ is S, T, or N, X₃ is G or S, X₄ is 5, V, G, T, A, or N; and an HC-CDR3 comprising X₁X₂X₃X₄YRGGMDV (SEQ ID NO: 79), wherein X₁ is V or S, X₂ is K, F, or R, X₃ is P, V, G, R, S, or L, X₄ is G, A, R, K, or L; and a light chain variable domain (V_{L}) comprising a light chain complementarity determining region (LC-CDR) 1 comprising RASQX₁X₂SX₃AYLA (SEQ ID NO: 80), wherein X₁ is G, S, N, or D, X₂ is I, V, or A, X₃ is S, T, or N; an LC-CDR2 comprising GTSRRAT (SEQ ID NO: 41); and an LC-CDR3 comprising QLYGX₁X₂SVT (SEQ ID NO: 81), wherein X₁ is A, S, T, or R, X₂ is T or S.

In some embodiments, there is provided an isolated anti-IL-4Rα antibody comprising: a V_{H} comprising an HC-CDR1 comprising the amino acid sequence of SEQ ID NO: 1, or a variant thereof comprising up to about 3 amino acid substitutions; an HC-CDR2 comprising the amino acid sequence of any one of SEQ ID NOs: 2-16, or a variant thereof comprising up to about 3 amino acid substitutions; and an HC-CDR3 comprising the amino acid sequence of any one of SEQ ID NOs: 17-30, or a variant thereof comprising up to about 3 amino acid substitutions; and a V_{L} comprising an LC-CDR1 comprising the amino acid sequence of any one of SEQ ID NOs: 31-40, or a variant thereof comprising up to about 3 amino acid substitutions; an LC-CDR2 comprising the amino acid sequence of SEQ ID NO: 41, or a variant thereof comprising up to about 3 amino acid substitutions; and an LC-CDR3 comprising the amino acid sequence of any one of SEQ ID NOs: 42-47, or a variant thereof comprising up to about 3 amino acid substitutions.

In some embodiments, there is provided an isolated anti-IL-4Rα antibody comprising a V_{H} comprising an HC-CDR1, an HC-CDR2, and an HC-CDR3 of a V_{H} comprising the amino acid sequence of any one of SEQ ID NOs: 48-64; and a V_{L} comprising an LC-CDR1, an LC-CDR2, and an LC-CDR3 of a V_{L} comprising the amino acid sequence of any one of SEQ ID NOs: 65-77.

In some embodiments, there is provided an isolated anti-IL-4Rα antibody comprising: (i) a V_{H} comprising an HC-CDR1, an HC-CDR2, and an HC-CDR3 of a V_{H} comprising the amino acid sequence of SEQ ID NO: 48; and a V_{L} comprising an LC-CDR1, an LC-CDR2, and an LC-CDR3 of a V_{L} comprising the amino acid sequence of SEQ ID NO: 65; (ii) a V_{H} comprising an HC-CDR1, an HC-CDR2, and an HC-CDR3 of a V_{H} comprising the amino acid sequence of SEQ ID NO: 49; and a V_{L} comprising an LC-CDR1, an LC-CDR2, and an LC-CDR3 of a V_{L} comprising the amino acid sequence of SEQ ID NO: 66; (iii) a V_{H} comprising an HC-CDR1, an HC-CDR2, and an HC-CDR3 of a V_{H} comprising the amino acid sequence of SEQ ID NO: 50; and a V_{L} comprising an LC-CDR1, an LC-CDR2, and an LC-CDR3 of a V_{L} comprising the amino acid sequence of SEQ ID NO: 66; (iv) a V_{H} comprising an HC-CDR1, an HC-CDR2, and an HC-CDR3 of a V_{H} comprising the amino acid sequence of SEQ ID NO: 51; and a V_{L} comprising an LC-CDR1, an LC-CDR2, and an LC-CDR3 of a V_{L} comprising the amino acid sequence of SEQ ID NO: 67; (v) a V_{H} comprising an HC-CDR1, an HC-CDR2, and an HC-CDR3 of a V_{H} comprising the amino acid sequence of SEQ ID NO: 52; and a V_{L} comprising an LC-CDR1, an LC-CDR2, and an LC-CDR3 of a V_{L} comprising the amino acid sequence of SEQ ID NO: 67; (vi) a V_{H} comprising an HC-CDR1, an HC-CDR2, and an HC-CDR3 of a V_{H} comprising the amino acid sequence of SEQ ID NO: 53; and a V_{L} comprising an LC-CDR1, an LC-CDR2, and an LC-CDR3 of a V_{L} comprising the amino acid sequence of SEQ ID NO: 66; (vii) a V_{H} comprising an HC-CDR1, an HC-CDR2, and an HC-CDR3 of a V_{H} comprising the amino acid sequence of SEQ ID NO: 54; and a V_{L} comprising an LC-CDR1, an LC-CDR2, and an LC-CDR3 of a V_{L} comprising the amino acid sequence of SEQ ID NO: 67; (viii) a V_{H} comprising an HC-CDR1, an HC-CDR2, and an HC-CDR3 of a V_{H} comprising the amino acid sequence of SEQ ID NO: 55; and a V_{L} comprising an LC-CDR1, an LC-CDR2, and an LC-CDR3 of a V_{L} comprising the amino acid sequence of SEQ ID NO: 68; (ix) a V_{H} comprising an HC-CDR1, an HC-CDR2, and an HC-CDR3 of a V_{H} comprising the amino acid sequence of SEQ ID NO: 56; and a V_{L} comprising an LC-CDR1, an LC-CDR2, and an LC-CDR3 of a V_{L} comprising the amino acid sequence of SEQ ID NO: 69; (x) a V_{H} comprising an HC-CDR1, an HC-CDR2, and an HC-CDR3 of a V_{H} comprising the amino acid sequence of SEQ ID NO: 57; and a V_{L} comprising an LC-CDR1, an LC-CDR2, and an LC-CDR3 of a V_{L} comprising the amino acid sequence of SEQ ID NO: 70; (xi) a V_{H} comprising an HC-CDR1, an HC-CDR2, and an HC-CDR3 of a V_{H} comprising the amino acid sequence of SEQ ID NO: 50; and a V_{L} comprising an LC-CDR1, an LC-CDR2, and an LC-CDR3 of a V_{L} comprising the amino acid sequence of SEQ ID NO: 71; (xii) a V_{H} comprising an HC-CDR1, an HC-CDR2, and an HC-CDR3 of a V_{H} comprising the amino acid sequence of SEQ ID NO: 58; and a V_{L} comprising an LC-CDR1, an LC-CDR2, and an LC-CDR3 of a V_{L} comprising the amino acid sequence of SEQ ID NO: 72; (xiii) a V_{H} comprising an HC-CDR1, an HC-CDR2, and an HC-CDR3 of a V_{H} comprising the amino acid sequence of SEQ ID NO: 59; and a V_{L} comprising an LC-CDR1, an LC-CDR2, and an LC-CDR3 of a V_{L} comprising the amino acid sequence of SEQ ID NO: 73; (xiv) a V_{H} comprising an HC-CDR1, an HC-CDR2, and an HC-CDR3 of a V_{H} comprising the amino acid sequence of SEQ ID NO: 60; and a V_{L} comprising an LC-CDR1, an LC-CDR2, and an LC-CDR3 of a V_{L} comprising the amino acid sequence of SEQ ID NO: 74; (xv) a V_{H} comprising an HC-CDR1, an HC-CDR2, and an HC-CDR3 of a V_{H} comprising the amino acid sequence of SEQ ID NO: 61; and a V_{L} comprising an LC-CDR1, an LC-CDR2, and an LC-CDR3 of a V_{L} comprising the amino acid sequence of SEQ ID NO: 75; (xvi) a V_{H} comprising an HC-CDR1, an HC-CDR2, and an HC-CDR3 of a V_{H} comprising the amino acid sequence of SEQ ID NO: 62; and a V_{L} comprising an LC-CDR1, an LC-CDR2, and an LC-CDR3 of a V_{L} comprising the amino acid sequence of SEQ ID NO: 69; (xvii) a V_{H} comprising an HC-CDR1, an HC-CDR2, and an HC-CDR3 of a V_{H} comprising the amino acid sequence of SEQ ID NO: 63; and a V_{L} comprising an LC-CDR1, an LC-CDR2, and an LC-CDR3 of a V_{L} comprising the amino acid sequence of SEQ ID NO: 76; or (xviii) a V_{H} comprising an HC-CDR1, an HC-CDR2, and an HC-CDR3 of a V_{H} comprising the amino acid sequence of SEQ ID NO: 64; and a V_{L} comprising an LC-CDR1, an LC-CDR2, and an LC-CDR3 of a V_{L} comprising the amino acid sequence of SEQ ID NO: 77.

In some embodiments, the isolated anti-IL-4Rα antibody binds to human IL-4Rα with a Kd from about 0.1 pM to about 10 nM.

In some embodiments, there is provided an isolated anti-IL-4Rα antibody comprising: (i) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs; (ii) a V_{H} comprising an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (iii) a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (iv) a V_{H} comprising an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs; (v) a V_{H} comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs; (vi) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (vii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs; (viii) a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24, or a variant thereof comprising up to about 5 amino acid substitutions in the HC-CDRs; and a V_{L} comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs; (ix) a V_{H} comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25, or a variant thereof comprising up to about 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (x) a V_{H} comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26, or a variant thereof comprising up to about 5 amino acid substitutions in the HC-CDRs; and a V_{L} comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (xi) a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (xii) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27, or a variant thereof comprising up to about 5 amino acid substitutions in the HC-CDRs; and a V_{L} comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs; (xiii) a V_{H} comprising an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45, or a variant thereof comprising up to about 5 amino acid substitutions in the LC-CDRs; (xiv) a V_{H} comprising an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46, or a variant thereof comprising up to about 5 amino acid substitutions in the LC-CDRs; (xv) a V_{H} comprising an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29, or a variant thereof comprising up to about 5 amino acid substitutions in the HC-CDRs; and a V_{L} comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (xvi) a V_{H} comprising an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (xvii) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30, or a variant thereof comprising up to about 5 amino acid substitutions in the HC-CDRs; and a V_{L} comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; (xviii) a V_{H} comprising an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47, or a variant thereof comprising up to about 5 amino acid substitutions in the LC-CDRs.

In some embodiments, according to any one of the isolated anti-IL-4Rα antibodies described above, the isolated anti-IL-4Rα antibody comprises: a V_{H} comprising the amino acid sequence of any one of SEQ ID NOs: 48-64, or a variant thereof having at least about 90% sequence identity to the amino acid sequence of any one of SEQ ID NOs: 48-64; and a V_{L} comprising the amino acid sequence of any one of SEQ ID NOs: 65-77, or a variant thereof having at least about 90% sequence identity to the amino acid sequence of any one of SEQ ID NOs: 65-77.

In some embodiments, the isolated anti-IL-4Rα antibody comprises: (i) a V_{H} comprising the amino acid sequence of SEQ ID NO: 48, or a variant thereof having at least about 90% sequence identity to the amino acid sequence of SEQ ID NO: 48; and a V_{L} comprising the amino acid sequence of SEQ ID NO: 65, or a variant thereof having at least about 90% sequence identity to the amino acid sequence of SEQ ID NO: 65; (ii) a V_{H} comprising the amino acid sequence of SEQ ID NO: 49, or a variant thereof having at least about 90% sequence identity to the amino acid sequence of SEQ ID NO: 49; and a V_{L} comprising the amino acid sequence of SEQ ID NO: 66, or a variant thereof having at least about 90% sequence identity to the amino acid sequence of SEQ ID NO: 66; (iii) a V_{H} comprising the amino acid sequence of SEQ ID NO: 50, or a variant thereof having at least about 90% sequence identity to the amino acid sequence of SEQ ID NO: 50; and a V_{L} comprising the amino acid sequence of SEQ ID NO: 66, or a variant thereof having at least about 90% sequence identity to the amino acid sequence of SEQ ID NO: 66; (iv) a V_{H} comprising the amino acid sequence of SEQ ID NO: 51, or a variant thereof having at least about 90% sequence identity to the amino acid sequence of SEQ ID NO: 51; and a V_{L} comprising the amino acid sequence of SEQ ID NO: 67, or a variant thereof having at least about 90% sequence identity to the amino acid sequence of SEQ ID NO: 67; (v) a V_{H} comprising the amino acid sequence of SEQ ID NO: 52, or a variant thereof having at least about 90% sequence identity to the amino acid sequence of SEQ ID NO: 52; and a V_{L} comprising the amino acid sequence of SEQ ID NO: 67, or a variant thereof having at least about 90% sequence identity to the amino acid sequence of SEQ ID NO: 67; (vi) a V_{H} comprising the amino acid sequence of SEQ ID NO: 53, or a variant thereof having at least about 90% sequence identity to the amino acid sequence of SEQ ID NO: 53; and a V_{L} comprising the amino acid sequence of SEQ ID NO: 66, or a variant thereof having at least about 90% sequence identity to the amino acid sequence of SEQ ID NO: 66; (vii) a V_{H} comprising the amino acid sequence of SEQ ID NO: 54, or a variant thereof having at least about 90% sequence identity to the amino acid sequence of SEQ ID NO: 54; and a V_{L} comprising the amino acid sequence of SEQ ID NO: 67, or a variant thereof having at least about 90% sequence identity to the amino acid sequence of SEQ ID NO: 67; (viii) a V_{H} comprising the amino acid sequence of SEQ ID NO: 55, or a variant thereof having at least about 90% sequence identity to the amino acid sequence of SEQ ID NO: 55; and a V_{L} comprising the amino acid sequence of SEQ ID NO: 68, or a variant thereof having at least about 90% sequence identity to the amino acid sequence of SEQ ID NO: 68; (ix) a V_{H} comprising the amino acid sequence of SEQ ID NO: 56, or a variant thereof having at least about 90% sequence identity to the amino acid sequence of SEQ ID NO: 56; and a V_{L} comprising the amino acid sequence of SEQ ID NO: 69, or a variant thereof having at least about 90% sequence identity to the amino acid sequence of SEQ ID NO: 69; (x) a V_{H} comprising the amino acid sequence of SEQ ID NO: 57, or a variant thereof having at least about 90% sequence identity to the amino acid sequence of SEQ ID NO: 57; and a V_{L} comprising the amino acid sequence of SEQ ID NO: 70, or a variant thereof having at least about 90% sequence identity to the amino acid sequence of SEQ ID NO: 70; (xi) a V_{H} comprising the amino acid sequence of SEQ ID NO: 50, or a variant thereof having at least about 90% sequence identity to the amino acid sequence of SEQ ID NO: 50; and a V_{L} comprising the amino acid sequence of SEQ ID NO: 71, or a variant thereof having at least about 90% sequence identity to the amino acid sequence of SEQ ID NO: 71; (xii) a V_{H} comprising the amino acid sequence of SEQ ID NO: 58, or a variant thereof having at least about 90% sequence identity to the amino acid sequence of SEQ ID NO: 58; and a V_{L} comprising the amino acid sequence of SEQ ID NO: 72, or a variant thereof having at least about 90% sequence identity to the amino acid sequence of SEQ ID NO: 72; (xiii) a V_{H} comprising the amino acid sequence of SEQ ID NO: 59, or a variant thereof having at least about 90% sequence identity to the amino acid sequence of SEQ ID NO: 59; and a V_{L} comprising the amino acid sequence of SEQ ID NO: 73, or a variant thereof having at least about 90% sequence identity to the amino acid sequence of SEQ ID NO: 73; (xiv) a V_{H} comprising the amino acid sequence of SEQ ID NO: 60, or a variant thereof having at least about 90% sequence identity to the amino acid sequence of SEQ ID NO: 60; and a V_{L} comprising the amino acid sequence of SEQ ID NO: 74, or a variant thereof having at least about 90% sequence identity to the amino acid sequence of SEQ ID NO: 74; (xv) a V_{H} comprising the amino acid sequence of SEQ ID NO: 61, or a variant thereof having at least about 90% sequence identity to the amino acid sequence of SEQ ID NO: 61; and a V_{L} comprising the amino acid sequence of SEQ ID NO: 75, or a variant thereof having at least about 90% sequence identity to the amino acid sequence of SEQ ID NO: 75; (xvi) a V_{H} comprising the amino acid sequence of SEQ ID NO: 62, or a variant thereof having at least about 90% sequence identity to the amino acid sequence of SEQ ID NO: 62; and a V_{L} comprising the amino acid sequence of SEQ ID NO: 69, or a variant thereof having at least about 90% sequence identity to the amino acid sequence of SEQ ID NO: 69; (xvii) a V_{H} comprising the amino acid sequence of SEQ ID NO: 63, or a variant thereof having at least about 90% sequence identity to the amino acid sequence of SEQ ID NO: 63; and a V_{L} comprising the amino acid sequence of SEQ ID NO: 76, or a variant thereof having at least about 90% sequence identity to the amino acid sequence of SEQ ID NO: 76; or (xviii) a V_{H} comprising the amino acid sequence of SEQ ID NO: 64, or a variant thereof having at least about 90% sequence identity to the amino acid sequence of SEQ ID NO: 64; and a V_{L} comprising the amino acid sequence of SEQ ID NO: 77, or a variant thereof having at least about 90% sequence identity to the amino acid sequence of SEQ ID NO: 77.

In some embodiments, there is provided an isolated anti-IL-4Rα antibody that specifically binds to IL-4Rα competitively with any one of the isolated anti-IL-4Rα antibodies described above. In some embodiments, there is provided an isolated anti-IL-4Rα antibody that specifically binds to the same epitope as any one of isolated anti-IL-4Rα antibodies described above.

In some embodiments according to any of the isolated anti-IL-4Rα antibodies described above, the isolated anti-IL-4Rα antibody comprises an Fc fragment. In some embodiments, the isolated anti-IL-4Rα antibody is a full-length IgG antibody. In some embodiments, the isolated anti-IL-4Rα antibody is a full-length IgG1 or IgG4 antibody. In some embodiments, the anti-IL-4Rα antibody is a chimeric, human, or humanized antibody. In some embodiments, the anti-IL-4Rα antibody is an antigen binding fragment selected from the group consisting of a Fab, a Fab′, a F(ab)′2, a Fab′-SH, a single-chain Fv (scFv), an Fv fragment, a dAb, a Fd, a nanobody, a diabody, and a linear antibody.

In some embodiments, the isolated anti-IL-4Rα antibody binds to human IL-4Rα, wherein the anti-IL-4Rα antibody inhibits binding of IL-4 to IL-4Rα, and wherein the anti-IL-4Rα antibody: (i) has an IC50 neutralizing potency of 18 nM or less in a hIL-4R-mediated cellular function inhibition assay in vitro with 1.2 ng/ml of human IL-4; (ii) has an IC50 neutralizing potency of 2.0 nM or less in a hIL-4R-mediated cellular function inhibition assay in vitro with 4 ng/ml of human IL-13; (iii) has an IC50 neutralizing potency of 0.8 nM or less in a TF-1 cell proliferation assay with 2 ng/ml of human IL-4; (iv) has an IC50 neutralizing potency of 0.9 nM or less in a TF-1 cell proliferation assay with 10 ng/ml of human IL-13; (v) has an IC50 neutralizing potency of 1.9 nM or less in a thymus and activation-regulated chemokine (TARC) release assay with 32 ng/ml of human IL-4; (vi) has an IC50 neutralizing potency of 0.1 nM or less in a thymus and activation-regulated chemokine (TARC) release assay with 200 ng/ml of human IL-13; (vii) has an IC50 neutralizing potency of 0.4 nM or less in a CD23 upregulation assay with 1 ng/ml of human IL-4; or (viii) has an IC50 neutralizing potency of 8.4 nM or less in a CD23 upregulation assay with 20 ng/ml of human IL-13.

In some embodiments, there is provided isolated nucleic acid molecule(s) that encodes any one of the anti-IL-4Rα antibodies described above. In some embodiments, there is provided a vector comprising any one of the nucleic acid molecules described above. In some embodiments, there is provided a host cell expressing any one of the anti-IL-4Rα antibodies described above. In some embodiments, there is provided a host cell comprising any one of the nucleic acid molecules described above, or any one of the vectors described above. In some embodiments, there is provided a method of producing an anti-IL-4Rα antibody, comprising: a) culturing any one of the host cells described above under conditions effective to express the anti-IL-4Rα antibody; and b) obtaining the expressed anti-IL-4Rα antibody from the host cell.

In some embodiments, there is provided a method of treating a disease or condition in an individual in need thereof, comprising administering to the individual an effective amount of any one of the anti-IL-4Rα antibodies described above. In some embodiments, provided is the use of any one of the anti-IL-4Rα antibodies described above, or a pharmaceutical composition comprising an anti-IL-4Rα antibody according to any one of the described above in the manufacture of a medicament for treating a disease or condition. In some embodiments, the disease or condition is caused by increased expression, activity or sensitivity of human interleukin-4 (hIL-4) and/or human interleukin-13 (hIL-13) and/or human interleukin-4 receptor alpha (hIL-4Ra). In some embodiments, the disease or condition is selected from the group consisting of asthma, atopic dermatitis, arthritis, herpetiformis (e.g., dermatitis herpetiformis), chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis.

Also provided are pharmaceutical compositions, kits and articles of manufacture comprising any one of the anti-IL-4Rα antibodies described above.

### BRIEF DESCRIPTION OF THE DRAWINGS

FIG. 1 shows the ability of the optimized anti-IL-4Rα antibodies to block human IL-4 binding to human IL-4Rα as analyzed by ELISA.

FIG. 2A shows the ability of the optimized anti-IL4Rα antibodies to neutralize biological effect of human IL-4 using HEK-Blue™ IL-4/IL-13 cells in vitro. FIG. 2B shows the ability of the optimized anti-IL-4Rα antibodies to neutralize biological effect of human IL-13 using HEK-Blue™ IL-4/IL-13 cells in vitro.

FIG. 3A shows the result of the optimized anti-IL4Rα antibodies inhibiting proliferation of TF-1 cells stimulated with human IL-4. FIG. 3B shows the result of the optimized anti-IL-4Rα antibodies inhibiting the proliferation of TF-1 cells stimulated with human IL-13.

FIG. 4A shows the results of the optimized anti-IL4Rα antibodies inhibiting TARC release in human PBMCs stimulated with human IL-4. FIG. 4B shows the results of the optimized anti-IL4Rα antibodies inhibiting TARC release in human PBMCs stimulated with human IL-13.

FIG. 5A shows the results of the optimized anti-IL4Rα antibodies inhibiting human IL-4 dependent CD23 upregulation on B cells in human PBMCs. FIG. 5B shows the results of the optimized anti-IL4Rα antibodies inhibiting human IL-13 dependent CD23 upregulation on B cells in human PBMCs.

### DETAILED DESCRIPTION OF THE APPLICATION

In one aspect, the present application provides anti-IL-4Rα antibodies. By using a combination of selections on naïve scFv phage libraries, affinity maturation, and appropriately designed biochemical and biological assays, the inventors have identified highly potent antibody molecules that bind to human IL-4Rα and inhibit the action of human IL-4 and IL-13 to its receptors. The results presented herein indicate that the antibodies provided by the present application are even more potent and effective than the known and widely used anti-IL-4Rα antibodies Dupilumab (IL-4Rα antibody, Regeneron) and AMG317(IL-4Rα antibody, Amgen, U.S. Pat. No. 8,679,487B) as demonstrated in a variety of biological assays.

The anti-IL-4Rα antibodies provided by the present application include, for example, full-length anti-IL-4Rα antibodies, anti-IL-4Rα scFvs, anti-IL-4Rα Fc fusion proteins, multi-specific (such as bispecific) anti-IL-4Rα antibodies, anti-IL-4Rα immunoconjugates, and the like.

In another aspect, there is provided an anti-IL-4Rα antibody, wherein the anti-IL-4Rα antibody comprises a heavy chain variable domain (V_{H}) comprising an HC-CDR1 comprising SYAMH (SEQ ID NO: 1); an HC-CDR2 comprising GISX₁X₂X₃X₄STYYANSVKG (SEQ ID NO: 78), wherein X₁ is P, S, H, G, or Y, X₂ is S, T, or N, X₃ is G or S, X₄ is 5, V, G, T, A, or N; and an HC-CDR3 comprising X₁X₂X₃X₄YRGGMDV (SEQ ID NO: 79), wherein X₁ is V or S, X₂ is K, F, or R, X₃ is P, V, G, R, S, or L, X₄ is G, A, R, K, or L; and a light chain variable domain (V_{L}) comprising an LC-CDR1 comprising RASQX₁X₂SX₃AYLA (SEQ ID NO: 80), wherein X₁ is G, S, N, or D, X₂ is I, V, or A, X₃ is S, T, or N; an LC-CDR2 comprising GTSRRAT (SEQ ID NO: 41); and an LC-CDR3 comprising QLYGX₁X₂SVT (SEQ ID NO: 81), wherein X₁ is A, S, T, or R, X₂ is T or S.

Also provided are nucleic acids encoding the anti-IL-4Rα antibodies, compositions comprising the anti-IL-4Rα antibodies, and methods of making and using the anti-IL-4Rα antibodies described in the present application.

### Definitions

The term “human IL-4Rα” (hIL-4Ra), as used herein, is intended to refer to a human cytokine receptor that specifically binds interleukin-4 (IL-4). The term “human interleukin-13” (hIL-13) refers to a human cytokine that specifically binds IL-13 receptor, and “hIL-13/hIL-13R1 complex” refers to the complex formed by hIL-13 binding to hIL-13R1, which binds IL-4 receptor alpha to initiate biological activity.

As used herein, “treatment” or “treating” is an approach for obtaining beneficial or desired results, including clinical results. For purposes of the present application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more of other medications required to treat the disease, delaying the progression of the disease, increasing or improving the quality of life, increasing weight gain, and/or prolonging survival. Also encompassed by “treatment” is a reduction of pathological consequence of the disease (such as, for example, tumor volume for cancer). The methods of the application contemplate any one or more of these aspects of treatment.

The term “antibody” includes full-length antibodies and antigen-binding fragments thereof. A full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to different classes or isotypes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein includes an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a single domain antibody, a nanobody, a domain antibody, a bivalent domain antibody, or any other antibody fragments that bind to an antigen but do not comprise a complete antibody structure. An antigen-binding fragment also includes a fusion protein comprising the antibody fragment described above. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two or more antibodies or antibody moieties may bind the same epitope within an antigen if they exhibit competitive binding for the antigen.

As used herein, a first antibody “competes” for binding to a target IL-4Rα with a second antibody when the first antibody inhibits target IL-4Rα binding of the second antibody by at least about 50% (such as at least about any of 55%, 60%, 65%, 70%, 75%, 80%, 85%, 90%, 95%, 98% or 99%) in the presence of an equimolar concentration of the first antibody, or vice versa. A high throughput process for “binning” antibodies based upon their cross-competition is described in PCT Publication No. WO 03/48731.

As used herein, the term “specifically binds,” “specifically recognizing,” or “is specific for” refers to measurable and reproducible interactions, such as binding between a target and an antibody that is determinative of the presence of the target in the presence of a heterogeneous population of molecules, including biological molecules. For example, an antibody that specifically recognizes a target (which can be an epitope) is an antibody that binds to this target with greater affinity, avidity, more readily, and/or with greater duration than its bindings to other targets. In some embodiments, an antibody that specifically recognizes an antigen reacts with one or more antigenic determinants of the antigen with a binding affinity that is at least about 10 times its binding affinity for other targets.

An “isolated” anti-IL-4Rα antibody as used herein refers to an anti-IL-4Rα antibody that (1) is not associated with proteins found in nature, (2) is free of other proteins from the same source, (3) is expressed by a cell from a different species, or, (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR DEFINITIONS

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

The term “chimeric antibody” refers to antibody in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this application (see U.S. Pat. No. 4,816,567; and Morrison et al., Proc. Natl. Acad. Sci. USA, 81:6851-6855 (1984)).

“Fv” is the minimum antibody fragment which contains a complete antigen-recognition and binding site. This fragment consists of a dimer of one heavy- and one light-chain variable region domain in a tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” or “homology” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skilled in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR of the present application is one that binds to an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phe158, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, and rescuing them from a default degradative pathway.

The “CH1 domain” of a human IgG Fc region usually extends from about amino acid 118 to about amino acid 215 (EU numbering system).

“Hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S-S bonds in the same positions.

The “CH2 domain” of a human IgG Fc region usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” comprises the stretch of residues of C-terminal to a CH2 domain in an Fc region (i.e. from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include Clq binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent Kd or IC50 value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent Kd or higher IC50 value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell by cytotoxins. The antibodies “arm” the cytotoxic cells and are required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective in mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5-fold to about 100-fold, e.g. from about 25 to about 50-fold, more effective in mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g. as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-IL-4Rα antibody or composition as disclosed herein, is an amount sufficient to carry out a specifically stated purpose. An “effective amount” can be determined empirically and by known methods relating to the stated purpose.

The term “therapeutically effective amount” refers to an amount of an anti-IL-4Rα antibody or composition as disclosed herein, effective to “treat” a disease or disorder in an individual. In the case of asthma, the “asthma-associated parameters” was used as indicators for evaluating the effect of asthma treatment, for examples, “asthma-associated parameters” include: (a) forced expiratory volume in 1 second (FEV1); (b) peak expiratory flow rate (PEF), including morning PEF (AM PEF) and evening PEF (PM PEF); (c) use of an inhaled bronchodilator such as albuterol or levalbuterol; (d) five-item Asthma Control Questionnaire (ACQ5) score; (d) nighttime awakenings; and (e) 22-item Sino-Nasal Outcome Test (SNOT-22) score. The therapeutically effective amount of the anti-IL-4Rα antibody or composition as disclosed herein can increase one or more of FEV1, AM PEF or PM PEF from baseline, and/or decrease one or more of daily albuterol/levalbuterol use, ACQ5 score, average nighttime awakenings or SNOT-22 score from baseline. As used herein, the term “baseline,” with regard to an asthma-associated parameter, means the numerical value of the asthma-associated parameter for a patient prior to or at the time of administration of a pharmaceutical composition of the present invention. In some embodiments, the improvement in an asthma-associated parameter is an increase of at least 0.10 L from baseline of FEV1. In some embodiments, the improvement in an asthma-associated parameter is an increase of at least 10.0 L/min from baseline of AM PEF. In some embodiments, the improvement in an asthma-associated parameter is an increase of at least 1.0 L/min from baseline of PM PEF. In some embodiments, the improvement in an asthma-associated parameter is a decrease in albuterol/levalbuterol use of at least 1 puff(s) per day from baseline. In some embodiments, the improvement in an asthma-associated parameter is a decrease of at least 0.5 points from baseline in ACQ5 score. In some embodiments, the improvement in an asthma-associated parameter is a decrease of at least 0.2 times per night from baseline of nighttime awakenings. In some embodiments, the improvement in an asthma-associated parameter is a decrease of at least 5 points from baseline in SNOT-22 score. In some embodiments, the therapeutically effective amount is an amount that can adequately control the disease. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biological or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

It is understood that embodiments of the application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein and in the appended claims, the singular forms “a,” “an,” and “the” include plural referents unless the context clearly dictates otherwise.

Anti-IL-4Rα Antibodies

In one aspect, the present application provides anti-IL-4Rα antibodies that specifically bind to IL-4Rα. Anti-IL-4Rα antibodies include, but are not limited to, humanized antibodies, chimeric antibodies, mouse antibodies, human antibodies, and antibodies comprising the heavy chain and/or light chain CDRs discussed herein. In one aspect, the present application provides isolated antibodies that bind to IL-4Rα. Contemplated anti-IL-4Rα antibodies include, for example, full-length anti-IL-4Rα antibodies (e.g., full-length IgG1 or IgG4), anti-IL-4Rα scFvs, anti-IL-4Rα Fc fusion proteins, multi-specific (such as bispecific) anti-IL-4Rα antibodies, anti-IL-4Rα immunoconjugates, and the like. In some embodiments, the anti-IL-4Rα antibody is a full-length antibody (e.g., full-length IgG1 or IgG4) or antigen-binding fragment thereof, which specifically binds to IL-4Rα. In some embodiments, the anti-IL-4Rα antibody is a Fab, a Fab′, a F(ab)′2, a Fab′-SH, a single-chain Fv (scFv), an Fv fragment, a dAb, a Fd, a nanobody, a diabody, or a linear antibody. In some embodiments, reference to an antibody that specifically binds to IL-4Rα means that the antibody binds to IL-4Rα with an affinity that is at least about 10 times (including for example at least about any one of 10, 10², 10³, 10⁴, 10⁵, 10⁶, or 10⁷ times) more tightly than its binding affinity for a non-target. In some embodiments, the non-target is an antigen that is not IL-4Rα. Binding affinity can be determined by methods known in the art, such as ELISA, fluorescence activated cell sorting (FACS) analysis, or radioimmunoprecipitation assay (MA). Kd can be determined by methods known in the art, such as surface plasmon resonance (SPR) assay or biolayer interferometry (BLI).

Although anti-IL-4Rα antibodies containing human sequences (e.g., human heavy and light chain variable domain sequences comprising human CDR sequences) are extensively discussed herein, non-human anti-IL-4Rα antibodies are also contemplated. In some embodiments, non-human anti-IL-4Rα antibodies comprise human CDR sequences from an anti-IL-4Rα antibody as described herein and non-human framework sequences. Non-human framework sequences include, in some embodiments, any sequence that can be used for generating synthetic heavy and/or light chain variable domains using one or more human CDR sequences as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. In some embodiments, a non-human anti-IL-4Rα antibody includes an anti-IL-4Rα antibody generated by grafting one or more human CDR sequences as described herein onto a non-human framework sequence (e.g., a mouse or chicken framework sequence).

The complete amino acid sequence of an exemplary human IL-4Rα comprises or consists of the amino acid sequence of SEQ ID NO: 83. The amino acid sequence of the extracellular domain of an exemplary human IL-4Rα comprises or consists of the amino acid sequence of SEQ ID NO: 82.

In some embodiments, the anti-IL-4Rα antibody described herein specifically recognizes an epitope within human IL-4Rα. In some embodiments, the anti-IL-4Rα antibody cross-reacts with IL-4Rα from species other than human species. In some embodiments, the anti-IL-4Rα antibody is completely specific for human IL-4Rα and does not exhibit cross-reactivity with IL-4Rα from other non-human species.

In some embodiments, the anti-IL-4Rα antibody cross-reacts with at least one allelic variant of the IL-4Rα protein (or fragments thereof). In some embodiments, the allelic variant has up to about 30 (such as about any of 1, 2, 3, 4, 5, 6, 7, 8, 9, 10, 15, 20, 25, or 30) amino acid substitutions (such as a conservative substitution) when compared to the naturally occurring IL-4Rα (or fragments thereof). In some embodiments, the anti-IL-4Rα antibody does not cross-react with any allelic variant of the IL-4Rα protein (or fragments thereof).

In some embodiments, the anti-IL-4Rα antibody cross-reacts with at least one interspecies variant of the IL-4Rα protein. In some embodiments, for example, the IL-4Rα protein (or fragments thereof) is human IL-4Rα and the interspecies variant of the IL-4Rα protein (or fragments thereof) is a cynomolgus monkey variant or marmoset monkey variant thereof. In some embodiments, the anti-IL-4Rα antibody does not cross-react with any interspecies variant of the IL-4Rα protein.

In some embodiments, according to any of the anti-IL-4Rα antibodies described herein, the anti-IL-4Rα antibody comprises an antibody heavy chain constant region and an antibody light chain constant region. In some embodiments, the anti-IL-4Rα antibody comprises an IgG1 heavy chain constant region. In some embodiments, the anti-IL-4Rα antibody comprises an IgG2 heavy chain constant region. In some embodiments, the anti-IL-4Rα antibody comprises an IgG3 heavy chain constant region. In some embodiments, the anti-IL-4Rα antibody comprises an IgG4 heavy chain constant region. In some embodiments, the heavy chain constant region comprises (including consisting of or consisting essentially of) the amino acid sequence of SEQ ID NO: 84. In some embodiments, the heavy chain constant region comprises (including consisting of or consisting essentially of) the amino acid sequence of SEQ ID NO: 85. In some embodiments, the anti-IL-4Rα comprises a lambda light chain constant region. In some embodiments, the anti-IL-4Rα antibody comprises a kappa light chain constant region. In some embodiments, the light chain constant region comprises (including consisting of or consisting essentially of) the amino acid sequence of SEQ ID NO: 86. In some embodiments, the anti-IL-4Rα antibody comprises an antibody heavy chain variable domain and an antibody light chain variable domain.

In some embodiments, the anti-IL-4Rα antibody comprises a V_{H} comprising an HC-CDR1 comprising SYAMH (SEQ ID NO: 1); an HC-CDR2 comprising GISX₁X₂X₃X₄STYYANSVKG (SEQ ID NO: 78), wherein X₁ is P, S, H, G, or Y, X₂ is S, T, or N, X₃ is G or S, X₄ is 5, V, G, T, A, or N; and an HC-CDR3 comprising X₁X₂X₃X₄YRGGMDV (SEQ ID NO: 79), wherein X₁ is V or S, X₂ is K, F, or R, X₃ is P, V, G, R, S, or L, X₄ is G, A, R, K, or L; and a V_{L} comprising an LC-CDR1 comprising RASQX₁X₂SX₃AYLA (SEQ ID NO: 80), wherein X₁ is G, S, N, or D, X₂ is I, V, or A, X₃ is S, T, or N; an LC-CDR2 comprising GTSRRAT (SEQ ID NO: 41); and an LC-CDR3 comprising QLYGX₁X₂SVT (SEQ ID NO: 81), wherein X₁ is A, S, T, or R, X₂ is T or S.

In some embodiments, the anti-IL-4Rα antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30.

In some embodiments, the anti-IL-4Rα antibody comprises a V_{L} comprising: an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions.

In some embodiments, the anti-IL-4Rα antibody comprises a V_{L} comprising: an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47.

In some embodiments, the anti-IL-4Rα antibody comprises a V_{H} comprising: an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions; and a V_{L} comprising: an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30; and a V_{L} comprising: an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17, or a variant thereof comprising up to about 5 amino acid substitutions in the HC-CDRs; and a V_{L} comprising: an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24, or a variant thereof comprising up to about 5 amino acid substitutions in the HC-CDRs; and a V_{L} comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24; and a V_{L} comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25, or a variant thereof comprising up to about 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26, or a variant thereof comprising up to about 5 amino acid substitutions in the HC-CDRs; and a V_{L} comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26; and a V_{L} comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27, or a variant thereof comprising up to about 5 amino acid substitutions in the HC-CDRs; and a V_{L} comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27; and a V_{L} comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29, or a variant thereof comprising up to about 5 amino acid substitutions in the HC-CDRs; and a V_{L} comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29; and a V_{L} comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30, or a variant thereof comprising up to about 5 amino acid substitutions in the HC-CDRs; and a V_{L} comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30; and a V_{L} comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47, or a variant thereof comprising up to about 5 amino acid substitutions in the LC-CDRs.

In some embodiments, the anti-IL-4Rα antibody comprises a V_{H} comprising: an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47.

In some embodiments, the anti-IL-4Rα antibody comprises a V_{H} comprising the amino acid sequence of any one of SEQ ID NOs: 48-64, or a variant thereof comprising up to about 5 amino acid substitutions; and a V_{L} comprising the amino acid sequence of any one of SEQ ID NOs: 65-77, or a variant thereof comprising up to about 5 amino acid substitutions. In some embodiments, the anti-IL-4Rα antibody comprises a V_{H} comprising the amino acid sequence of any one of SEQ ID NOs: 48-64; and a V_{L} comprising the amino acid sequence of any one of SEQ ID NOs: 65-77.

In some embodiments, the anti-IL-4Rα antibody comprises a V_{H} comprising the amino acid sequences of SEQ ID NOs: 1, 2 and 17, or a variant thereof comprising up to about 5 amino acid substitutions; and a V_{L} comprising the amino acid sequences of SEQ ID NOs: 31, 41 and 42, or a variant thereof comprising up to about 5 amino acid substitutions. In some embodiments, the anti-IL-4Rα antibody comprises a V_{H} comprising the amino acid sequences of SEQ ID NOs: 1, 2 and 17; and a V_{L} comprising the amino acid sequences of SEQ ID NOs: 31, 41 and 42.

In some embodiments, the anti-IL-4Rα antibody comprises a V_{H} comprising the amino acid sequences of SEQ ID NOs: 1, 3 and 18, or a variant thereof comprising up to about 5 amino acid substitutions; and a V_{L} comprising the amino acid sequences of SEQ ID NOs: 32, 41 and 43, or a variant thereof comprising up to about 5 amino acid substitutions. In some embodiments, the anti-IL-4Rα antibody comprises a V_{H} comprising the amino acid sequences of SEQ ID NOs: 1, 3 and 18; and a V_{L} comprising the amino acid sequences of SEQ ID NOs: 32, 41 and 43.

In some embodiments, the anti-IL-4Rα antibody comprises a V_{H} comprising the amino acid sequences of SEQ ID NOs: 1, 4 and 19, or a variant thereof comprising up to about 5 amino acid substitutions; and a V_{L} comprising the amino acid sequences of SEQ ID NOs: 32, 41 and 43, or a variant thereof comprising up to about 5 amino acid substitutions. In some embodiments, the anti-IL-4Rα antibody comprises a V_{H} comprising the amino acid sequences of SEQ ID NOs: 1, 4 and 19; and a V_{L} comprising the amino acid sequences of SEQ ID NOs: 32, 41 and 43.

In some embodiments, the anti-IL-4Rα antibody comprises a V_{H} comprising the amino acid sequences of SEQ ID NOs: 1, 5 and 20, or a variant thereof comprising up to about 5 amino acid substitutions; and a V_{L} comprising the amino acid sequences of SEQ ID NOs: 31, 41 and 44, or a variant thereof comprising up to about 5 amino acid substitutions. In some embodiments, the anti-IL-4Rα antibody comprises a V_{H} comprising the amino acid sequences of SEQ ID NOs: 1, 5 and 20; and a V_{L} comprising the amino acid sequences of SEQ ID NOs: 31, 41 and 44.

In some embodiments, the anti-IL-4Rα antibody comprises a V_{H} comprising the amino acid sequences of SEQ ID NOs: 1, 6 and 21, or a variant thereof comprising up to about 5 amino acid substitutions; and a V_{L} comprising the amino acid sequences of SEQ ID NOs: 31, 41 and 44, or a variant thereof comprising up to about 5 amino acid substitutions. In some embodiments, the anti-IL-4Rα antibody comprises a V_{H} comprising the amino acid sequences of SEQ ID NOs: 1, 6 and 21; and a V_{L} comprising the amino acid sequences of SEQ ID NOs: 31, 41 and 44.

In some embodiments, the anti-IL-4Rα antibody comprises a V_{H} comprising the amino acid sequences of SEQ ID NOs: 1, 7 and 22, or a variant thereof comprising up to about 5 amino acid substitutions; and a V_{L} comprising the amino acid sequences of SEQ ID NOs: 32, 41 and 43, or a variant thereof comprising up to about 5 amino acid substitutions. In some embodiments, the anti-IL-4Rα antibody comprises a V_{H} comprising the amino acid sequences of SEQ ID NOs: 1, 7 and 22; and a V_{L} comprising the amino acid sequences of SEQ ID NOs: 32, 41 and 43.

In some embodiments, the anti-IL-4Rα antibody comprises a V_{H} comprising the amino acid sequences of SEQ ID NOs: 1, 8 and 23, or a variant thereof comprising up to about 5 amino acid substitutions; and a V_{L} comprising the amino acid sequences of SEQ ID NOs: 31, 41 and 44, or a variant thereof comprising up to about 5 amino acid substitutions. In some embodiments, the anti-IL-4Rα antibody comprises a V_{H} comprising the amino acid sequences of SEQ ID NOs: 1, 8 and 23; and a V_{L} comprising the amino acid sequences of SEQ ID NOs: 31, 41 and 44.

In some embodiments, the anti-IL-4Rα antibody comprises a V_{H} comprising the amino acid sequences of SEQ ID NOs: 1, 9 and 24, or a variant thereof comprising up to about 5 amino acid substitutions; and a V_{L} comprising the amino acid sequences of SEQ ID NOs: 33, 41 and 44, or a variant thereof comprising up to about 5 amino acid substitutions. In some embodiments, the anti-IL-4Rα antibody comprises a V_{H} comprising the amino acid sequences of SEQ ID NOs: 1, 9 and 24; and a V_{L} comprising the amino acid sequences of SEQ ID NOs: 33, 41 and 44.

In some embodiments, the anti-IL-4Rα antibody comprises a V_{H} comprising the amino acid sequences of SEQ ID NOs: 1, 10 and 25, or a variant thereof comprising up to about 5 amino acid substitutions; and a V_{L} comprising the amino acid sequences of SEQ ID NOs: 34, 41 and 43, or a variant thereof comprising up to about 5 amino acid substitutions. In some embodiments, the anti-IL-4Rα antibody comprises a V_{H} comprising the amino acid sequences of SEQ ID NOs: 1, 10 and 25; and a V_{L} comprising the amino acid sequences of SEQ ID NOs: 34, 41 and 43.

In some embodiments, the anti-IL-4Rα antibody comprises a V_{H} comprising the amino acid sequences of SEQ ID NOs: 1, 11 and 26, or a variant thereof comprising up to about 5 amino acid substitutions; and a V_{L} comprising the amino acid sequences of SEQ ID NOs: 35, 41 and 43, or a variant thereof comprising up to about 5 amino acid substitutions. In some embodiments, the anti-IL-4Rα antibody comprises a V_{H} comprising the amino acid sequences of SEQ ID NOs: 1, 11 and 26; and a V_{L} comprising the amino acid sequences of SEQ ID NOs: 35, 41 and 43.

In some embodiments, the anti-IL-4Rα antibody comprises a V_{H} comprising the amino acid sequences of SEQ ID NOs: 1, 4 and 19, or a variant thereof comprising up to about 5 amino acid substitutions; and a V_{L} comprising the amino acid sequences of SEQ ID NOs: 36, 41 and 43, or a variant thereof comprising up to about 5 amino acid substitutions. In some embodiments, the anti-IL-4Rα antibody comprises a V_{H} comprising the amino acid sequences of SEQ ID NOs: 1, 4 and 19; and a V_{L} comprising the amino acid sequences of SEQ ID NOs: 36, 41 and 43.

In some embodiments, the anti-IL-4Rα antibody comprises a V_{H} comprising the amino acid sequences of SEQ ID NOs: 1, 2 and 27, or a variant thereof comprising up to about 5 amino acid substitutions; and a V_{L} comprising the amino acid sequences of SEQ ID NOs: 37, 41 and 42, or a variant thereof comprising up to about 5 amino acid substitutions. In some embodiments, the anti-IL-4Rα antibody comprises a V_{H} comprising the amino acid sequences of SEQ ID NOs: 1, 2 and 27; and a V_{L} comprising the amino acid sequences of SEQ ID NOs: 37, 41 and 42.

In some embodiments, the anti-IL-4Rα antibody comprises a V_{H} comprising the amino acid sequences of SEQ ID NOs: 1, 12 and 28, or a variant thereof comprising up to about 5 amino acid substitutions; and a V_{L} comprising the amino acid sequences of SEQ ID NOs: 38, 41 and 45, or a variant thereof comprising up to about 5 amino acid substitutions. In some embodiments, the anti-IL-4Rα antibody comprises a V_{H} comprising the amino acid sequences of SEQ ID NOs: 1, 12 and 28; and a V_{L} comprising the amino acid sequences of SEQ ID NOs: 38, 41 and 45.

In some embodiments, the anti-IL-4Rα antibody comprises a V_{H} comprising the amino acid sequences of SEQ ID NOs: 1, 13 and 28, or a variant thereof comprising up to about 5 amino acid substitutions; and a V_{L} comprising the amino acid sequences of SEQ ID NOs: 36, 41 and 46, or a variant thereof comprising up to about 5 amino acid substitutions. In some embodiments, the anti-IL-4Rα antibody comprises a V_{H} comprising the amino acid sequences of SEQ ID NOs: 1, 13 and 28; and a V_{L} comprising the amino acid sequences of SEQ ID NOs: 36, 41 and 46.

In some embodiments, the anti-IL-4Rα antibody comprises a V_{H} comprising the amino acid sequences of SEQ ID NOs: 1, 14 and 29, or a variant thereof comprising up to about 5 amino acid substitutions; and a V_{L} comprising the amino acid sequences of SEQ ID NOs: 39, 41 and 43, or a variant thereof comprising up to about 5 amino acid substitutions. In some embodiments, the anti-IL-4Rα antibody comprises a V_{H} comprising the amino acid sequences of SEQ ID NOs: 1, 14 and 29; and a V_{L} comprising the amino acid sequences of SEQ ID NOs: 39, 41 and 43.

In some embodiments, the anti-IL-4Rα antibody comprises a V_{H} comprising the amino acid sequences of SEQ ID NOs: 1, 15 and 20, or a variant thereof comprising up to about 5 amino acid substitutions; and a V_{L} comprising the amino acid sequences of SEQ ID NOs: 34, 41 and 43, or a variant thereof comprising up to about 5 amino acid substitutions. In some embodiments, the anti-IL-4Rα antibody comprises a V_{H} comprising the amino acid sequences of SEQ ID NOs: 1, 15 and 20; and a V_{L} comprising the amino acid sequences of SEQ ID NOs: 34, 41 and 43.

In some embodiments, the anti-IL-4Rα antibody comprises a V_{H} comprising the amino acid sequences of SEQ ID NOs: 1, 7 and 30, or a variant thereof comprising up to about 5 amino acid substitutions; and a V_{L} comprising the amino acid sequences of SEQ ID NOs: 40, 41 and 43, or a variant thereof comprising up to about 5 amino acid substitutions. In some embodiments, the anti-IL-4Rα antibody comprises a V_{H} comprising the amino acid sequences of SEQ ID NOs: 1, 7 and 30; and a V_{L} comprising the amino acid sequences of SEQ ID NOs: 40, 41 and 43.

In some embodiments, the anti-IL-4Rα antibody comprises a V_{H} comprising the amino acid sequences of SEQ ID NOs: 1, 16 and 20, or a variant thereof comprising up to about 5 amino acid substitutions; and a V_{L} comprising the amino acid sequences of SEQ ID NOs: 38, 41 and 47, or a variant thereof comprising up to about 5 amino acid substitutions. In some embodiments, the anti-IL-4Rα antibody comprises a V_{H} comprising the amino acid sequences of SEQ ID NOs: 1, 16 and 20; and a V_{L} comprising the amino acid sequences of SEQ ID NOs: 38, 41 and 47.

In some embodiments, the amino acid substitutions described above are limited to “exemplary substitutions” shown in Table 6 of this application. In some embodiments, the amino acid substitutions are limited to “preferred substitutions” shown in Table 6 of this application.

In some embodiments, the anti-IL-4Rα antibody comprises a V_{H} comprising an HC-CDR1, an HC-CDR2 and an HC-CDR3 of the V_{H} comprising the amino acid sequence of any one of SEQ ID NOs: 48-64; and a V_{L} comprising an LC-CDR1, an LC-CDR2, and an LC-CDR3 of the V_{L} comprising the amino acid sequence of any one of SEQ ID NOs: 65-77.

In some embodiments, the anti-IL-4Rα antibody comprises a V_{H} comprising one, two or three HC-CDRs of SEQ ID NO: 48. In some embodiments, the anti-IL-4Rα antibody comprises a V_{H} comprising one, two or three HC-CDRs of SEQ ID NO: 49. In some embodiments, the anti-IL-4Rα antibody comprises a V_{H} comprising one, two or three HC-CDRs of SEQ ID NO: 50. In some embodiments, the anti-IL-4Rα antibody comprises a Vx comprising one, two or three HC-CDRs of SEQ ID NO: 51. In some embodiments, the anti-IL-4Rα antibody comprises a V_{H} comprising one, two or three HC-CDRs of SEQ ID NO: 52. In some embodiments, the anti-IL-4Rα antibody comprises a V_{H} comprising one, two or three HC-CDRs of SEQ ID NO: 53. In some embodiments, the anti-IL-4Rα antibody comprises a V_{H} comprising one, two or three HC-CDRs of SEQ ID NO: 54. In some embodiments, the anti-IL-4Rα antibody comprises a V_{H} comprising one, two or three HC-CDRs of SEQ ID NO: 55. In some embodiments, the anti-IL-4Rα antibody comprises a V_{H} comprising one, two or three HC-CDRs of SEQ ID NO: 56. In some embodiments, the anti-IL-4Rα antibody comprises a V_{H} comprising one, two or three HC-CDRs of SEQ ID NO: 57. In some embodiments, the anti-IL-4Rα antibody comprises a V_{H} comprising one, two or three HC-CDRs of SEQ ID NO: 58. In some embodiments, the anti-IL-4Rα antibody comprises a V_{H} comprising one, two or three HC-CDRs of SEQ ID NO: 59. In some embodiments, the anti-IL-4Rα antibody comprises a V_{H} comprising one, two or three HC-CDRs of SEQ ID NO: 60. In some embodiments, the anti-IL-4Rα antibody comprises a Vx comprising one, two or three HC-CDRs of SEQ ID NO: 61. In some embodiments, the anti-IL-4Rα antibody comprises a V_{H} comprising one, two or three HC-CDRs of SEQ ID NO: 62. In some embodiments, the anti-IL-4Rα antibody comprises a V_{H} comprising one, two or three HC-CDRs of SEQ ID NO: 63. In some embodiments, the anti-IL-4Rα antibody comprises a V_{H} comprising one, two or three HC-CDRs of SEQ ID NO: 64.

In some embodiments, the anti-IL-4Rα antibody comprises a V_{L} comprising one, two or three LC-CDRs of SEQ ID NO: 65. In some embodiments, the anti-IL-4Rα antibody comprises a V_{L} comprising one, two or three LC-CDRs of SEQ ID NO: 66. In some embodiments, the anti-IL-4Rα antibody comprises a V_{L} comprising one, two or three LC-CDRs of SEQ ID NO: 67. In some embodiments, the anti-IL-4Rα antibody comprises a V_{L} comprising one, two or three LC-CDRs of SEQ ID NO: 68. In some embodiments, the anti-IL-4Rα antibody comprises a V_{L} comprising one, two or three LC-CDRs of SEQ ID NO: 69. In some embodiments, the anti-IL-4Rα antibody comprises a V_{L} comprising one, two or three LC-CDRs of SEQ ID NO: 70. In some embodiments, the anti-IL-4Rα antibody comprises a V_{L} comprising one, two or three LC-CDRs of SEQ ID NO: 71. In some embodiments, the anti-IL-4Rα antibody comprises a V_{L} comprising one, two or three LC-CDRs of SEQ ID NO: 72. In some embodiments, the anti-IL-4Rα antibody comprises a V_{L} comprising one, two or three LC-CDRs of SEQ ID NO: 73. In some embodiments, the anti-IL-4Rα antibody comprises a V_{L} comprising one, two or three LC-CDRs of SEQ ID NO: 74. In some embodiments, the anti-IL-4Rα antibody comprises a V_{L} comprising one, two or three LC-CDRs of SEQ ID NO: 75. In some embodiments, the anti-IL-4Rα antibody comprises a V_{L} comprising one, two or three LC-CDRs of SEQ ID NO: 76. In some embodiments, the anti-IL-4Rα antibody comprises a V_{L} comprising one, two or three LC-CDRs of SEQ ID NO: 77.

In some embodiments, the anti-IL-4Rα antibody comprises a V_{H} comprising HC-CDR1, HC-CDR2 and HC-CDR3 of the V_{H} of SEQ ID NO: 48, and a V_{L} comprising LC-CDR1, LC-CDR2 and LC-CDR3 of the V_{L} of SEQ ID NO: 65. In some embodiments, the anti-IL-4Rα antibody comprises a V_{H} comprising HC-CDR1, HC-CDR2 and HC-CDR3 of the V_{H} of SEQ ID NO: 49, and a V_{L} comprising LC-CDR1, LC-CDR2 and LC-CDR3 of the V_{L} of SEQ ID NO: 66. In some embodiments, the anti-IL-4Rα antibody comprises a V_{H} comprising HC-CDR1, HC-CDR2 and HC-CDR3 of the V_{H} of SEQ ID NO: 50, and a V_{L} comprising LC-CDR1, LC-CDR2 and LC-CDR3 of the V_{L} of SEQ ID NO: 66. In some embodiments, the anti-IL-4Rα antibody comprises a V_{H} comprising HC-CDR1, HC-CDR2 and HC-CDR3 of the V_{H} of SEQ ID NO: 51, and a V_{L} comprising LC-CDR1, LC-CDR2 and LC-CDR3 of the V_{L} of SEQ ID NO: 67. In some embodiments, the anti-IL-4Rα antibody comprises a V_{H} comprising HC-CDR1, HC-CDR2 and HC-CDR3 of the V_{H} of SEQ ID NO: 52, and a V_{L} comprising LC-CDR1, LC-CDR2 and LC-CDR3 of the V_{L} of SEQ ID NO: 67. In some embodiments, the anti-IL-4Rα antibody comprises a V_{H} comprising HC-CDR1, HC-CDR2 and HC-CDR3 of the V_{H} of SEQ ID NO: 53, and a V_{L} comprising LC-CDR1, LC-CDR2 and LC-CDR3 of the V_{L} of SEQ ID NO: 66. In some embodiments, the anti-IL-4Rα antibody comprises a V_{H} comprising HC-CDR1, HC-CDR2 and HC-CDR3 of the V_{H} of SEQ ID NO: 54, and a V_{L} comprising LC-CDR1, LC-CDR2 and LC-CDR3 of the V_{L} of SEQ ID NO: 67. In some embodiments, the anti-IL-4Rα antibody comprises a V_{H} comprising HC-CDR1, HC-CDR2 and HC-CDR3 of the V_{H} of SEQ ID NO: 55, and a V_{L} comprising LC-CDR1, LC-CDR2 and LC-CDR3 of the V_{L} of SEQ ID NO: 68. In some embodiments, the anti-IL-4Rα antibody comprises a V_{H} comprising HC-CDR1, HC-CDR2 and HC-CDR3 of the V_{H} of SEQ ID NO: 56, and a V_{L} comprising LC-CDR1, LC-CDR2 and LC-CDR3 of the V_{L} of SEQ ID NO: 69. In some embodiments, the anti-IL-4Rα antibody comprises a V_{H} comprising HC-CDR1, HC-CDR2 and HC-CDR3 of the V_{H} of SEQ ID NO: 57, and a V_{L} comprising LC-CDR1, LC-CDR2 and LC-CDR3 of the V_{L} of SEQ ID NO: 70. In some embodiments, the anti-IL-4Rα antibody comprises a V_{H} comprising HC-CDR1, HC-CDR2 and HC-CDR3 of the V_{H} of SEQ ID NO: 50, and a V_{L} comprising LC-CDR1, LC-CDR2 and LC-CDR3 of the V_{L} of SEQ ID NO: 71. In some embodiments, the anti-IL-4Rα antibody comprises a V_{H} comprising HC-CDR1, HC-CDR2 and HC-CDR3 of the V_{H} of SEQ ID NO: 58, and a V_{L} comprising LC-CDR1, LC-CDR2 and LC-CDR3 of the V_{L} of SEQ ID NO: 72. In some embodiments, the anti-IL-4Rα antibody comprises a V_{H} comprising HC-CDR1, HC-CDR2 and HC-CDR3 of the V_{H} of SEQ ID NO: 59, and a V_{L} comprising LC-CDR1, LC-CDR2 and LC-CDR3 of the V_{L} of SEQ ID NO: 73. In some embodiments, the anti-IL-4Rα antibody comprises a V_{H} comprising HC-CDR1, HC-CDR2 and HC-CDR3 of the V_{H} of SEQ ID NO: 60, and a V_{L} comprising LC-CDR1, LC-CDR2 and LC-CDR3 of the V_{L} of SEQ ID NO: 74. In some embodiments, the anti-IL-4Rα antibody comprises a V_{H} comprising HC-CDR1, HC-CDR2 and HC-CDR3 of the V_{H} of SEQ ID NO: 61, and a V_{L} comprising LC-CDR1, LC-CDR2 and LC-CDR3 of the V_{L} of SEQ ID NO: 75. In some embodiments, the anti-IL-4Rα antibody comprises a V_{H} comprising HC-CDR1, HC-CDR2 and HC-CDR3 of the V_{H} of SEQ ID NO: 62, and a V_{L} comprising LC-CDR1, LC-CDR2 and LC-CDR3 of the V_{L} of SEQ ID NO: 69. In some embodiments, the anti-IL-4Rα antibody comprises a V_{H} comprising HC-CDR1, HC-CDR2 and HC-CDR3 of the V_{H} of SEQ ID NO: 63, and a V_{L} comprising LC-CDR1, LC-CDR2 and LC-CDR3 of the V_{L} of SEQ ID NO: 76. In some embodiments, the anti-IL-4Rα antibody comprises a V_{H} comprising HC-CDR1, HC-CDR2 and HC-CDR3 of the V_{H} of SEQ ID NO: 64, and a V_{L} comprising LC-CDR1, LC-CDR2 and LC-CDR3 of the V_{L} of SEQ ID NO: 77.

In some embodiments, the anti-IL-4Rα antibody comprises a V_{H} comprising the amino acid sequence of any one of SEQ ID NOs: 48-64, or a variant thereof having at least about 90% (for example at least about any of 91%, 92%, 93%, 94%, 95%, 96%, 97%, 98%, or 99%) sequence identity, and a V_{L} comprising the amino acid sequence of any one of SEQ ID NOs: 65-77, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of any one of SEQ ID NOs: 48-64, and a V_{L} comprising the amino acid sequence of any one of SEQ ID NOs: 65-77.

In some embodiments, the anti-IL-4Rα antibody comprises a V_{H} comprising the amino acid sequence of SEQ ID NO: 48, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 65, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 48 and a V_{L} comprising the amino acid sequence of SEQ ID NO: 65.

In some embodiments, the anti-IL-4Rα antibody comprises a V_{H} comprising the amino acid sequence of SEQ ID NO: 49, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 66, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 49 and a V_{L} comprising the amino acid sequence of SEQ ID NO: 66.

In some embodiments, the anti-IL-4Rα antibody comprises a V_{H} comprising the amino acid sequence of SEQ ID NO: 50, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 66, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 50 and a V_{L} comprising the amino acid sequence of SEQ ID NO: 66.

In some embodiments, the anti-IL-4Rα antibody comprises a V_{H} comprising the amino acid sequence of SEQ ID NO: 51, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 67, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 51 and a V_{L} comprising the amino acid sequence of SEQ ID NO: 67.

In some embodiments, the anti-IL-4Rα antibody comprises a V_{H} comprising the amino acid sequence of SEQ ID NO: 52, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 67, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 52 and a V_{L} comprising the amino acid sequence of SEQ ID NO: 67.

In some embodiments, the anti-IL-4Rα antibody comprises a V_{H} comprising the amino acid sequence of SEQ ID NO: 53, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 66, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 53 and a V_{L} comprising the amino acid sequence of SEQ ID NO: 66.

In some embodiments, the anti-IL-4Rα antibody comprises a V_{H} comprising the amino acid sequence of SEQ ID NO: 54, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 67, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 54 and a V_{L} comprising the amino acid sequence of SEQ ID NO: 67.

In some embodiments, the anti-IL-4Rα antibody comprises a V_{H} comprising the amino acid sequence of SEQ ID NO: 55, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 68, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 55 and a V_{L} comprising the amino acid sequence of SEQ ID NO: 68.

In some embodiments, the anti-IL-4Rα antibody comprises a V_{H} comprising the amino acid sequence of SEQ ID NO: 56, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 69, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 56 and a V_{L} comprising the amino acid sequence of SEQ ID NO: 69.

In some embodiments, the anti-IL-4Rα antibody comprises a V_{H} comprising the amino acid sequence of SEQ ID NO: 57, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 70, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 57 and a V_{L} comprising the amino acid sequence of SEQ ID NO: 70.

In some embodiments, the anti-IL-4Rα antibody comprises a V_{H} comprising the amino acid sequence of SEQ ID NO: 50, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 71, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 50 and a V_{L} comprising the amino acid sequence of SEQ ID NO: 71.

In some embodiments, the anti-IL-4Rα antibody comprises a V_{H} comprising the amino acid sequence of SEQ ID NO: 58, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 72, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 58 and a V_{L} comprising the amino acid sequence of SEQ ID NO: 72.

In some embodiments, the anti-IL-4Rα antibody comprises a V_{H} comprising the amino acid sequence of SEQ ID NO: 59, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 73, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 59 and a V_{L} comprising the amino acid sequence of SEQ ID NO: 73.

In some embodiments, the anti-IL-4Rα antibody comprises a V_{H} comprising the amino acid sequence of SEQ ID NO: 60, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 74, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 60 and a V_{L} comprising the amino acid sequence of SEQ ID NO: 74.

In some embodiments, the anti-IL-4Rα antibody comprises a V_{H} comprising the amino acid sequence of SEQ ID NO: 61, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 75, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 61 and a V_{L} comprising the amino acid sequence of SEQ ID NO: 75.

In some embodiments, the anti-IL-4Rα antibody comprises a V_{H} comprising the amino acid sequence of SEQ ID NO: 62, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 69, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 62 and a V_{L} comprising the amino acid sequence of SEQ ID NO: 69.

In some embodiments, the anti-IL-4Rα antibody comprises a V_{H} comprising the amino acid sequence of SEQ ID NO: 63, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 76, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 63 and a V_{L} comprising the amino acid sequence of SEQ ID NO: 76.

In some embodiments, the anti-IL-4Rα antibody comprises a V_{H} comprising the amino acid sequence of SEQ ID NO: 64, or a variant thereof having at least about 90% (for example at least about any of 91%, 92%, 93%, 94%, 95%, 96%, 97%, 98%, or 99%) sequence identity, and a V_{L} comprising the amino acid sequence of SEQ ID NO: 77, or a variant thereof having at least about 90% (for example at least about any of 91%, 92%, 93%, 94%, 95%, 96%, 97%, 98%, or 99%) sequence identity. In some embodiments, the anti-IL-4Rα antibody comprises a V_{H} comprising the amino acid sequence of SEQ ID NO: 64 and a V_{L} comprising the amino acid sequence of SEQ ID NO: 77.

In some embodiments, functional epitopes can be mapped by combinatorial alanine scanning. In this process, a combinatorial alanine-scanning strategy can be used to identify amino acids in the IL-4Rα protein that are necessary for interaction with IL-4Rα antibodies. In some embodiments, the epitope is conformational and the crystal structure of anti-IL-4Rα antibodies bound to IL-4Rα may be employed to identify the epitopes.

In some embodiments, the present application provides antibodies which compete with any one of the IL-4Rα antibodies described herein for binding to IL-4Rα. In some embodiments, the present application provides antibodies which compete with any one of the anti-IL-4Rα antibodies provided herein for binding to an epitope on the IL-4Rα. In some embodiments, an anti-IL-4Rα antibody is provided that binds to the same epitope as an anti-IL-4Rα antibody comprising a V_{H} comprising the amino acid sequence of any one of SEQ ID NOs: 48-64, and a V_{L} comprising the amino acid sequence of any one of SEQ ID NOs: 65-77. In some embodiments, an anti-IL-4Rα antibody is provided that specifically binds to IL-4Rα competitively with an anti-IL-4Rα antibody comprising a V_{H} comprising the amino acid sequence of any one of SEQ ID NOs: 48-64, and a V_{L} comprising the amino acid sequence of any one of SEQ ID NOs: 65-77.

In some embodiments, competition assays may be used to identify a monoclonal antibody that competes with an anti-IL-4Rα antibody described herein for binding to IL-4Rα. Competition assays can be used to determine whether two antibodies bind to the same epitope by recognizing identical or sterically overlapping epitopes or one antibody competitively inhibits binding of another antibody to the antigen. In certain embodiments, such a competing antibody binds to the same epitope that is bound by an antibody described herein. Exemplary competition assays include, but are not limited to, routine assays such as those provided in Harlow and Lane (1988) Antibodies: A Laboratory Manual ch. 14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). In some embodiments, two antibodies are said to bind to the same epitope if each blocks binding of the other by 50% or more. In some embodiments, the antibody that competes with an anti-IL-4Rα antibody described herein is a chimeric, humanized or human antibody.

Exemplary anti-IL-4Rα antibody sequences are shown in Tables 2, 3, and 4, wherein the CDR numbering is according to the EU index of Kabat. Those skilled in the art will recognize that many algorithms are known for prediction of CDR positions and for delimitation of antibody heavy chain and light chain variable regions. Anti-IL-4Rα antibodies comprising CDRs, V_{H} and/or V_{L} sequences from antibodies described herein, but based on prediction algorithms other than those exemplified in the tables below, are within the scope of this invention.

TABLE 2
Exemplary anti-IL-4Rα antibody CDR sequences.

| Antibody | | | |
|---|---|---|---|
| Name | HC-CDR1 | HC-CDR2 | HC-CDR3 |
| C27 | SYAMH | GISSNGGSTYYANSVKG | VKVGYRGGMDV |
| | (SEQ ID NO: 1) | (SEQ ID NO: 2) | (SEQ ID NO: 17) |
| C27-6-33 | SYAMH | GISPSGSSTYYANSVKG | SKVRYRGGMDV |
| | (SEQ ID NO: 1) | (SEQ ID NO: 3) | (SEQ ID NO: 18) |
| C27-7-33 | SYAMH | GISPSGVSTYYANSVKG | VKVKYRGGMDV |
| | (SEQ ID NO: 1) | (SEQ ID NO: 4) | (SEQ ID NO: 19) |
| C27-24-56 | SYAMH | GISPTSGSTYYANSVKG | VKVRYRGGMDV |
| | (SEQ ID NO: 1) | (SEQ ID NO: 5) | (SEQ ID NO: 20) |
| C27-47-56 | SYAMH | GISPTGTSTYYANSVKG | VKGAYRGGMDV |
| | (SEQ ID NO: 1) | (SEQ ID NO: 6) | (SEQ ID NO: 21) |
| C27-33-33 | SYAMH | GISSSGSSTYYANSVKG | VKVAYRGGMDV |
| | (SEQ ID NO: 1) | (SEQ ID NO: 7) | (SEQ ID NO: 22) |
| C27-56-56 | SYAMH | GISPSSTSTYYANSVKG | VKVLYRGGMDV |
| | (SEQ ID NO: 1) | (SEQ ID NO: 8) | (SEQ ID NO: 23) |
| C27-78-78 | SYAMH | GISPSSASTYYANSVKG | VKSKYRGGMDV |
| | (SEQ ID NO: 1) | (SEQ ID NO: 9) | (SEQ ID NO: 24) |
| C27-82-58 | SYAMH | GISGNSASTYYANSVKG | VKLKYRGGMDV |
| | (SEQ ID NO: 1) | (SEQ ID NO: 10) | (SEQ ID NO: 25) |
| C27-54-54 | SYAMH | GISHSGTSTYYANSVKG | VRVLYRGGMDV |
| | (SEQ ID NO: 1) | (SEQ ID NO: 11) | (SEQ ID NO: 26) |
| C27-36-36 | SYAMH | GISPSGVSTYYANSVKG | VKVKYRGGMDV |
| | (SEQ ID NO: 1) | (SEQ ID NO: 4) | (SEQ ID NO: 19) |
| C27-53-53 | SYAMH | GISSNGGSTYYANSVKG | VFVRYRGGMDV |
| | (SEQ ID NO: 1) | (SEQ ID NO: 2) | (SEQ ID NO: 27) |
| C27-67-67 | SYAMH | GISPTSASTYYANSVKG | VKGRYRGGMDV |
| | (SEQ ID NO: 1) | (SEQ ID NO: 12) | (SEQ ID NO: 28) |
| C27-55-55 | SYAMH | GISPTGGSTYYANSVKG | VKGRYRGGMDV |
| | (SEQ ID NO: 1) | (SEQ ID NO: 13) | (SEQ ID NO: 28) |
| C27-59-59 | SYAMH | GISHSGNSTYYANSVKG | VKRRYRGGMDV |
| | (SEQ ID NO: 1) | (SEQ ID NO: 14) | (SEQ ID NO: 29) |
| C27-58-58 | SYAMH | GISPSSNSTYYANSVKG | VKVRYRGGMDV |
| | (SEQ ID NO: 1) | (SEQ ID NO: 15) | (SEQ ID NO: 20) |
| C27-52-52 | SYAMH | GISSSGSSTYYANSVKG | VKPAYRGGMDV |
| | (SEQ ID NO: 1) | (SEQ ID NO: 7) | (SEQ ID NO: 30) |
| C27-Y2-Y2 | SYAMH | GISYSSASTYYANSVKG | VKVRYRGGMDV |
| | (SEQ ID NO: 1) | (SEQ ID NO: 16) | (SEQ ID NO: 20) |
| COMPOSITE | SYAMH | GISX₁X₂X3X₄STYYANSVKG | X₁X₂X₃X₄YRGGMDV |
| | (SEQ ID NO: 1) | (SEQ ID NO: 78) | (SEQ ID NO: 79) |
| | | wherein | wherein |
| | | X₁ is P, S, H, G or Y; | X₁ is V or S; |
| | | X₂ is S, T, or N; | X₂ is K, F, or R; |
| | | X₃ is G or S; | X₃ is P, V, G, R, S, or L; |
| | | And X₄ is S, V, G, T, A, | And X₄ is G, A, R, K, or L. |
| | | or N. | |
| Antibody | | | |
| Name | LC-CDR1 | LC-CDR2 | LC-CDR3 |
| C27 | RASQSVSSAYLA | GTSRRAT | QLYGSSSVT |
| | (SEQ ID NO: 31) | (SEQ ID NO: 41) | (SEQ ID NO: 42) |
| C27-6-33 | RASQGISSAYLA | GTSRRAT | QLYGATSVT |
| | (SEQ ID NO: 32) | (SEQ ID NO: 41) | (SEQ ID NO: 43) |
| C27-7-33 | RASQGISSAYLA | GTSRRAT | QLYGATSVT |
| | (SEQ ID NO: 32) | (SEQ ID NO: 41) | (SEQ ID NO: 43) |
| C27-24-56 | RASQSVSSAYLA | GTSRRAT | QLYGASSVT |
| | (SEQ ID NO: 31) | (SEQ ID NO: 41) | (SEQ ID NO: 44) |
| C27-47-56 | RASQSVSSAYLA | GTSRRAT | QLYGASSVT |
| | (SEQ ID NO: 31) | (SEQ ID NO: 41) | (SEQ ID NO: 44) |
| C27-33-33 | RASQGISSAYLA | GTSRRAT | QLYGATSVT |
| | (SEQ ID NO: 32) | (SEQ ID NO: 41) | (SEQ ID NO: 43) |
| C27-56-56 | RASQSVSSAYLA | GTSRRAT | QLYGASSVT |
| | (SEQ ID NO: 31) | (SEQ ID NO: 41) | (SEQ ID NO: 44) |
| C27-78-78 | RASQSISTAYLA | GTSRRAT | QLYGASSVT |
| | (SEQ ID NO: 33) | (SEQ ID NO: 41) | (SEQ ID NO: 44) |
| C27-82-58 | RASQDISSAYLA | GTSRRAT | QLYGATSVT |
| | (SEQ ID NO: 34) | (SEQ ID NO: 41) | (SEQ ID NO: 43) |
| C27-54-54 | RASQDVSSAYLA | GTSRRAT | QLYGATSVT |
| | (SEQ ID NO: 35) | (SEQ ID NO: 41) | (SEQ ID NO: 43) |
| C27-36-36 | RASQNISTAYLA | GTSRRAT | QLYGATSVT |
| | (SEQ ID NO: 36) | (SEQ ID NO: 41) | (SEQ ID NO: 43) |
| C27-53-53 | RASQDASNAYLA | GTSRRAT | QLYGSSSVT |
| | (SEQ ID NO: 37) | (SEQ ID NO: 41) | (SEQ ID NO: 42) |
| C27-67-67 | RASQGVSSAYLA | GTSRRAT | QLYGRSSVT |
| | (SEQ ID NO: 38) | (SEQ ID NO: 41) | (SEQ ID NO: 45) |
| C27-55-55 | RASQNISTAYLA | GTSRRAT | QLYGTSSVT |
| | (SEQ ID NO: 36) | (SEQ ID NO: 41) | (SEQ ID NO: 46) |
| C27-59-59 | RASQSVSTAYLA | GTSRRAT | QLYGATSVT |
| | (SEQ ID NO: 39) | (SEQ ID NO: 41) | (SEQ ID NO: 43) |
| C27-58-58 | RASQDISSAYLA | GTSRRAT | QLYGATSVT |
| | (SEQ ID NO: 34) | (SEQ ID NO: 41) | (SEQ ID NO: 43) |
| C27-52-52 | RASQGVSTAYLA | GTSRRAT | QLYGATSVT |
| | (SEQ ID NO: 40) | (SEQ ID NO: 41) | (SEQ ID NO: 43) |
| C27-Y2-Y2 | RASQGVSSAYLA | GTSRRAT | QLYGSTSVT |
| | (SEQ ID NO: 38) | (SEQ ID NO: 41) | (SEQ ID NO: 47) |
| COMPOSITE | RASQX₁X₂SX₃AYLA | GTSRRAT | QLYGX₁X₂SVT |
| | (SEQ ID NO: 80) | (SEQ ID NO: 41) | (SEQ ID NO: 81) |
| | wherein | | wherein |
| | X₁ is G, S, N, or D; | | X₁ is A, S, T, or R; |
| | X₂ is I, V, or A; | | And X₂ is T or S. |
| | And X₃ is S, T, or N. | | |

TABLE 3
Exemplary sequences.

| Description | V_{H} Sequence |
|---|---|
| C27 | EVQLLESGGGLVQPGGSLRLSCAASGFTLSSYAMHWVRQAPGKGLEYVSGISSNGG |
| | STYYANSVKGRFTISRDNPKNTLFLQMSSLRAEDTAVYYCVRVKVGYRGGMDVW |
| | GQGTTVTVSS |
| | (SEQ ID NO: 48) |
| C27-6-33 | EVQLLESGGGLVQPGGSLRLSCAASGFTLSSYAMHWVRQAPGKGLEYVSGISPSGS |
| | STYYANSVKGRFTISRDNPKNTLFLQMSSLRAEDTAVYYCVRSKVRYRGGMDVWG |
| | QGTTVTVSS |
| | (SEQ ID NO: 49) |
| C27-7-33 | EVQLLESGGGLVQPGGSLRLSCAASGFTLSSYAMHWVRQAPGKGLEYVSGISPSGV |
| | STYYANSVKGRFTISRDNPKNTLFLQMSSLRAEDTAVYYCVRVKVKYRGGMDVW |
| | GQGTTVTVSS |
| | (SEQ ID NO: 50) |
| C27-24-56 | EVQLLESGGGLVQPGGSLRLSCAASGFTLSSYAMHWVRQAPGKGLEYVSGISPTSG |
| | STYYANSVKGRFTISRDNPKNTLFLQMSSLRAEDTAVYYCVRVKVRYRGGMDVW |
| | GQGTTVTVSS |
| | (SEQ ID NO: 51) |
| C27-47-56 | EVQLLESGGGLVQPGGSLRLSCAASGFTLSSYAMHWVRQAPGKGLEYVSGISPTGT |
| | STYYANSVKGRFTISRDNPKNTLFLQMSSLRAEDTAVYYCVRVKGAYRGGMDVW |
| | GQGTTVTVSS |
| | (SEQ ID NO: 52) |
| C27-33-33 | EVQLLESGGGLVQPGGSLRLSCAASGFTLSSYAMHWVRQAPGKGLEYVSGISSSGS |
| | STYYANSVKGRFTISRDNPKNTLFLQMSSLRAEDTAVYYCVRVKVAYRGGMDVW |
| | GQGTTVTVSS |
| | (SEQ ID NO: 53) |
| C27-56-56 | EVQLLESGGGLVQPGGSLRLSCAASGFTLSSYAMHWVRQAPGKGLEYVSGISPSSTS |
| | TYYANSVKGRFTISRDNPKNTLFLQMSSLRAEDTAVYYCVRVKVLYRGGMDVWG |
| | QGTTVTVSS |
| | (SEQ ID NO: 54) |
| C27-78-78 | EVQLLESGGGLVQPGGSLRLSCAASGFTLSSYAMHWVRQAPGKGLEYVSGISPSSA |
| | STYYANSVKGRFTISRDNPKNTLFLQMSSLRAEDTAVYYCVRVKSKYRGGMDVWG |
| | QGTTVTVSS |
| | (SEQ ID NO: 55) |
| C27-82-58 | EVQLLESGGGLVQPGGSLRLSCAASGFTLSSYAMHWVRQAPGKGLEYVSGISGNSA |
| | STYYANSVKGRFTISRDNPKNTLFLQMSSLRAEDTAVYYCVRVKLKYRGGMDVWG |
| | QGTTVTVSS |
| | (SEQ ID NO: 56) |
| C27-54-54 | EVQLLESGGGLVQPGGSLRLSCAASGFTLSSYAMHWVRQAPGKGLEYVSGISHSGT |
| | STYYANSVKGRFTISRDNPKNTLFLQMSSLRAEDTAVYYCVRVRVLYRGGMDVWG |
| | QGTTVTVSS |
| | (SEQ ID NO: 57) |
| C27-36-36 | EVQLLESGGGLVQPGGSLRLSCAASGFTLSSYAMHWVRQAPGKGLEYVSGISPSGV |
| | STYYANSVKGRFTISRDNPKNTLFLQMSSLRAEDTAVYYCVRVKVKYRGGMDVW |
| | GQGTTVTVSS |
| | (SEQ ID NO: 50) |
| C27-53-53 | EVQLLESGGGLVQPGGSLRLSCAASGFTLSSYAMHWVRQAPGKGLEYVSGISSNGG |
| | STYYANSVKGRFTISRDNPKNTLFLQMSSLRAEDTAVYYCVRVFVRYRGGMDVWG |
| | QGTTVTVSS |
| | (SEQ ID NO: 58) |
| C27-67-67 | EVQLLESGGGLVQPGGSLRLSCAASGFTLSSYAMHWVRQAPGKGLEYVSGISPTSA |
| | STYYANSVKGRFTISRDNPKNTLFLQMSSLRAEDTAVYYCVRVKGRYRGGMDVW |
| | GQGTTVTVSS |
| | (SEQ ID NO: 59) |
| C27-55-55 | EVQLLESGGGLVQPGGSLRLSCAASGFTLSSYAMHWVRQAPGKGLEYVSGISPTGG |
| | STYYANSVKGRFTISRDNPKNTLFLQMSSLRAEDTAVYYCVRVKGRYRGGMDVW |
| | GQGTTVTVSS |
| | (SEQ ID NO: 60) |
| C27-59-59 | EVQLLESGGGLVQPGGSLRLSCAASGFTLSSYAMHWVRQAPGKGLEYVSGISHSGN |
| | STYYANSVKGRFTISRDNPKNTLFLQMSSLRAEDTAVYYCVRVKRRYRGGMDVWG |
| | QGTTVTVSS |
| | (SEQ ID NO: 61) |
| C27-58-58 | EVQLLESGGGLVQPGGSLRLSCAASGFTLSSYAMHWVRQAPGKGLEYVSGISPSSN |
| | STYYANSVKGRFTISRDNPKNTLFLQMSSLRAEDTAVYYCVRVKVRYRGGMDVW |
| | GQGTTVTVSS |
| | (SEQ ID NO: 62) |
| C27-52-52 | EVQLLESGGGLVQPGGSLRLSCAASGFTLSSYAMHWVRQAPGKGLEYVSGISSSGS |
| | STYYANSVKGRFTISRDNPKNTLFLQMSSLRAEDTAVYYCVRVKPAYRGGMDVWG |
| | QGTTVTVSS |
| | (SEQ ID NO: 63) |
| C27-Y2-Y2 | EVQLLESGGGLVQPGGSLRLSCAASGFTLSSYAMHWVRQAPGKGLEYVSGISYSSA |
| | STYYANSVKGRFTISRDNPKNTLFLQMSSLRAEDTAVYYCVRVKVRYRGGMDVW |
| | GQGTTVTVSS |
| | (SEQ ID NO: 64) |

TABLE 4
Exemplary sequences.

| Description | VL Sequence |
|---|---|
| C27 | ETTLTQSPDTLPLSPGDRASLSCRASQSVSSAYLAWYQQKPGQAPRLLIYGTSRRAT |
| | GVPGRFSGSGSGTDFTLTISRLEPEDFAVYYCQLYGSSSVTFGQGTKLEIK |
| | (SEQ ID NO: 65) |
| C27-6-33 | EIVLTQSPGTLSLSPGERATLSCRASQGISSAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 66) |
| C27-7-33 | EIVLTQSPGTLSLSPGERATLSCRASQGISSAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 66) |
| C27-24-56 | EIVLTQSPGTLSLSPGERATLSCRASQSVSSAYLAWYQQKPGQAPRLLIYGTSRRAT |
| | GIPDRFSGSGSGTDFTLTISRLEPEDFAVYYCQLYGASSVTFGQGTKLEIK |
| | (SEQ ID NO: 67) |
| C27-47-56 | EIVLTQSPGTLSLSPGERATLSCRASQSVSSAYLAWYQQKPGQAPRLLIYGTSRRAT |
| | GIPDRFSGSGSGTDFTLTISRLEPEDFAVYYCQLYGASSVTFGQGTKLEIK |
| | (SEQ ID NO: 67) |
| C27-33-33 | EIVLTQSPGTLSLSPGERATLSCRASQGISSAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 66) |
| C27-56-56 | EIVLTQSPGTLSLSPGERATLSCRASQSVSSAYLAWYQQKPGQAPRLLIYGTSRRAT |
| | GIPDRFSGSGSGTDFTLTISRLEPEDFAVYYCQLYGASSVTFGQGTKLEIK |
| | (SEQ ID NO: 67) |
| C27-78-78 | EIVLTQSPGTLSLSPGERATLSCRASQSISTAYLAWYQQKPGQAPRLLIYGTSRRATG |
| | IPDRFSGSGSGTDFTLTISRLEPEDFAVYYCQLYGASSVTFGQGTKLEIK |
| | (SEQ ID NO: 68) |
| C27-82-58 | EIVLTQSPGTLSLSPGERATLSCRASQDISSAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 69) |
| C27-54-54 | EIVLTQSPGTLSLSPGERATLSCRASQDVSSAYLAWYQQKPGQAPRLLIYGTSRRAT |
| | GIPDRFSGSGSGTDFTLTISRLEPEDFAVYYCQLYGATSVTFGQGTKLEIK |
| | (SEQ ID NO: 70) |
| C27-36-36 | EIVLTQSPGTLSLSPGERATLSCRASQNISTAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 71) |
| C27-53-53 | EIVLTQSPGTLSLSPGERATLSCRASQDASNAYLAWYQQKPGQAPRLLIYGTSRRAT |
| | GIPDRFSGSGSGTDFTLTISRLEPEDFAVYYCQLYGSSSVTFGQGTKLEIK |
| | (SEQ ID NO: 72) |
| C27-67-67 | EIVLTQSPGTLSLSPGERATLSCRASQGVSSAYLAWYQQKPGQAPRLLIYGTSRRAT |
| | GIPDRFSGSGSGTDFTLTISRLEPEDFAVYYCQLYGRSSVTFGQGTKLEIK |
| | (SEQ ID NO: 73) |
| C27-55-55 | EIVLTQSPGTLSLSPGERATLSCRASQNISTAYLAWYQQKPGQAPRLLIYGTSRRATG |
| | IPDRFSGSGSGTDFTLTISRLEPEDFAVYYCQLYGTSSVTFGQGTKLEIK |
| | (SEQ ID NO: 74) |
| C27-59-59 | EIVLTQSPGTLSLSPGERATLSCRASQSVSTAYLAWYQQKPGQAPRLLIYGTSRRAT |
| | GIPDRFSGSGSGTDFTLTISRLEPEDFAVYYCQLYGATSVTFGQGTKLEIK |
| | (SEQ ID NO: 75) |
| C27-58-58 | EIVLTQSPGTLSLSPGERATLSCRASQDISSAYLAWYQQKPGQAPRLLIYGTSRRATG |
| | IPDRFSGSGSGTDFTLTISRLEPEDFAVYYCQLYGATSVTFGQGTKLEIK |
| | (SEQ ID NO: 69) |
| C27-52-52 | EIVLTQSPGTLSLSPGERATLSCRASQGVSTAYLAWYQQKPGQAPRLLIYGTSRRAT |
| | GIPDRFSGSGSGTDFTLTISRLEPEDFAVYYCQLYGATSVTFGQGTKLEIK |
| | (SEQ ID NO: 76) |
| C27-Y2-Y2 | EIVLPQSPGTLSLSPGERATLSCRASQGVSSAYLAWYQQKPGQAPRLLIYGTSRRAT |
| | GIPDRFSGSGSGTDFTLTISRLEPEDFAVYYCQLYGSTSVTFGQGTKLEIK |
| | (SEQ ID NO: 77) |

TABLE 5
Exemplary sequences.

| Description | Sequence |
|---|---|
| Extracellular | MKVLQEPTCVSDYMSISTCEWKMNGPTNCSTELRLLYQLVFLLSEAHTCIPENNGGAGCVCH |
| domain of human | LLMDDVVSADNYTLDLWAGQQLLWKGSFKPSEHVKPRAPGNLTVHTNVSDTLLLTWSNPYP |
| IL-4Rα | PDNYLYNHLTYAVNIWSENDPADFRIYNVTYLEPSLRIAASTLKSGISYRARVRAWAQCYNTT |
| | WSEWSPSTKWHNSYREPFEQH |
| | (SEQ ID NO: 82) |
| complete human | MGWLCSGLLFPVSCLVLLQVASSGNMKVLQEPTCVSDYMSISTCEWKMNGPTNCSTELRLLY |
| IL-4Rα | QLVFLLSEAHTCIPENNGGAGCVCHLLMDDVVSADNYTLDLWAGQQLLWKGSFKPSEHVKP |
| | RAPGNLTVHTNVSDTLLLTWSNPYPPDNYLYNHLTYAVNIWSENDPADFRIYNVTYLEPSLRI |
| | AASTLKSGISYRARVRAWAQCYNTTWSEWSPSTKWHNSYREPFEQHLLLGVSVSCIVILAVCL |
| | LCYVSITKIKKEWWDQIPNPARSRLVAIIIQDAQGSQWEKRSRGQEPAKCPHWKNCLTKLLPC |
| | FLEHNMKRDEDPHKAAKEMPFQGSGKSAWCPVEISKTVLWPESISVVRCVELFEAPVECEEEE |
| | EVEEEKGSFCASPESSRDDFQEGREGIVARLTESLFLDLLGEENGGFCQQDMGESCLLPPSGST |
| | SAHMPWDEFPSAGPKEAPPWGKEQPLHLEPSPPASPTQSPDNLTCTETPLVIAGNPAYRSFSNS |
| | LSQSPCPRELGPDPLLARHLEEVEPEMPCVPQLSEPTTVPQPEPETWEQILRRNVLQHGAAAAP |
| | VSAPTSGYQEFVHAVEQGGTQASAVVGLGPPGEAGYKAFSSLLASSAVSPEKCGFGASSGEEG |
| | YKPFQDLIPGCPGDPAPVPVPLFTFGLDREPPRSPQSSHLPSSSPEHLGLEPGEKVEDMPKPPLP |
| | QEQATDPLVDSLGSGIVYSALTCHLCGHLKQCHGQEDGGQTPVMASPCCGCCCGDRSSPPTT |
| | PLRAPDPSPGGVPLEASLCPASLAPSGISEKSKSSSSFHPAPGNAQSSSQTPKIVNFVSVGPTYM |
| | RVS |
| | (SEQ ID NO: 83) |
| IgG1 heavy chain | ASTKGPSVFPLAPSSKSTSGGTAALGCLVKDYFPEPVTVSWNSGALTSGVHTFPAVL |
| constant region | QSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQP |
| | REPQVYTLPPSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| | (SEQ ID NO: 84) |
| IgG4 heavy chain | ASTKGPSVFPLAPCSRSTSESTAALGCLVKDYFPEPVTVSWNSGALTSGVHTFPAVL |
| constant region | QSSGLYSLSSVVTVPSSSLGTKTYTCNVDHKPSNTKVDKRVESKYGPPCPSCPAPEF |
| | LGGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQ |
| | VYTLPPSQEEMTKNQVSLTCLVKGFYPSDIAVEWESNGQPENNYKTTPPVLDSDGSF |
| | FLYSRLTVDKSRWQEGNVFSCSVMHEALHNHYTQKSLSLSLGK |
| | (SEQ ID NO: 85) |
| Light chain | RTVAAPSVFIFPPSDEQLKSGTASVVCLLNNFYPREAKVQWKVDNALQSGNSQESV |
| constant region | TEQDSKDSTYSLSSTLTLSKADYEKHKVYACEVTHQGLSSPVTKSFNRGEC |
| | (SEQ ID NO: 86) |

IL-4 AND IL-13

IL-4 and IL-13 are the signature cytokines of the type II inflammatory response. They are key players in the inflammatory response triggered by either an invading parasite or allergen. They regulate many aspects of allergic inflammation and play important roles in regulating the responses of lymphocytes, myeloid cells, and non-hematopoietic cells. In T-cells, IL-4 induces the differentiation of naïve CD4 T cells into Th2 cells, in B cells, IL-4 drives the immunoglobulin (Ig) class switch to IgG1 and IgE, and in macrophages, IL-4 and IL-13 induce alternative macrophage activation. The cellular sources of IL-4 and IL-13 have been studied extensively and along with CD4 T cells, basophils, eosinophils, mast cells, and NK T cells, appropriately stimulated ILC2 cells have the ability to produce IL-4 and IL-13 (Ilkka S. Junttila, Tuning the Cytokine Responses: An Update on Interleukin (IL)-4 and IL-13 Receptor Complexes, Front Immunol. 2018; 9: 888).

The genomic locus, where IL-4 and IL-13 are produced (along with IL-5), is called the Th2 cytokine locus, which is located on chromosome 5 in humans and on chromosome 11 in mice and is under the control of the locus control region (LCR) of the Rad 50 gene (Zhu J. T helper 2 (T2) cell differentiation, type 2 innate lymphoid cell (ILC2) development and regulation of interleukin-4 (IL-4) and IL-13 production. Cytokine (2015) 75:14-24; Ansel K M, et al. Regulation of T2 differentiation and IL4 locus accessibility. Annu Rev Immunol (2006) 24:607-56). The LCR in CD4 T-cells is indispensable for the production of IL-4 and IL-13 in vivo (Koh B H, et al. T2 LCR is essential for regulation of T2 cytokine genes and for pathogenesis of allergic asthma. Proc Natl Acad Sci USA (2010) 107:10614-9). The production of the two cytokines is not identical though: IL-4 production is calcineurin dependent, whereas IL-13 production is only partially dependent on calcineurin (Guo L, et al. Elevating calcium in T2 cells activates multiple pathways to induce IL-4 transcription and mRNA stabilization. J Immunol (2008) 181:3984-93). Upon the appropriate stimulation of the cells, the LCR of the Th2 cytokine locus is epigenetically modified to allow the access of transcription factors to the DNA and the subsequent transcription of these cytokines. This complex regulation was recently reviewed in detail (Zhu J. T helper 2 (T2) cell differentiation, type 2 innate lymphoid cell (ILC2) development and regulation of interleukin-4 (IL-4) and IL-13 production. Cytokine (2015) 75:14-24). Interestingly and in line with findings in mice, a polymorphism in the murine equivalent of the DNase I hypersensitive site (RHS)7 in humans affects DNA methylation and gene expression at 5q31 and subsequently IgE levels on a population level (Schieck M, et al. A polymorphism in the TH 2 locus control region is associated with changes in DNA methylation and gene expression. Allergy (2014) 69:1171-80).

IL-4 Receptor System

When IL-4 or IL-13 is released from T cells, cells carrying the receptors for these cytokines will respond. For IL-4 and IL-13, the unique utilization of the STATE transcription factor in the signaling they elicit allows them to execute specific functions on different cell types; IL-4 is the regulator of lymphocyte functions (Th2 differentiation and B-cell IgG1 and IgE class switch), whereas IL-13 is an effector cytokine, regulating smooth cell muscle contraction and mucus production in the airway epithelium, for example, in allergic asthma (Wills-Karp M, et al. Interleukin-13: central mediator of allergic asthma. Science (1998) 282:2258-61).

The cytokine-binding receptor chain for IL-4 is IL-4Rα. Upon IL-4 binding to IL-4Rα, the IL-4/IL-4Rα-complex will bind a secondary receptor chain, either IL-2Rγc (γc) or IL-13Rα1. The expression of these secondary chains varies among different cell types. In non-hematopoietic cells, γc expression is low or absent, whereas higher amounts of IL-13Rα1 are expressed in these cells. By contrast, lymphocytes express only low levels of IL-13Rα1 and relatively large amounts of γc. Finally, myeloid cells fall in between non-hematopoietic cells and lymphocytes, as they express both IL-13Rα1 and γc.

Interleukin-4 and IL-13 regulate cellular functions and activate transcriptional machinery via cell surface receptors. For IL-4, binding of the cytokine to a single cell surface receptor chain (IL-4Rα) generates a ligand/receptor complex that requires the recruitment of a third receptor chain to form a functional receptor complex. The receptor formed by IL-4/IL-4Rα with γc is a type I IL-4 receptor and the IL-4/IL-4Rα complex binding IL-13Rα1 is a type II IL-4 receptor (Nelms K, et al. The IL-4 receptor: signaling mechanisms and biologic functions. Annu Rev Immunol (1999) 17:701-38). Thus, based on their tissue distribution, the type I IL-4 receptor is found in lymphocytes and myeloid cells, and the type II IL-4 receptor is expressed in myeloid cells and all non-hematopoietic cells. The binding of IL-4 to IL-4Rα occurs with high affinity (Kd in the order of 10-10 M). This effectively means that at very low concentrations of IL-4 it can maximally occupy the receptor chains at a given cell surface.

Allergic inflammation is an inappropriately controlled inflammatory response with characteristic hallmarks of eosinophilia, elevated immunoglobulin (Ig)E-levels, increased mucus production, and typical cytokine/chemokine expression. Clinically, these basic pathophysiological mechanisms result in symptoms varying from mild skin rash (atopic dermatitis) and runny nose (allergic rhinitis) to life-threatening problems in breathing (allergic asthma). This inflammatory process from the very initiation is critically regulated by cytokines and chemokines. The cytokines regulate cellular responses at transcriptional level, while chemokines play a role in recruiting inflammatory cells to the sites on inflammation. One of the central cytokines regulating allergic inflammation is interleukin (IL)-4 and since its cloning, efforts targeting IL-4 have been made to decrease IL-4-induced inflammation. In part, these efforts have been slowed down by the receptor of IL-4, which is ubiquitously expressed and easily saturated by the ligand. Pathologically, IL-4 has been shown to play a role in exacerbating inflammatory process and allergic inflammation. Neutralization of IL-4 binding to IL-4Rα is therefore a therapeutic approach to treating diseases and conditions mediated through IL-4Rα.

Full-Length Anti-IL-4Rα Antibody

The anti-IL-4Rα antibody in some embodiments is a full-length anti-IL-4Rα antibody. In some embodiments, the full-length anti-IL-4Rα antibody is an IgA, IgD, IgE, IgG, or IgM. In some embodiments, the full-length anti-IL-4Rα antibody comprises IgG constant domains, such as constant domains of any one of IgG1, IgG2, IgG3, and IgG4 including variants thereof. In some embodiments, the full-length anti-IL-4Rα antibody comprises a lambda light chain constant region. In some embodiments, the full-length anti-IL-4Rα antibody comprises a kappa light chain constant region. In some embodiments, the full-length anti-IL-4Rα antibody is a full-length human anti-IL-4Rα antibody. In some embodiments, the full-length anti-IL-4Rα antibody comprises an Fc sequence of a mouse immunoglobulin. In some embodiments, the full-length anti-IL-4Rα antibody comprises an Fc sequence that has been altered or otherwise changed so that it has enhanced antibody dependent cellular cytotoxicity (ADCC) or complement dependent cytotoxicity (CDC) effector function.

Thus, for example, in some embodiments, there is provided a full-length anti-IL-4Rα antibody comprising IgG1 constant domains, wherein the anti-IL-4Rα antibody specifically binds to IL-4Rα. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG2 constant domains, wherein the anti-IL-4Rα antibody specifically binds to IL-4Rα. In some embodiments, the IgG2 is human IgG2. In some embodiments, the light chain constant region comprises or consists of the amino acid sequence of SEQ ID NO: 86.

In some embodiments, there is provided a full-length anti-IL-4Rα antibody comprising IgG3 constant domains, wherein the anti-IL-4Rα antibody specifically binds to IL-4Rα. In some embodiments, the IgG3 is human IgG3. In some embodiments, the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody specifically binds to IL-4Rα. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG2 constant domains, wherein the anti-IL-4Rα antibody comprises a) a heavy chain variable domain comprising an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions. In some embodiments, the IgG2 is human IgG2. In some embodiments, the light chain constant region comprises or consists of the amino acid sequence of SEQ ID NO: 86.

In some embodiments, there is provided a full-length anti-IL-4Rα antibody comprising IgG3 constant domains, wherein the anti-IL-4Rα antibody comprises a) a heavy chain variable domain comprising an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions. In some embodiments, the IgG3 is human IgG3. In some embodiments, the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, or a variant thereof comprising up to about 3 (such as about any of 1, 2, or 3) amino acid substitutions, an HC-CDR2 comprising the amino acid sequence of any one of SEQ ID NOs: 2-16, or a variant thereof comprising up to about 3 (such as about any of 1, 2, or 3) amino acid substitutions, and an HC-CDR3 comprising the amino acid sequence of any one of SEQ ID NOs: 17-30, or a variant thereof comprising up to about 3 (such as about any of 1, 2, or 3) amino acid substitutions; and b) a light chain variable domain comprising an LC-CDR1 comprising the amino acid sequence of any one of SEQ ID NOs: 31-40, or a variant thereof comprising up to about 3 (such as about any of 1, 2, or 3) amino acid substitutions, an LC-CDR2 comprising the amino acid sequence of SEQ ID NO: 41, or a variant thereof comprising up to about 3 (such as about any of 1, 2, or 3) amino acid substitutions, and an LC-CDR3 comprising the amino acid sequence of any one of SEQ ID NOs: 42-47, or a variant thereof comprising up to about 3 (such as about any of 1, 2, or 3) amino acid substitutions. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30, or a variant thereof comprising up to about 5 (such as about any of 1, 2, 3, 4 or 5) amino acid substitutions in the HC-CDR sequences; and b) a light chain variable domain comprising an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47, or a variant thereof comprising up to about 5 (such as about any of 1, 2, 3, 4 or 5) amino acid substitutions in the LC-CDR sequences. In some embodiments, the IgG1 is human IgG1. In some embodiments, the anti-IL-4Rα heavy chain constant region comprises or consists of the amino acid sequence of SEQ ID NO: 84. In some embodiments, the anti-IL-4Rα light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30, or a variant thereof comprising up to about 5 (such as about any of 1, 2, 3, 4 or 5) amino acid substitutions in the HC-CDR sequences; and b) a light chain variable domain comprising an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47, or a variant thereof comprising up to about 5 (such as about any of 1, 2, 3, 4 or 5) amino acid substitutions in the LC-CDR sequences. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30; and b) a light chain variable domain comprising an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30; and b) a light chain variable domain comprising an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24; and b) a light chain variable domain comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25; and b) a light chain variable domain comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26; and b) a light chain variable domain comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and b) a light chain variable domain comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27; and b) a light chain variable domain comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28; and b) a light chain variable domain comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28; and b) a light chain variable domain comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29; and b) a light chain variable domain comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20; and b) a light chain variable domain comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30; and b) a light chain variable domain comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20; and b) a light chain variable domain comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22; and b) a light chain variable domain comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23; and b) a light chain variable domain comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24; and b) a light chain variable domain comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25; and b) a light chain variable domain comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26; and b) a light chain variable domain comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19; and b) a light chain variable domain comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27; and b) a light chain variable domain comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28; and b) a light chain variable domain comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28; and b) a light chain variable domain comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29; and b) a light chain variable domain comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20; and b) a light chain variable domain comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30; and b) a light chain variable domain comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a) a heavy chain variable domain comprising an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20; and b) a light chain variable domain comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of any one of SEQ ID NOs: 48-64, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 65-77, or a variant thereof having at least about 90% (for example at least about any of 91%, 92%, 93%, 94%, 95%, 96%, 97%, 98%, or 99%) sequence identity. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG2 constant domains, wherein the anti-IL-4Rα antibody comprises a heavy chain variable domain comprising the amino acid sequence of any one of SEQ ID NOs: 48-64, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 65-77, or a variant thereof having at least about 90% (for example at least about any of 91%, 92%, 93%, 94%, 95%, 96%, 97%, 98%, or 99%) sequence identity. In some embodiments, the IgG2 is human IgG2. In some embodiments, the light chain constant region comprises or consists of the amino acid sequence of SEQ ID NO: 86.

In some embodiments, there is provided a full-length anti-IL-4Rα antibody comprising IgG3 constant domains, wherein the anti-IL-4Rα antibody comprises a heavy chain variable domain comprising the amino acid sequence of any one of SEQ ID NOs: 48-64, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 65-77, or a variant thereof having at least about 90% (for example at least about any of 91%, 92%, 93%, 94%, 95%, 96%, 97%, 98%, or 99%) sequence identity. In some embodiments, the IgG3 is human IgG3. In some embodiments, the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of any one of SEQ ID NOs: 48-64, or a variant thereof having at least about 90% (for example at least about any of 91%, 92%, 93%, 94%, 95%, 96%, 97%, 98%, or 99%) sequence identity, and a light chain variable domain comprising the amino acid sequence of any one of SEQ ID NOs: 65-77, or a variant thereof having at least about 90% (for example at least about any of 91%, 92%, 93%, 94%, 95%, 96%, 97%, 98%, or 99%) sequence identity. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO:85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of any one of SEQ ID NOs: 48-64, and a light chain variable domain comprising the amino acid sequence of any one of SEQ ID NOs: 65-77. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of any one of SEQ ID NOs: 48-64, and a light chain variable domain comprising the amino acid sequence of any one of SEQ ID NOs: 65-77. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO:85 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 48 and a light chain variable domain comprising the amino acid sequence of SEQ ID NO: 65. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 49 and a light chain variable domain comprising the amino acid sequence of SEQ ID NO: 66. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 50 and a light chain variable domain comprising the amino acid sequence of SEQ ID NO: 66. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 51 and a light chain variable domain comprising the amino acid sequence of SEQ ID NO: 67. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 52 and a light chain variable domain comprising the amino acid sequence of SEQ ID NO: 67. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 53 and a light chain variable domain comprising the amino acid sequence of SEQ ID NO: 66. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 54 and a light chain variable domain comprising the amino acid sequence of SEQ ID NO: 67. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 55 and a light chain variable domain comprising the amino acid sequence of SEQ ID NO: 68. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 56 and a light chain variable domain comprising the amino acid sequence of SEQ ID NO: 69. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 57 and a light chain variable domain comprising the amino acid sequence of SEQ ID NO: 70. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 50 and a light chain variable domain comprising the amino acid sequence of SEQ ID NO: 71. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 58 and a light chain variable domain comprising the amino acid sequence of SEQ ID NO: 72. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 59 and a light chain variable domain comprising the amino acid sequence of SEQ ID NO: 73. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 60 and a light chain variable domain comprising the amino acid sequence of SEQ ID NO: 74. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 61 and a light chain variable domain comprising the amino acid sequence of SEQ ID NO: 75. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 62 and a light chain variable domain comprising the amino acid sequence of SEQ ID NO: 69. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 63 and a light chain variable domain comprising the amino acid sequence of SEQ ID NO: 76. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG1 constant domains, wherein the anti-IL-4Rα antibody comprises a heavy chain variable domain comprising the amino acid sequence of SEQ ID NO: 64 and a light chain variable domain comprising the amino acid sequence of SEQ ID NO: 77. In some embodiments, the IgG1 is human IgG1. In some embodiments, the heavy chain constant region comprises or consists of the amino acid sequence of SEQ ID NO: 84. In some embodiments, the light chain constant region comprises or consists of the amino acid sequence of SEQ ID NO: 86. In some embodiments, the heavy chain constant region comprises or consists of the amino acid sequence of SEQ ID NO: 84 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 48 and a light chain variable domain comprising the amino acid sequence of SEQ ID NO: 65. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 49 and a light chain variable domain comprising the amino acid sequence of SEQ ID NO: 66. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 50 and a light chain variable domain comprising the amino acid sequence of SEQ ID NO: 66. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 51 and a light chain variable domain comprising the amino acid sequence of SEQ ID NO: 67. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 52 and a light chain variable domain comprising the amino acid sequence of SEQ ID NO: 67. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 53 and a light chain variable domain comprising the amino acid sequence of SEQ ID NO: 66. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 54 and a light chain variable domain comprising the amino acid sequence of SEQ ID NO: 67. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 55 and a light chain variable domain comprising the amino acid sequence of SEQ ID NO: 68. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 56 and a light chain variable domain comprising the amino acid sequence of SEQ ID NO: 69. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 57 and a light chain variable domain comprising the amino acid sequence of SEQ ID NO: 70. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 50 and a light chain variable domain comprising the amino acid sequence of SEQ ID NO: 71. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 58 and a light chain variable domain comprising the amino acid sequence of SEQ ID NO: 72. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 59 and a light chain variable domain comprising the amino acid sequence of SEQ ID NO: 73. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 60 and a light chain variable domain comprising the amino acid sequence of SEQ ID NO: 74. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 61 and a light chain variable domain comprising the amino acid sequence of SEQ ID NO: 75. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 62 and a light chain variable domain comprising the amino acid sequence of SEQ ID NO: 69. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 63 and a light chain variable domain comprising the amino acid sequence of SEQ ID NO: 76. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

In some embodiments, there is provided a full-length anti-IL-4Rα antibody comprising IgG4 constant domains, wherein the anti-IL-4Rα antibody comprises a heavy chain variable domain comprising the amino acid sequence of SEQ ID NO: 64 and a light chain variable domain comprising the amino acid sequence of SEQ ID NO: 77. In some embodiments, the IgG4 is human IgG4. In some embodiments, the heavy chain constant region comprises or consists of the amino acid sequence of SEQ ID NO: 85. In some embodiments, the light chain constant region comprises or consists of the amino acid sequence of SEQ ID NO: 86. In some embodiments, the heavy chain constant region comprises or consists of the amino acid sequence of SEQ ID NO: 85 and the light chain constant region comprises or consists of the amino acid sequence of SEQ ID NO: 86.

Binding Affinity

Binding affinity can be indicated by Kd, Koff, Kon, or Ka. The term “Koff”, as used herein, is intended to refer to the off-rate constant for dissociation of an antibody from the antibody/antigen complex, as determined from a kinetic selection set up. The term “Kon”, as used herein, is intended to refer to the on-rate constant for association of an antibody to the antigen to form the antibody/antigen complex. The term dissociation constant “Kd”, as used herein, refers to the dissociation constant of a particular antibody-antigen interaction, and describes the concentration of antigen required to occupy one half of all of the antibody-binding domains present in a solution of antibody molecules at equilibrium, and is equal to Koff/Kon. The measurement of Kd presupposes that all binding agents are in solution. In the case where the antibody is tethered to a cell wall, e.g., in a yeast expression system, the corresponding equilibrium rate constant is expressed as EC50, which gives a good approximation of Kd. The affinity constant, Ka, is the inverse of the dissociation constant, Kd.

The dissociation constant (Kd) is used as an indicator showing affinity of antibody moieties to antigens. For example, easy analysis is possible by the Scatchard method using antibodies marked with a variety of marker agents, as well as by using Biacore (made by Amersham Biosciences), analysis of biomolecular interactions by surface plasmon resonance, according to the user's manual and attached kit. The Kd value that can be derived using these methods is expressed in units of M. An antibody that specifically binds to a target may have a Kd of, for example, ≤10⁻⁷M, ≤10⁻⁸M, ≤10⁻⁹ M, ≤10⁻¹⁰ M, ≤10⁻¹¹ M, ≤10⁻¹² M, or ≤10⁻¹³ M.

Binding specificity of the antibody can be determined experimentally by methods known in the art. Such methods comprise, but are not limited to, Western blots, ELISA-, RIA-, ECL-, IRMA-, EIA-, BIAcore-tests and peptide scans.

In some embodiments, the anti-IL-4Rα antibody specifically binds to a target IL-4Rα with a Kd of about 10⁻⁷M to about 10⁻¹³ M (such as about 10⁻⁷ M to about 10⁻¹³ M, about 10⁻⁸ M to about 10⁻¹³ M, about 10⁻⁹ M to about 10⁻¹³ M, or about 10⁻¹⁰ M to about 10⁻¹² M). Thus in some embodiments, the Kd of the binding between the anti-IL-4Rα antibody and IL-4Rα, is about 10⁻⁷M to about 10⁻¹³ M, about 1×10⁻⁷M to about 5×10⁻¹³ M, about 10⁻⁷M to about 10⁻¹² M about 10⁻⁷M to about 10⁻¹¹M, about 10⁻⁷M to about 10⁻¹⁰ M, about 10⁻⁷M to about 10⁻⁹M, about 10⁻⁸M to about 10⁻¹³ M, about 1×10⁻⁸M to about 5×10⁻¹³ M, about 10⁻⁸M to about 10⁻¹² M about 10⁻⁸M to about 10⁻¹¹M, about 10⁻⁸M to about 10⁻¹⁰ M, about 10⁻⁸M to about 10⁻⁹ M, about 5×10⁻⁹M to about 1×10⁻¹³M, about 5×10⁻⁹M to about 1×10⁻¹²M, about 5×10⁻⁹M to about 1×10⁻¹¹M, about 5×10⁻⁹M to about 1×10⁻¹⁰ M, about 10⁻⁹ M to about 10⁻¹³M, about 10⁻⁹ M to about 10⁻¹²M, about 10⁻⁹ M to about 10⁻¹¹M, about 10⁻⁹M to about 10⁻¹⁰ M, about 5×10⁻¹⁰ M to about 1×10⁻¹³M, about 5×10⁻¹⁰ M to about 1×10⁻¹² M, about 5×10⁻¹⁰ M to about 1×10⁻¹¹M, about 10⁻¹⁰ M to about 10⁻¹³ M, about 1×10⁻¹⁰ M to about 5×10⁻¹³M, about 1×10⁻¹⁰ M to about 1×10⁻¹²M, about 1×10⁻¹⁰ M to about 5×10⁻¹²M, about 1×10⁻¹⁰ M to about 1×10⁻¹¹ M, about 10⁻¹¹M to about 10⁻¹³M, about 1×10⁻¹¹ M to about 5×10⁻¹³ M, about 10⁻¹¹M to about 10⁻¹²M, or about 10⁻¹² M to about 10⁻¹³M. In some embodiments, the Kd of the binding between the anti-IL-4Rα antibody and a IL-4Rα is about 10⁻⁷ M to about 10⁻¹³ M.

In some embodiments, the Kd of the binding between the anti-IL-4Rα antibody and a non-target is more than the Kd of the binding between the anti-IL-4Rα antibody and the target, and is herein referred to in some embodiments as the binding affinity of the anti-IL-4Rα antibody to the target (e.g., IL-4Rα) is higher than that to a non-target. In some embodiments, the non-target is an antigen that is not IL-4Rα. In some embodiments, the Kd of the binding between the anti-IL-4Rα antibody (against IL-4Rα) and a non-IL-4Rα target can be at least about 10 times, such as about 10⁻¹⁰⁰ times, about 100-1000 times, about 10³-10⁴ times, about 10⁴-10⁵ times, about 10⁵-10⁶ times, about 10⁶-10⁷ times, about 10⁷-10⁸ times, about 10⁸-10⁹ times, about 10⁹-10¹⁰ times, about 10¹⁰-10¹¹ times, or about 10¹¹-10¹² times of the Kd of the binding between the anti-IL-4Rα antibody and a target IL-4Rα.

In some embodiments, the anti-IL-4Rα antibody binds to a non-target with a Kd of about 10⁻¹M to about 10⁻⁶ M (such as about 10⁻¹ M to about 10⁻⁶ M, about 10⁻¹ M to about 10⁻⁵ M, or about 10⁻² M to about 10⁻⁴M). In some embodiments, the non-target is an antigen that is not IL-4Rα. Thus in some embodiments, the Kd of the binding between the anti-IL-4Rα antibody and a non-IL-4Rα target is about 10⁻¹M to about 10⁻⁶ M, about 1×10⁻¹M to about 5×10⁻⁶M, about 10⁻¹M to about 10⁻⁵M, about 1×10⁻¹M to about 5×10⁻⁵ M, about 10⁻¹M to about 10⁻⁴M, about 1×10⁻¹M to about 5×10⁻⁴M, about 10⁻¹ M to about 10⁻³M, about 1×10⁻¹ M to about 5×10⁻³ M, about 10⁻¹M to about 10⁻²M, about 10⁻²M to about 10⁻⁶M, about 1×10⁻²M to about 5×10⁻⁶ M, about 10⁻²M to about 10⁻⁵ M, about 1×10⁻²M to about 5×10⁻⁵M, about 10⁻²M to about 10⁻⁴M, about 1×10⁻²M to about 5×10⁻⁴M, about 10⁻²M to about 10⁻³ M, about 10⁻³M to about 10⁻⁶ M, about 1×10⁻³M to about 5×10⁻⁶ M, about 10⁻³ M to about 10⁻⁵ M, about 1×10⁻³M to about 5×10⁻⁵ M, about 10⁻³M to about 10⁻⁴M, about 10⁻⁴M to about 10⁻⁶ M, about 1×10⁻⁴M to about 5×10⁻⁶ M, about 10⁻⁴ M to about 10⁻⁵M, or about 10⁻⁵ M to about 10⁻⁶ M.

In some embodiments, when referring to that the anti-IL-4Rα antibody specifically recognizes a target IL-4Rα at a high binding affinity, and binds to a non-target at a low binding affinity, the anti-IL-4Rα antibody will bind to the target IL-4Rα with a Kd of about 10⁻⁷M to about 10⁻¹³ M (such as about 10⁻⁷M to about 10⁻¹³M, about 10⁻⁸M to about 10⁻¹³M, about 10⁻⁹ M to about 10⁻¹³ M, or about 10⁻¹⁰ M to about 10⁻¹²M), and will bind to the non-target with a Kd of about 10⁻¹M to about 10⁻⁶ M (such as about 10⁻¹ M to about 10⁻⁶ M, about 10⁻¹ M to about 10⁻⁵ M, or about 10⁻²M to about 10⁻⁴M).

In some embodiments, when referring to that the anti-IL-4Rα antibody specifically recognizes IL-4Rα, the binding affinity of the anti-IL-4Rα antibody is compared to that of a control anti-IL-4Rα antibody. In some embodiments, the Kd of the binding between the control anti-IL-4Rα antibody and IL-4Rα can be at least about 2 times, such as about 2 times, about 3 times, about 4 times, about 5 times, about 6 times, about 7 times, about 8 times, about 9 times, about 10 times, about 10⁻¹⁰⁰ times, about 100-1000 times, about 10³-10⁴ times of the Kd of the binding between the anti-IL-4Rα antibody described herein and IL-4Rα.

Nucleic Acids

Nucleic acid molecules encoding the anti-IL-4Rα antibodies are also contemplated. In some embodiments, there is provided a nucleic acid (or a set of nucleic acids) encoding a full-length anti-IL-4Rα antibody, including any of the full-length anti-IL-4Rα antibodies described herein. In some embodiments, the nucleic acid (or a set of nucleic acids) encoding the anti-IL-4Rα antibody described herein may further comprises a nucleic acid sequence encoding a peptide tag (such as protein purification tag, e.g., His-tag, HA tag).

Also contemplated here are isolated host cells comprising an anti-IL-4Rα antibody, an isolated nucleic acid encoding the polypeptide components of the anti-IL-4Rα antibody, or a vector comprising a nucleic acid encoding the polypeptide components of the anti-IL-4Rα antibody described herein.

The present application also includes variants to these nucleic acid sequences. For example, the variants include nucleotide sequences that hybridize to the nucleic acid sequences encoding the anti-IL-4Rα antibodies of the present application under at least moderately stringent hybridization conditions.

The present application also provides vectors in which a nucleic acid of the present application is inserted.

In brief summary, the expression of an anti-IL-4Rα antibody (e.g., full-length anti-IL-4Rα antibody) by a natural or synthetic nucleic acid encoding the anti-IL-4Rα antibody can be achieved by inserting the nucleic acid into an appropriate expression vector, such that the nucleic acid is operably linked to 5′ and 3′ regulatory elements, including for example a promoter (e.g., a lymphocyte-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequences.

The nucleic acids of the present application may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In some embodiments, the application provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Green and Sambrook (2013, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the application should not be limited to the use of constitutive promoters. Inducible promoters are also contemplated as part of the application. The use of an inducible promoter provides a molecular switch capable of turning on expression of the polynucleotide sequence to which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the anti-IL-4Rα antibody is inducible. In some embodiments, a nucleic acid sequence encoding the anti-IL-4Rα antibody is operably linked to an inducible promoter, including any inducible promoter described herein.

Inducible Promoters

The use of an inducible promoter provides a molecular switch capable of turning on expression of the polynucleotide sequence to which it is operatively linked when such expression is desired, or turning off the expression when expression is not desired. Exemplary inducible promoter systems for use in eukaryotic cells include, but are not limited to, hormone-regulated elements (e.g., see Mader, S. and White, J. H. (1993) Proc. Natl. Acad. Sci. USA 90:5603-5607), synthetic ligand-regulated elements (see, e.g., Spencer, D. M. et al 1993) Science 262: 1019-1024) and ionizing radiation-regulated elements (e.g., see Manome, Y. et al. (1993) Biochemistry 32: 10607-10613; Datta, R. et al. (1992) Proc. Natl. Acad. Sci. USA 89: 1014-10153). Further exemplary inducible promoter systems for use in in vitro or in vivo mammalian systems are reviewed in Gingrich et al. (1998) Annual Rev. Neurosci 21:377-405. In some embodiments, the inducible promoter system for use to express the anti-IL-4Rα antibody is the Tet system. In some embodiments, the inducible promoter system for use to express the anti-IL-4Rα antibody is the lac repressor system from E. coli.

An exemplary inducible promoter system for use in the present application is the Tet system. Such systems are based on the Tet system described by Gossen et al. (1993). In an exemplary embodiment, a polynucleotide of interest is under the control of a promoter that comprises one or more Tet operator (TetO) sites. In the inactive state, Tet repressor (TetR) will bind to the TetO sites and repress transcription from the promoter. In the active state, e.g., in the presence of an inducing agent such as tetracycline (Tc), anhydrotetracycline, doxycycline (Dox), or an active analog thereof, the inducing agent causes release of TetR from TetO, thereby allowing transcription to take place. Doxycycline is a member of the tetracycline family of antibiotics with the chemical name of 1-dimethylamino-2,4a,5,7,12-pentahydroxy-11-methyl-4,6-dioxo-1,4a,11,11a,12,12a-hexahydrotetracene-3-carboxamide.

In one embodiment, a TetR is codon-optimized for expression in mammalian cells, e.g., murine or human cells. Most amino acids are encoded by more than one codon due to the degeneracy of the genetic code, allowing for substantial variations in the nucleotide sequence of a given nucleic acid without any alteration in the amino acid sequence encoded by the nucleic acid. However, many organisms display preference in codon usage, also known as “codon bias” (i.e., bias for use of a particular codon(s) for a given amino acid). Codon bias often correlates with the presence of a predominant species of tRNA for a particular codon, which in turn increases efficiency of mRNA translation. Accordingly, a coding sequence derived from a particular organism (e.g., a prokaryote) may be tailored for improved expression in a different organism (e.g., a eukaryote) through codon optimization.

Other specific variations of the Tet system include the following “Tet-Off” and “Tet-On” systems. In the Tet-Off system, transcription is inactive in the presence of Tc or Dox. In that system, a tetracycline-controlled transactivator protein (tTA), which is composed of TetR fused to the strong transactivating domain of VP16 from Herpes simplex virus, regulates expression of a target nucleic acid that is under transcriptional control of a tetracycline-responsive promoter element (TRE). The TRE is made up of TetO sequence concatamers fused to a promoter (commonly the minimal promoter sequence derived from the human cytomegalovirus (hCMV) immediate-early promoter). In the absence of Tc or Dox, tTA binds to the TRE and activates transcription of the target gene. In the presence of Tc or Dox, tTA cannot bind to the TRE, and the expression of the target gene remains inactive.

Conversely, in the Tet-On system, transcription is active in the presence of Tc or Dox. The Tet-On system is based on a reverse tetracycline-controlled transactivator, rtTA. Like tTA, rtTA is a fusion protein comprised of the TetR repressor and the VP16 transactivation domain. However, a four-amino-acids change in the TetR DNA binding moiety alters rtTA's binding characteristics so that it can only recognize the tet0 sequences in the TRE of the target transgene in the presence of Dox. Thus, in the Tet-On system, transcription of the TRE-regulated target gene is stimulated by rtTA only in the presence of Dox.

Another inducible promoter system available is the lac repressor system from E. coli (See Brown et al., Cell 49:603-612 (1987)). The lac repressor system functions by regulating transcription of a polynucleotide of interest operably linked to a promoter comprising the lac operator (lacO). The lac repressor (lacR) binds to LacO, thus preventing transcription of the polynucleotide of interest. Expression of the polynucleotide of interest is induced by a suitable inducing agent, e.g., isopropyl-β-D-thiogalactopyranoside (IPTG).

In order to assess the expression of a polypeptide or portions thereof, the expression vector to be introduced into a cell can also contain either a selectable marker gene or a reporter gene or both to facilitate identification and selection of target cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, there is provided nucleic acid encoding a full-length anti-IL-4Rα antibody according to any of the full-length anti-IL-4Rα antibodies described herein. In some embodiments, the nucleic acid comprises one or more nucleic acid sequences encoding the heavy and light chains of the full-length anti-IL-4Rα antibody. In some embodiments, each of the one or more nucleic acid sequences are contained in separate vectors. In some embodiments, at least some of the nucleic acid sequences are contained in the same vector. In some embodiments, all of the nucleic acid sequences are contained in the same vector. Vectors may be selected, for example, from the group consisting of mammalian expression vectors and viral vectors (such as those derived from retroviruses, adenoviruses, adeno-associated viruses, herpes viruses, and lentiviruses).

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Green and Sambrook (2013, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape.

Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell or otherwise expose a cell to the inhibitor of the present application, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the application.

Preparation of Anti-IL-4Rα Antibodies

In some embodiments, the anti-IL-4Rα antibody is a monoclonal antibody or derived from a monoclonal antibody. In some embodiments, the anti-IL-4Rα antibody comprises V_{H} and V_{L} domains, or variants thereof, from a monoclonal antibody. In some embodiments, the anti-IL-4Rα antibody further comprises C_{H}1 and C_{L} domains, or variants thereof, from a monoclonal antibody. Monoclonal antibodies can be prepared, e.g., using known methods in the art, including hybridoma methods, phage display methods, or using recombinant DNA methods. Additionally, exemplary phage display methods are described herein and in the Examples below.

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunosorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub-cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub-clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In some embodiments, according to any of the anti-IL-4Rα antibodies described herein, the anti-IL-4Rα antibody comprises sequences from a clone selected from an antibody library (such as a phage library presenting scFv or Fab fragments). The clone may be identified by screening combinatorial libraries for antibody fragments with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of both non-self and self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The anti-IL-4Rα antibodies can be prepared by phage display to screen libraries for anti-IL-4Rα antibody moieties specific to the target IL-4Rα. The library can be a human scFv phage display library having a diversity of at least 1×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a fully-synthetic phage display library. In some embodiments, the library is a non-human phage display library.

Phage clones that bind to the target IL-4Rα with high affinity can be selected by iterative binding of phage to the target IL-4Rα, which is bound to a solid support (such as, for example, beads for solution panning or mammalian cells for cell panning), followed by removal of non-bound phage and by elution of specifically bound phage. The bound phage clones are then eluted and used to infect an appropriate host cell, such as E. coli XL1-Blue, for expression and purification. The panning can be performed for multiple (such as about any of 2, 3, 4, 5, 6 or more) rounds with solution panning, cell panning, or a combination of both, to enrich for phage clones binding specifically to the target IL-4Rα. Enriched phage clones can be tested for specific binding to the target IL-4Rα by any methods known in the art, including for example ELISA and FACS.

Monoclonal antibodies can also be made by recombinant DNA methods, such as those described in U.S. Pat. No. 4,816,567. DNA encoding the monoclonal antibodies of the application can be readily isolated and sequenced using conventional procedures (e.g., by using oligonucleotide probes that are capable of binding specifically to genes encoding the heavy and light chains of murine antibodies). Hybridoma cells as described above or IL-4Rα-specific phage clones of the application can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the application, or can be substituted for the variable domains of one antigen-combining site of an antibody of the application to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism.

Human and Humanized Antibodies

The anti-IL-4Rα antibodies (e.g., full-length anti-IL-4Rα antibodies) can be humanized antibodies or human antibodies. Humanized forms of non-human (e.g., murine) antibody moieties are chimeric immunoglobulins, immunoglobulin chains, or fragments thereof (such as Fv, Fab, Fab′, F(ab′)2, scFv, or other antigen-binding subsequences of antibodies) that typically contain minimal sequence derived from non-human immunoglobulin. Humanized antibody moieties include human immunoglobulins, immunoglobulin chains, or fragments thereof (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibody moieties can also comprise residues that are found neither in the recipient antibody nor in the imported CDR or framework sequences. In general, the humanized antibody can comprise substantially at least one, and typically two, variable domains, in which all or substantially all of the CDR regions correspond to those of a non-human immunoglobulin, and all or substantially all of the FR regions are those of a human immunoglobulin consensus sequence.

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibody moieties are antibody moieties (U.S. Pat. No. 4,816,567), wherein substantially less than an intact human variable domain has been substituted by the corresponding sequence from a non-human species. In practice, humanized antibody moieties are typically human antibody moieties in which some CDR residues and possibly some FR residues are substituted by residues from analogous sites in rodent antibodies.

As an alternative to humanization, human antibody moieties can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

Anti-IL-4Rα Antibody Variants

In some embodiments, amino acid sequences of the anti-IL-4Rα antibody variants (e.g., full-length anti-IL-4Rα antibody) provided herein are contemplated. For example, it may be desirable to improve the binding affinity and/or other biological properties of the antibody. Amino acid sequences of an antibody variant may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding activity.

In some embodiments, anti-IL-4Rα antibody variants having one or more amino acid substitutions are provided. Sites of interest for substitutional mutagenesis include the HVRs and FRs. Amino acid substitutions may be introduced into an antibody of interest and the products screened for a desired activity, e.g., improved bioactivity, retained/improved antigen binding activity, decreased immunogenicity, or improved ADCC or CDC.

Conservative substitutions are shown in Table 6 below.

TABLE 6
CONSERVATIVE SUBSTITUTIONS

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a. hydrophobic: Norleucine, Met, Ala, Val, Leu, Ile;
b. neutral hydrophilic: Cys, Ser, Thr, Asn, Gln;
c. acidic: Asp, Glu;
d. basic: His, Lys, Arg;
e. residues that influence chain orientation: Gly, Pro;
f. aromatic: Trp, Tyr, Phe.

Non-conservative substitutions will entail exchanging a member of one of these classes for another class.

An exemplary substitutional variant is an affinity matured antibody, which may be conveniently generated, e.g., using phage display-based affinity maturation techniques. Briefly, one or more CDR residues are mutated and the variant antibody moieties displayed on phage and screened for a particular biological activity (e.g., bioactivity based on TF-1 cell proliferation assay or binding affinity). Alterations (e.g., substitutions) may be made in HVRs, e.g., to improve bioactivity based on TF-1 cell proliferation assay or antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} and V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)).

In some embodiments of affinity maturation, diversity is introduced into the variable genes chosen for maturation by any of a variety of methods (e.g., error-prone PCR, chain shuffling, or oligonucleotide-directed mutagenesis). A secondary library is then created.

The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., Ala or Glu) to determine whether the interaction of the antibody with antigen is affected. Further substitutions may be introduced at the amino acid locations to demonstrate functional sensitivity to the initial substitutions. Alternatively, or additionally, a crystal structure of an antigen-antibody complex can be determined to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intra sequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

Fc Region Variants

In some embodiments, one or more amino acid modifications may be introduced into the Fc region of an antibody (e.g., a full-length anti-IL-4Rα antibody or anti-IL-4Rα Fc fusion protein) provided herein, thereby generating an Fc region variant. In some embodiments, the Fc region variant has enhanced ADCC effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-IL-4Rα antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the application contemplates an anti-IL-4Rα antibody variant (such as a full-length anti-IL-4Rα antibody variant) comprising an Fc region that possesses some but not all effector functions, which makes it a desirable candidate for applications in which the half-life of the anti-IL-4Rα antibody in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, there is provided an anti-IL-4Rα antibody (such as a full-length anti-IL-4Rα antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-IL-4Rα antibody (e.g., full-length anti-IL-4Rα antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-IL-4Rα antibody (such as a full-length anti-IL-4Rα antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-IL-4Rα antibodies (such as full-length anti-IL-4Rα antibodies) comprising any of the Fc variants described herein, or combinations thereof, are contemplated.

Glycosylation Variants

In some embodiments, an anti-IL-4Rα antibody (such as a full-length anti-IL-4Rα antibody) provided herein is altered to increase or decrease the extent to which the anti-IL-4Rα antibody is glycosylated. Addition or deletion of glycosylation sites to an anti-IL-4Rα antibody may be conveniently accomplished by altering the amino acid sequence of the anti-IL-4Rα antibody or polypeptide portion thereof such that one or more glycosylation sites are created or removed.

Wherein the anti-IL-4Rα antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-IL-4Rα antibody of the application may be made in order to create anti-IL-4Rα antibody variants with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding activity can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-IL-4Rα antibody (such as a full-length anti-IL-4Rα antibody) variants are provided comprising an Fc region wherein a carbohydrate structure attached to the Fc region has reduced fucose or lacks fucose, which may improve ADCC function. Specifically, anti-IL-4Rα antibodies are contemplated herein that have reduced fucose relative to the amount of fucose on the same anti-IL-4Rα antibody produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-IL-4Rα antibody is one wherein less than about 50%, 40%, 30%, 20%, 10%, or 5% of the N-linked glycans thereon comprise fucose. For example, the amount of fucose in such an anti-IL-4Rα antibody may be from 1% to 80%, from 1% to 65%, from 5% to 65% or from 20% to 40%. In some embodiments, the anti-IL-4Rα antibody is one wherein none of the N-linked glycans thereon comprise fucose, i.e., wherein the anti-IL-4Rα antibody is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-IL-4Rα antibody (such as a full-length anti-IL-4Rα antibody) variants are further provided with bisected oligosaccharides, e.g., in which a biantennary oligosaccharide attached to the Fc region of the anti-IL-4Rα antibody is bisected by GlcNAc. Such anti-IL-4Rα antibody (such as a full-length anti-IL-4Rα antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-IL-4Rα antibody (such as full-length anti-IL-4Rα antibody) variants with at least one galactose residue in the oligosaccharide attached to the Fc region are also provided. Such anti-IL-4Rα antibody variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-IL-4Rα antibody (such as a full-length anti-IL-4Rα antibody) variants comprising an Fc region are capable of binding to an FcγRIII. In some embodiments, the anti-IL-4Rα antibody (such as a full-length anti-IL-4Rα antibody) variants comprising an Fc region have ADCC activity in the presence of human effector cells (e.g., T cell) or have increased ADCC activity in the presence of human effector cells compared to the otherwise same anti-IL-4Rα antibody (such as a full-length anti-IL-4Rα antibody) comprising a human wild-type IgG1Fc region.

Cysteine Engineered Variants

In some embodiments, it may be desirable to create cysteine engineered anti-IL-4Rα antibodies (such as a full-length anti-IL-4Rα antibody) in which one or more amino acid residues are substituted with cysteine residues. In some embodiments, the substituted residues occur at accessible sites of the anti-IL-4Rα antibody. By substituting those residues with cysteine, reactive thiol groups are thereby positioned at accessible sites of the anti-IL-4Rα antibody and may be used to conjugate the anti-IL-4Rα antibody to other moieties, such as drug moieties or linker-drug moieties, to create an anti-IL-4Rα immunoconjugate, as described further herein. Cysteine engineered anti-IL-4Rα antibodies (e.g., full-length anti-IL-4Rα antibodies) may be generated as described, e.g., in U.S. Pat. No. 7,521,541.

Derivatives

In some embodiments, an anti-IL-4Rα antibody (such as a full-length anti-IL-4Rα antibody) provided herein may be further modified to contain additional non-proteinaceous moieties that are known in the art and readily available. The moieties suitable for derivatization of the anti-IL-4Rα antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the anti-IL-4Rα antibody may vary, and if more than one polymer are attached, they can be the same or different molecules. In general, the number and/or type of polymers used for derivatization can be determined based on considerations including, but not limited to, the particular properties or functions of anti-IL-4Rα antibody to be improved, whether the anti-IL-4Rα antibody derivative will be used in a therapy under defined conditions, etc.

Pharmaceutical Compositions

Also provided herein are compositions (such as pharmaceutical compositions, also referred to herein as formulations) comprising any of the anti-IL-4Rα antibodies (such as a full-length anti-IL-4Rα antibody), nucleic acids encoding the antibodies, vectors comprising the nucleic acids encoding the antibodies, or host cells comprising the nucleic acids or vectors described herein. In some embodiments, there is provided a pharmaceutical composition comprising any one of the anti-IL-4Rα antibodies described herein and a pharmaceutically acceptable carrier.

Suitable formulations of the anti-IL-4Rα antibodies are obtained by mixing an anti-IL-4Rα antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-IL-4Rα antibodies of this application into cells.

The formulation herein may also contain one or more active compounds in addition to the anti-IL-4Rα antibody (such as a full-length anti-IL-4Rα antibody) as necessary for the particular indication being treated, preferably those with complementary activities that do not adversely affect each other. For example, it may be desirable to further provide another IL-4R antagonist, an IL-1 antagonist, an IL-6 antagonist, an IL-6R antagonist, a TNF antagonist, an NASID, or combinations thereof in addition to the anti-IL-4Rα antibody. Such molecules are suitably present in combination in amounts that are effective for the purpose intended. The effective amount of such other agents depends on the amount of anti-IL-4Rα antibody present in the formulation, the type of disease or disorder or treatment, and other factors discussed above. These are generally used in the same dosages and with administration routes as described herein or about from 1% to 99% of the heretofore employed dosages.

The anti-IL-4Rα antibodies (e.g., full-length anti-IL-4Rα antibodies) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Sustained-release preparations may be prepared.

Sustained-release preparations of the anti-IL-4Rα antibodies (e.g., full-length anti-IL-4Rα antibodies) can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT′ (injectable microspheres composed of lactic acid-glycolic acid copolymer and leuprolide acetate), and poly-D (−)-3-hydroxybutyric acid. While polymers such as ethylene-vinyl acetate and lactic acid-glycolic acid enable release of molecules for over 100 days, certain hydro gels release proteins for shorter time periods. When encapsulated antibody remain in the body for a long time, they can denature or aggregate as a result of exposure to moisture at 37° C., resulting in a loss of biological activity and possible changes in immunogenicity. Rational strategies can be devised for stabilization of anti-IL-4Rα antibodies depending on the mechanism involved. For example, if the aggregation mechanism is discovered to be intermolecular S-S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-IL-4Rα antibody (such as a full-length anti-IL-4Rα antibody) is formulated in a buffer comprising a citrate, NaCl, acetate, succinate, glycine, polysorbate 80 (Tween 80), or any combination of the foregoing.

The formulations to be used for in vivo administration should be sterile. This is readily accomplished by, e.g., filtration through sterile filtration membranes.

Methods of Treatment Using Anti-IL-4Rα Antibodies

The anti-IL-4Rα antibodies (e.g., full-length anti-IL-4Rα antibodies) and/or compositions of the application can be administered to individuals (e.g., mammals such as humans) to treat a disease and/or disorder with deregulated IL-4 and/or IL-4Rα function, or characterized by high IL-4 and/or IL-4Rα expression and/or abnormal IL-4/IL-4Rα function, for example, asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis. The present application thus in some embodiments provides a method of treating a disease and/or disorder associated with high expression levels of IL-4 and/or IL-4Rα, and disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) in an individual comprising administering to the individual an effective amount of a composition (such as a pharmaceutical composition) comprising an anti-IL-4Rα antibody (e.g., a full-length anti-IL-4Rα antibody), such as any one of the anti-IL-4Rα antibodies (e.g., full-length anti-IL-4Rα antibodies) described herein. In some embodiments, the individual is human.

For example, in some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody (e.g., full-length anti-IL-4Rα antibody) comprising a heavy chain variable domain (V_{H}) comprising an HC-CDR1 comprising SYAMH (SEQ ID NO: 1); an HC-CDR2 comprising GISX₁X₂X₃X₄STYYANSVKG (SEQ ID NO: 78), wherein X₁ is P, S, H, G, or Y, X₂ is S, T, or N, X₃ is G or S, X₄ is 5, V, G, T, A, or N; and an HC-CDR3 comprising X₁X₂X₃X₄YRGGMDV (SEQ ID NO: 79), wherein X₁ is V or S, X₂ is K, F, or R, X₃ is P, V, G, R, S, or L, X₄ is G, A, R, K, or L; and a V_{L} comprising an LC-CDR1 comprising RASQX₁X₂SX₃AYLA (SEQ ID NO: 80), wherein X₁ is G, S, N, or D, X₂ is I, V, or A, X₃ is S, T, or N; an LC-CDR2 comprising GTSRRAT (SEQ ID NO: 41); and an LC-CDR3 comprising QLYGX₁X₂SVT (SEQ ID NO: 81), wherein X₁ is A, S, T, or R, X₂ is T or S. In some embodiments, the anti-IL-4Rα antibody is a full-length antibody. In some embodiments, the full-length anti-IL-4Rα antibody is an IgG1 or IgG4 antibody. In some embodiments, the individual is human.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of any one of SEQ ID NOs: 2-16, and an HC-CDR3 comprising the amino acid sequence of any one of SEQ ID NOs: 17-30, or a variant thereof comprising up to 5 amino acid substitutions in the HC-CDRs; and a V_{L} comprising an LC-CDR1 comprising the amino acid sequence of any one of SEQ ID NOs: 31-40, an LC-CDR2 comprising the amino acid sequence of SEQ ID NO: 41, and an LC-CDR3 comprising the amino acid sequence of any one of SEQ ID NOs: 42-47, or a variant thereof comprising up to 5 amino acid substitutions in the LC-CDRs.

In some embodiments, there is provided a method of treating a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising a V_{H} comprising the amino acid sequence of any one of SEQ ID NOs: 48-64, or a variant thereof having at least about 90% sequence identity to the amino acid sequence of any one of SEQ ID NOs: 48-64, and a V_{L} comprising the amino acid sequence of any one of SEQ ID NOs: 65-77, or a variant thereof having at least about 90% sequence identity to the amino acid sequence of any one of SEQ ID NOs: 65-77.

In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 48 and a V_{L} comprising the amino acid sequence of SEQ ID NO: 65. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18, or a variant thereof comprising up to 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 49 and a V_{L} comprising the amino acid sequence of SEQ ID NO: 66. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 50 and a V_{L} comprising the amino acid sequence of SEQ ID NO: 66. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 51 and a V_{L} comprising the amino acid sequence of SEQ ID NO: 67. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 52 and a V_{L} comprising the amino acid sequence of SEQ ID NO: 67. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22, or a variant thereof comprising up to 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 53 and a V_{L} comprising the amino acid sequence of SEQ ID NO: 66. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23, or a variant thereof comprising up to 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 54 and a V_{L} comprising the amino acid sequence of SEQ ID NO: 67. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24, or a variant thereof comprising up to 5 amino acid substitutions in the HC-CDRs; and a V_{L} comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 55 and a V_{L} comprising the amino acid sequence of SEQ ID NO: 68. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:10, and an HC-CDR3 comprising the amino acid sequence of SEQ ID NO: 25, or a variant thereof comprising up to 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 56 and a V_{L} comprising the amino acid sequence of SEQ ID NO: 69. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:11, and an HC-CDR3 comprising the amino acid sequence of SEQ ID NO: 26, or a variant thereof comprising up to 5 amino acid substitutions in the HC-CDRs; and a V_{L} comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 57 and a V_{L} comprising the amino acid sequence of SEQ ID NO: 70. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:4, and an HC-CDR3 comprising the amino acid sequence of SEQ ID NO: 19, or a variant thereof comprising up to 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 50 and a V_{L} comprising the amino acid sequence of SEQ ID NO: 71. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:2, and an HC-CDR3 comprising the amino acid sequence of SEQ ID NO: 27, or a variant thereof comprising up to 5 amino acid substitutions in the HC-CDRs; and a V_{L} comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 58 and a V_{L} comprising the amino acid sequence of SEQ ID NO: 72. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:12, and an HC-CDR3 comprising the amino acid sequence of SEQ ID NO: 28, or a variant thereof comprising up to 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 59 and a V_{L} comprising the amino acid sequence of SEQ ID NO: 73. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:13, and an HC-CDR3 comprising the amino acid sequence of SEQ ID NO: 28, or a variant thereof comprising up to 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 60 and a V_{L} comprising the amino acid sequence of SEQ ID NO: 74. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:14, and an HC-CDR3 comprising the amino acid sequence of SEQ ID NO: 29, or a variant thereof comprising up to 5 amino acid substitutions in the HC-CDRs; and a V_{L} comprising an LC-CDR1 comprising the amino acid sequence of SEQ ID NO: 39, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 61 and a V_{L} comprising the amino acid sequence of SEQ ID NO: 75. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:15, and an HC-CDR3 comprising the amino acid sequence of SEQ ID NO: 20, or a variant thereof comprising up to 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 62 and a V_{L} comprising the amino acid sequence of SEQ ID NO: 69. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:7, and an HC-CDR3 comprising the amino acid sequence of SEQ ID NO: 30, or a variant thereof comprising up to 5 amino acid substitutions in the HC-CDRs; and a V_{L} comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 63 and a V_{L} comprising the amino acid sequence of SEQ ID NO: 76. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4.

In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, there is provided a method of treating an individual having a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis) comprising administering to the individual an effective amount of a pharmaceutical composition comprising an anti-IL-4Rα antibody comprising: a V_{H} comprising an HC-CDR1 comprising the amino acid sequence of SEQ ID NO: 1, an HC-CDR2 comprising the amino acid sequence of SEQ ID NO:16, and an HC-CDR3 comprising the amino acid sequence of SEQ ID NO: 20, or a variant thereof comprising up to 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47, or a variant thereof comprising up to 5 amino acid substitutions in the LC-CDRs.

In some embodiments, the anti-IL-4Rα antibody provided herein comprises a V_{H} comprising the amino acid sequence of SEQ ID NO: 64 and a V_{L} comprising the amino acid sequence of SEQ ID NO: 77. In some embodiments, the anti-IL-4Rα antibody provided herein is a full-length anti-IL-4Rα antibody comprising IgG1 or IgG4 constant domains. In some embodiments, the IgG1 is human IgG1. In some embodiments, the IgG4 is human IgG4. In some embodiments, the heavy chain constant region comprises or consists of the amino acid sequence of SEQ ID NO: 84. In some embodiments, the heavy chain constant region comprises or consists of the amino acid sequence of SEQ ID NO: 85. In some embodiments, the light chain constant region comprises or consists of the amino acid sequence of SEQ ID NO: 86.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any of 70, 80, 90, or 100 years old). In some embodiments, the individual is diagnosed with or genetically prone to one or more of the diseases or disorders described herein (such as asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, or nephrosis). In some embodiments, the individual has one or more risk factors associated with one or more diseases or disorders described herein.

The present application in some embodiments provides a method of delivering an anti-IL-4Rα antibody (such as any one of the anti-IL-4Rα antibodies described herein, e.g., an isolated anti-IL-4Rα antibody) to a cell expressing IL-4Rα on its surface in an individual, the method comprising administering to the individual a composition comprising the anti-IL-4Rα antibody.

Many diagnostic methods for disease exhibiting abnormal IL-4 and/or IL-4Rα expression and the clinical delineation of those diseases are known in the art. Such methods include, but are not limited to, e.g., immunohistochemistry, PCR, and fluorescent in situ hybridization (FISH).

In some embodiments, the anti-IL-4Rα antibodies (e.g., full-length anti-IL-4Rα antibodies) and/or compositions of the application are administered in combination with a second, third, or fourth agent (including, e.g., another IL-4R antagonist, an IL-1 antagonist, an IL-6 antagonist, an IL-6R antagonist, a TNF antagonist, an NASID, or combinations thereof), to treat diseases or disorders involving abnormal IL-4/IL-4Rα expression.

Dosing and Method of Administering the Anti-IL-4Rα Antibodies

The dose of the anti-IL-4Rα antibody (such as isolated anti-IL-4Rα antibody) compositions administered to an individual (such as a human) may vary with the particular composition, the mode of administration, and the type of disease being treated. Depending on the severity of the condition, the frequency and the duration of the treatment can be adjusted. In some embodiments, the amount of the composition (such as composition comprising isolated anti-IL-4Rα antibody) is effective to produce an objective response (such as a partial response or a complete response) in the treatment of a disease. In some embodiments, the amount of the anti-IL-4Rα antibody composition is sufficient to produce a complete response in the individual. In some embodiments, the amount of the anti-IL-4Rα antibody composition is sufficient to produce a partial response in the individual. In some embodiments, the amount of the anti-IL-4Rα antibody composition administered (for example when administered alone) is sufficient to produce an overall response rate of more than about any of 20%, 25%, 30%, 35%, 40%, 45%, 50%, 55%, 60%, 64%, 65%, 70%, 75%, 80%, 85%, or 90% among a population of individuals treated with the anti-IL-4Rα antibody composition. Responses of an individual to the treatment of the methods described herein can be determined.

In some embodiments, the amount of the composition (such as composition comprising isolated anti-IL-4Rα antibody) is sufficient to prolong progress-free survival of the individual. In some embodiments, the amount of the composition is sufficient to prolong overall survival of the individual. In some embodiments, the amount of the composition (for example when administered along) is sufficient to produce clinical benefit of more than about any of 50%, 60%, 70%, or 77% among a population of individuals treated with the anti-IL-4Rα antibody composition.

In some embodiments, the amount of the composition (such as composition comprising isolated anti-IL-4Rα antibody), alone or in combination with a second, third, and/or fourth agent, is an amount, for example, sufficient to decrease the incidence of asthma exacerbations; improve one or more asthma associated parameters; and/or improve one or more symptoms or indicia of an upper airway inflammatory condition by at least about any of 10%, 20%, 30%, 40%, 50%, 60%, 70%, 80%, 90%, 95% or 100% compared with the same subject prior to treatment or compared with the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-IL-4Rα antibody (such as a full-length anti-IL-4Rα antibody) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regimen. In some embodiments, the amount of the composition is more than about 80%, 90%, 95%, or 98% of the MTD.

In some embodiments, the amount of an anti-IL-4Rα antibody (such as a full-length anti-IL-4Rα antibody) in the composition is included in a range of about 0.001 μg to about 1000 μg.

In some embodiments of any of the above aspects, the effective amount of anti-IL-4Rα antibody (such as a full-length anti-IL-4Rα antibody) in the composition is in the range of about 0.1 μg/kg to about 100 mg/kg of total body weight.

The anti-IL-4Rα antibody compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, or transdermal. In some embodiments, sustained continuous release formulation of the composition may be used. In some embodiments, the composition is administered intravenously. In some embodiments, the composition is administered intraportally. In some embodiments, the composition is administered intraarterially. In some embodiments, the composition is administered intraperitoneally. In some embodiments, the composition is administered intrahepatically. In some embodiments, the composition is administered by hepatic arterial infusion. In some embodiments, the administration is to an injection site distal to a first disease site.

Articles of Manufacture and Kits

In some embodiments of the application, there is provided an article of manufacture containing materials useful for the treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis), or for delivering an anti-IL-4Rα antibody (such as a full-length anti-IL-4Rα antibody) to a cell expressing IL-4Rα on its surface. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for treating a disease or disorder described herein, and may have a sterile access port (for example the container may be an intravenous solution bag or a vial having a stopper pierceable by a hypodermic injection needle). At least one active agent in the composition is an anti-IL-4Rα antibody of the application. The label or package insert indicates that the composition is used for treating the particular condition. The label or package insert will further comprise instructions for administering the anti-IL-4Rα antibody composition to the patient. Articles of manufacture and kits comprising combinatorial therapies described herein are also contemplated.

Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a disease and/or disorder with deregulated IL-4 and/or IL-4Rα function, or characterized by high IL-4 and/or IL-4Rα expression and/or abnormal IL-4/IL-4Rα function (such as asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis).

Additionally, the article of manufacture may further comprise a second container comprising a pharmaceutically-acceptable buffer, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

Kits are also provided that are useful for various purposes, e.g., for treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis), or for delivering an anti-IL-4Rα antibody (such as a full-length anti-IL-4Rα antibody) to a cell expressing IL-4Rα on its surface, optionally in combination with the articles of manufacture. Kits of the application include one or more containers comprising anti-IL-4Rα antibody composition (or unit dosage form and/or article of manufacture), and in some embodiments, further comprise other agents (such as the agents described herein) and/or instructions for use in accordance with any of the methods described herein. The kit may further comprise a description of selection of individuals suitable for treatment. Instructions supplied in the kits of the application are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-IL-4Rα antibody (such as a full-length anti-IL-4Rα antibody). In some embodiments, the kit comprises a) a composition comprising any one of the anti-IL-4Rα antibodies described herein, and b) an effective amount of at least one other agent, wherein the other agent enhances the effect (e.g., treatment effect, detecting effect) of the anti-IL-4Rα antibody. In some embodiments, the kit comprises a) a composition comprising any one of the anti-IL-4Rα antibodies described herein, and b) instructions for administering the anti-IL-4Rα antibody composition to an individual for treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis). In some embodiments, the kit comprises a) a composition comprising any one of the anti-IL-4Rα antibodies described herein, b) an effective amount of at least one other agent, wherein the other agent enhances the effect (e.g., treatment effect, detecting effect) of the anti-IL-4Rα antibody, and c) instructions for administering the anti-IL-4Rα antibody composition and the other agent(s) to an individual for treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis). The anti-IL-4Rα antibody and the other agent(s) can be present in separate containers or in a single container. For example, the kit may comprise one distinct composition or two or more compositions wherein one composition comprises an anti-IL-4Rα antibody and another composition comprises another agent.

In some embodiments, the kit comprises a nucleic acid (or a set of nucleic acids) encoding an anti-IL-4Rα antibody (such as a full-length anti-IL-4Rα antibody). In some embodiments, the kit comprises a) a nucleic acid (or a set of nucleic acids) encoding an anti-IL-4Rα antibody, and b) a host cell for expressing the nucleic acid (or a set of nucleic acids). In some embodiments, the kit comprises a) a nucleic acid (or a set of nucleic acids) encoding an anti-IL-4Rα antibody, and b) instructions for i) expressing the anti-IL-4Rα antibody in a host cell, ii) preparing a composition comprising the anti-IL-4Rα antibody, and iii) administering the composition comprising the anti-IL-4Rα antibody to an individual for the treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis). In some embodiments, the kit comprises a) a nucleic acid (or a set of nucleic acids) encoding an anti-IL-4Rα antibody, b) a host cell for expressing the nucleic acid (or a set of nucleic acids), and c) instructions for i) expressing the anti-IL-4Rα antibody in the host cell, ii) preparing a composition comprising the anti-IL-4Rα antibody, and iii) administering the composition comprising the anti-IL-4Rα antibody to an individual for the treatment of a disease and/or disorder with deregulated IL-4 and/or IL-4Rα function, or characterized by high IL-4 and/or IL-4Rα expression and/or abnormal IL-4/IL-4Rα function (e.g., asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis).

The kits of the application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the anti-IL-4Rα antibody compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-IL-4Rα antibody (such as a full-length anti-IL-4Rα antibody) as disclosed herein to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-IL-4Rα antibody and pharmaceutical compositions and instructions for use and packaged in quantities sufficient for storage and use in pharmacies, for example, hospital pharmacies and compounding pharmacies.

Those skilled in the art will recognize that several embodiments are possible within the scope and spirit of this application. The application will now be described in greater detail by reference to the following non-limiting examples. The following examples further illustrate the application but, of course, should not be construed as in any way limiting its scope.

### EXAMPLES

In the experimental disclosure which follows, the following abbreviations apply: IL-4 (Interleukin-4); IL-13 (Interleukin-13); hIL-4 (human Interleukin-4); hIL-13 (human Interleukin-13); IL-4Rα (Interleukin-4 receptor α); Bavi-IL-4Rα (Biotin-Avi-IL-4Rα)

### Example 1: Generation of Recombinant Human IL-4Rα and Selection of Anti-IL-4Rα scFv Antibodies

Generation of Recombinant Human IL-4Rα

The coding sequence of the extracellular domain of human IL-4Rα (SEQ ID NO: 82) was synthesized and subcloned into the expression vector pTT5 using restriction enzyme recognition sites. His-tag or other conventionally used tags were used to tag IL-4Rα. “his or His” stands for His-tag, and “Avi” stands for Avi-tag.

The expression and purification of recombinant human IL-4Rα were carried out according to the manufacturer's protocol. Briefly, HEK-293F cells were transfected with the expression vectors using PEI, and the cells were cultured at 37° C., under 5% CO₂ and 120 rpm for 5 days. The culture media was collected and IL-4Rα proteins were purified using Ni Sepharose purification according to the manufacturer's protocol. Specifically, the Qiagen Ni-NTA superflow cartridges were used for immobilized metal affinity chromatography (IMAC) analysis. The cartridges were first equilibrated with buffer A1 (50 mM Na₃PO₄, 0.15M NaCl, pH 7.2) with a flow rate of 150 cm/h. The supernatant of the culture media, whose pH was adjusted to 7.2, flowed through the cartridges at room temperature with a flow rate of 150 cm/h. Next, buffer A1 (6 times the volume of that of the cartridges) was used to equilibrate the cartridges at 150 cm/h. A 50 mM PB solution (0.15M NaCl and 0.2M Imidazole, pH 7.2) with a volume that is 10 times that of the cartridges was used to wash the cartridges and the elution containing IL-4Rα was collected.

Generation of Biotinylated IL-4Rα Antigen

Biotinylation of Avi-IL-4Rα using the biotin ligase B0101A (GeneCopoeia) was carried out according to the manufacturer's protocol. Briefly, buffer AB and BirA ligase were added to Avi-IL-4Rα, followed by 2 hours of incubation at 30° C. The biotinylated IL-4Rα is referred to as Bavi-IL-4Rα. The efficiency of biotinylation was measured using ELISA. Briefly, Bavi-IL-4Rα was serially diluted at a 1:2 ratio, from a starting concentration of 500 ng/mL, before being used to coat the ELISA plate. SA-HRP was used for detection and standard biotinylation products were used as a control. The biotinylation efficiency was determined to be 70%.

Selection of Anti-IL-4Rα scFv Antibodies

Generation of yeast scFv antibody display library: RNA collected from 2000 human blood samples was reverse-transcribed into cDNA, and the V_{H} and V_{K} fragments were amplified using V_{H}- and V_{K}-specific primers. Upon gel extraction and purification, scFvs were generated by linking V_{H} and V_{K}, and were cloned into the yeast display plasmid PYD1, which were then electroporated into yeast to generate the yeast scFv antibody display library.

Selection of anti-IL-4Rα scFv antibodies: scFvs which recognized IL-4Rα were enriched and selected from the yeast display library. Briefly, magnetic-activated cell sorting (MACS) was used to enrich cells expressing anti-IL-4Rα scFv antibodies. 1000 OD yeast cells were subjected to centrifugation for 5 minutes at 2500 g. Cell pellets were obtained and resuspended in 1 L of SGCAA culture media with OD600=1 as the starting concentration. Expression was induced for 40-48 hours at 20° C. and 250 rpm. After centrifugation and washing with PBSM, the pellet was resuspended in 5-10 times volume of 1 μM Bavi-IL-4Rα (in PBSM), and incubated for 1 hour at 4° C. After centrifugation and washed with PBSM, unbound antigens were washed off. Magnetic beads were added and mixed thoroughly before incubation for 30 minutes at 4° C. on a rotator. The supernatant was discarded after centrifugation at 2500 g for 5 minutes, and the pellet was resuspended in PBSM with 5-10 times the volume. Cells were added to the selection column until all cells were passed through the column. Bound cells were collected and upon further culturing and centrifugation were subjected to plasmid isolation.

Generation of phage display library and selection of scFv antibodies: scFv antibody fragments from the selected yeast cells were PCR amplified using scFv-F and scFv-R primers. To generate phage display libraries, the scFv fragments were then cloned into the phage display vector pDAN5. Upon ligation, the vector was used to transduce TG1 phage display electroporation-competent cells to obtain the phage scFv antibody display library. scFv antibodies specific to IL-4Rα were isolated from the phage display library in a series of repeated selection cycles. Briefly, phage scFv library (2×10¹¹ PFU) was added to biotinylated IL-4Rα, and incubated for 2 hours at 37° C. IL-4Rα with phage bound was captured on streptavidin coated magnetic beads. Unbound phages were washed away. After washing with TBST 8-15 times (increasing number of washes for every round of selection), phages that specifically bound to IL-4Rα were washed off with Glycine-HCl (pH2.2). These phages were used to transduce TG1 cells in the log phase, with the addition of Ampicillin, and cultured for 1 hour. Upon the addition of helper phage, the cells were cultured on a rocking bed overnight at 200 rpm at 28° C. Culture media was collected the next day, centrifuged to obtain the supernatant, and was subjected to the next round of selection. A panel of positive scFv antibodies were obtained at the end of the selection process.

Binding ELISA: Monoclonal scFv antibodies were selected and subjected to ligand binding assay. The binding ELISA assay was designed to identify scFv antibodies that bound human IL-4Rα. Briefly, a 96-well plate was coated with human IL-4Rα in PBS at 0.4 g/well and left overnight at 4° C. Before loading the scFv antibodies, the plates were washed with TBST, blocked for 1-2 hours at 37° C. using 5% milk and washed again with TBST. Each scFv sample was first diluted to 40 μg/mL, and 150 μL of the scFv sample was added to the first row of wells. The 40 μg/mL scFv samples were then serially diluted at a 1:3 ratio and added to the remaining wells. After incubating for 1 hour at 37° C., followed by washing with TBST 6 times, 100 μl of the primary antibody and secondary antibody mixture (mouse anti-flag (1:2500) and anti-mouse FC-AP (1:2000)) was added to each well. After incubation for 1 hour under 37° C., the plate was washed 3 times using TBST. pNPP was then added at 50 μL/well and incubated for 10-20 minutes at 37° C. 3M NaOH was used to stop the reaction. The ELISA results (OD405) were then analyzed and the binding curves were generated by GraphPad Prism 5 software (GraphPad Software).

Neutralization of Biological Effect of hIL-4 In Vitro

The IL-4-mediated signal transduction pathway has been documented extensively in the literature (for example, see review of Hebenstreit et al. 2006 Cytokine Growth Factor Rev. 17(3):173-88, 2006). IL-4 can stimulate two types of receptor complexes, type I and type II. Type I receptor complexes are formed by the binding of IL-4 to IL-4R and the subsequent heterodimerization with the common gamma chain. Alternatively, IL4/IL4R complex can heterodimerize with the IL-13 receptor 1 to form type II receptor complexes. Both type I and type II complexes signal mainly through STAT6. Therefore, the ability of the selected antibodies to block signaling through STAT6 was assessed as described below.

A bioassay was developed to determine the purified anti-IL-4Rα antibodies to neutralize hIL-4-mediated cellular function in vitro. A cell line HEK-Blue™ IL-4/IL-13 cells were purchased from InvivoGen, with high sensitivity to hIL-4 and hIL-13. HEK-Blue™ IL-4/IL-13 cells were generated by stable transfection of HEK293 cells with the human STAT6 gene to obtain a fully active STAT6 pathway, and the cells were further transfected with a STAT6-inducible SEAP reporter gene. HEK-Blue™ IL-4/IL-13 cells can produce SEAP in response to IL-4 or IL-13 stimulation. The levels of STAT6-induced SEAP secreted in the supernatant can be easily determined using QUANTI-Blue™ Solution. Stimulation of HEK-Blue™ IL-4/IL-13 cells with recombinant hIL-4 can be blocked by the anti-IL4Rα antibody. The inhibition assay was performed as follows: the cells were seeded onto 96-well plates at 6×10⁴ cells/well and incubated overnight at 37° C., 5% CO₂. The anti-IL-4R antibodies were serially diluted at a 1:4 ratio, from a starting concentration of 111 nM to 4.23E-04 nM, and 20 μL of each diluted anti-IL-4Rα antibodies were added to the cells, along with 20 μL of hIL-4 with the final concentration of 1.2 ng/ml. Cells were then incubated at 37° C., 5% CO₂ for 24 hrs. The SEAP activity was detected and quantified using QUANTI-Blue™ Solution according to the QUANTI-Blue™ Standard procedure (OD655 nm). The IC50 values were calculated using GraphPad Prism 5 software (GraphPad Software).

Neutralization of Biological Effect of Human IL-13 In Vitro:

Since IL-4Rα is also a modulator for IL-13 activity through its binding to IL-13/IL-13R complex, the selected antibodies were tested for their ability to block IL-13 activity in the HEK293 STAT6 SEAP assay described above, with the replacement of hIL-4 with hIL-13, and the final concentration of hIL-13 is 4 ng/ml.

### Example 2: Generation and Characterization of Full-Length Human Anti-IL-4Rα Antibodies

Generation of Full-Length Anti-IL-4Rα Antibodies

The most potent scFv antibodies were reformatted as human IgG1 or IgG4 antibody molecules with a human IgG1 or IgG4 heavy chain constant domain, and a human kappa light chain constant domain. V_{L} were amplified from the prokaryotic expression vector and introduced into eukaryotic expression vectors pTT5-L (containing kappa constant domain) and V_{H} were introduced into pTT5-H1 (containing IgG1 heavy chain constant domain), or pTT5-H4 (containing IgG4 heavy chain constant domain). Plasmids expressing the light or heavy chains were extracted and used to co-transfect 293F cells. After the cells were cultured at 37° C., 5% CO₂ and 120 rpm for 5 days, the culture media was purified using Protein A affinity chromatography. Briefly, Protein A column was first equilibrated with a PBS buffer containing 50 mM PBS and 0.15M NaCl (pH7.2), at a flow rate of 150 cm/h and with a volume that is six times the volume of the column. The supernatant of the culture media (pH was adjusted to 7.2) was passed through the column at the flow rate of 150 cm/h. Upon further equilibration, the column was washed using 50 mM sodium citrate (pH3.5) and the elution was collected. Out of the full-length antibodies that were generated, C27 was selected as the lead parent antibody. Using the scFv of C27, a phage scFv display library containing mutations in the CDR regions was generated. Variants that were able to bind human IL-4Rα with high affinity, and with low dissociation rate were assessed for biological activity in neutralizing of hIL-4 and hIL-13. The scFv antibodies that showed improved biological activity as compared to the scFv of C27 were used to generate full-length antibodies. A further round of selection of the full-length antibodies was carried out. The selected lead-optimized antibodies were then subjected to further biochemical and biological analysis.

Affinity of Anti-IL-4Rα Antibodies

The affinity of the parent antibody C27 and the lead-optimized antibodies (reformatted as human IgG4) for human IL-4Rα was evaluated using ELISA. All the lead-optimized antibodies exhibited good binding affinity to IL-4Rα(data not shown).

Characterization of Binding Affinity and Dissociation Constant (Kd)

The binding affinity of the anti-IL-4Rα antibodies (reformatted as human IgG4) to human IL-4Rα was characterized using Biacore T200 (GE). The full-length anti-IL-4Rα antibodies were stabilized on sensor chip CMS. The affinities for human IL-4Rα at various concentrations were measured. The range of concentrations included 12.5, 10, 5, 2.5, 1.25, and 0.625 nM. Using the SPR technology, the association and dissociation rates were measured, and binding affinity was determined. Kon, Koff, and Kd of the anti-IL-4Rα antibodies were shown in Table 7.

TABLE 7

| Antibody | Kon(1/Ms) | Koff(1/s) | Kd (M) |
|---|---|---|---|
| C27 | 7.190×E5 | 1.270×E−2 | 1.760×E−8 |
| C27-6-33 | 8.045×E4 | 1.704×E−4 | 2.118×E−9 |
| C27-7-33 | 1.335×E5 | 1.344×E−4 | 1.007×E−9 |
| C27-24-56 | 9.271×E4 | 1.729×E−4 | 1.865×E−9 |
| C27-47-56 | 1.070×E5 | 1.948×E−4 | 1.821×E−9 |
| C27-23-13 | 9.214×E4 | 3.085×E−4 | 3.348×E−9 |
| C27-33-33 | 1.194×E5 | 4.467×E−4 | 3.742×E−9 |
| C27-56-56 | 8.447×E4 | 2.013×E−4 | 2.383×E−9 |
| C27-36-36 | 1.369×E5 | 3.506×E−4 | 2.562×E−9 |
| C27-55-55 | 1.536×E5 | 3.787×E−4 | 2.466×E−9 |
| C27-Y2-Y2 | 1.393×E5 | 7.799×E−4 | 5.599×E−9 |
| C27-78-78 | 1.147×E5 | 4.156×E−4 | 3.623×E−9 |
| C27-82-58 | 1.517×E5 | 5.569×E−4 | 3.672×E−9 |

### Example 3: Inhibition of Human IL-4 and Human IL-4Rα Interaction

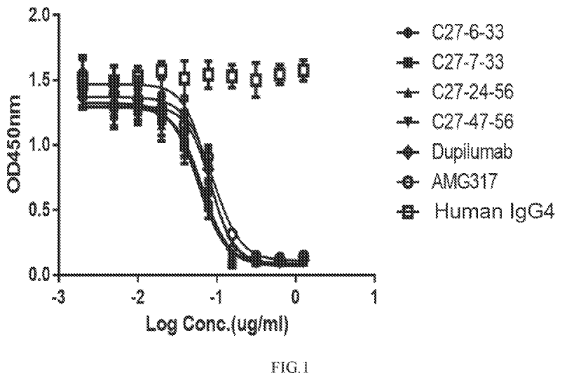

Competitive ELISA: The second assay was designed to identify the anti-IL-4Rα antibodies that were capable of inhibiting the binding of hIL-4 to hIL-4Rα, as measured by competitive ELISA. Briefly, a 96-well plate was coated with 100 μL, 2.5 μg/mL SA (Streptavidin) solution, and incubated overnight at 4° C., followed by washed with TBST. Biotinylated IL-4 with the final concentration of 2 μg/mL was added into the plate and incubated at 37° C. for 1 hour. Then the plate was washed with TBST. The anti-IL-4Rα antibody samples were first diluted to 10 μg/mL, and 1004, of the sample was added to the first row of wells. 10 μg/mL anti-IL-4Rα antibody samples were then serially diluted at a 1:2 ratio and added to each of the remaining rows of wells. 504 of 0.5 μg/mL IL-4R with mouse Fc-tag was added to each well. After incubating for 1 hour at 37° C., the wells were washed with TBST 6 times. 1004 of anti-mouse-Fc-HRP (1:10,000) was then added to each well and incubated for 1 hour at 37° C. The wells were washed with TBST 6 times before adding 1004/well of TMB, and incubated for 5-10 minutes at 37° C. 2M H₂SO₄ was used to stop the reaction. The ELISA results (OD450) were read, and the binding curves were generated by GraphPad Prism 5 software (GraphPad Software). Reference antibody Dupilumab (Regeneron, anti-IL-4Rα antibody) and AMG317 (Amgen, anti-IL-4Rα antibody) were used as positive controls, Human IgG4, kappa Isotype Control (Cat #HG4K, Sino Biological Inc.) was used as negative control, abbreviated as Human IgG4 in FIG. 1.

As shown in FIG. 1 and Table 8, the lead-optimized anti-IL4Rα antibodies (reformatted as human IgG4) were able to block human IL-4 from binding to human IL-4Rα, and showed better efficacy when compared to the reference antibody Dupilumab or AMG317.

TABLE 8

| Antibody | C27-6-33 | C27-7-33 | C27-24-56 | C27-47-56 | Dupilumab | AMG317 |
|---|---|---|---|---|---|---|
| IC50 | 0.07812 | 0.05824 | 0.06324 | 0.06508 | 0.08382 | 0.08906 |
| (μg/mL) | | | | | | |

### Example 4: Neutralization of Biological Effect of Human IL-4 In Vitro

The human IL-4 neutralization assay using HEK-Blue™ IL-4/IL-13 cells in vitro was performed as described in Example 1. Dupilumab was used as a control.

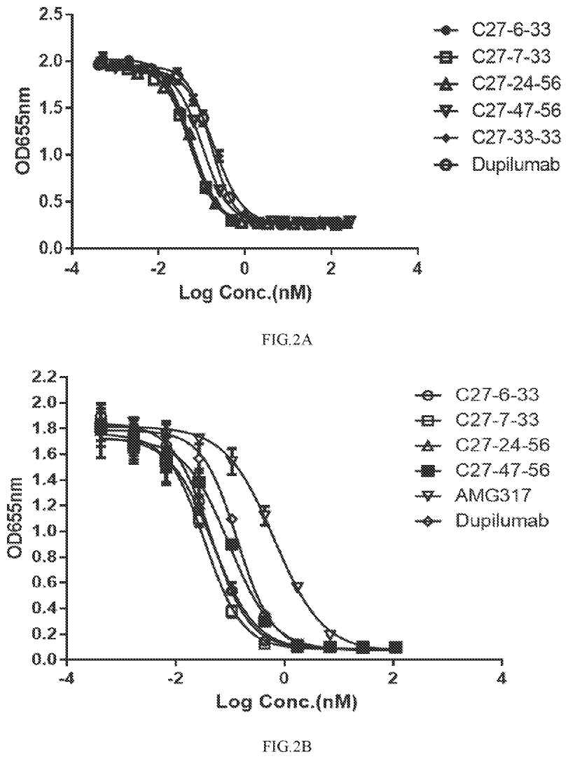

As shown in FIG. 2A and Table 9, all the lead optimized anti-IL-4Rα antibodies (reformatted as human IgG4) exhibited good efficacy in blocking human IL-4 signaling through the STATE pathway, and had better or comparable activity in neutralizing human IL-4 biological effect when compared to the reference antibody Dupilumab.

TABLE 9

| Antibody | IC50(nM) | Antibody | IC50(nM) |
|---|---|---|---|
| C27 | 17.9 | C27-55-55 | 0.08525 |
| C27-6-33 | 0.05368 | C27-Y2-Y2 | 0.1375 |
| C27-7-33 | 0.05405 | C27-82-58 | 0.2831 |
| C27-24-56 | 0.06316 | C27-58-58 | 0.4098 |
| C27-47-56 | 0.1032 | C27-56-56 | 0.1502 |
| C27-33-33 | 0.1949 | C27-54-54 | 0.3397 |
| C27-67-67 | 0.076 | C27-53-53 | 0.2926 |
| C27-78-78 | 0.1031 | C27-52-52 | 0.4757 |
| C27-36-36 | 0.1254 | Dupilumab | 0.1652 |

### Example 5: Neutralization of Biological Effect of Human IL-13 In Vitro

The human IL-13 neutralization assay using HEK-Blue™ IL-4/IL-13 cells in vitro was performed as described in Example 1. Dupilumab and AMG317 were used as controls.

As shown in FIG. 2B and Table 10, the optimized anti-IL-4Rα antibodies (reformatted as human IgG4) C27-6-33, C27-7-33, C27-24-56, and C27-47-56 exhibited better efficacy in blocking human IL-13 signaling through the STATE pathway, and neutralizing the biological effect of human IL-13 when compared to the reference antibody AMG317 or Dupilumab.

TABLE 10

| Antibody | C27-6-33 | C27-7-33 | C27-24-56 | C27-47-56 | AMG317 | Dupilumab |
|---|---|---|---|---|---|---|
| IC50(nM) | 0.04462 | 0.03435 | 0.0482 | 0.09661 | 0.6495 | 0.1363 |

### Example 6: Inhibition of TF-1 Cell Proliferation Assay

The ability of the anti-IL-4Rα antibodies to inhibit IL-4 or IL-13 dependent biological activity in vitro was assessed in a TF-1 proliferation assay. TF-1 is a human pre myeloid cell line established from a patient with erythroleukemia. This cell line is factor-dependent for survival and proliferation, for example, IL-4, IL-13, or GM-CSF. The bioactivity of anti-IL-4Rα antibodies in inhibiting IL-4 or IL-13 dependent TF-1 cell proliferation was tested. Dupilumab was used as a control. TF-1 cells were purchased from the American Type Culture Collection (ATCC® CRL-2003™). Briefly, prior to use, TF-1 cells were harvested in the logarithmic phase and washed 3 times with assay medium (the growth medium without IL-4 or IL-13). Cells were resuspended in the assay medium and added to a 96-well cell culture plate at 2×10⁴ cells/60 μL/well, and incubated for 1 hour in a humidified chamber at 37° C. and 5% CO₂. Then, anti-IL-4Rα antibodies were serially diluted at a 1:4 ratio, from a starting concentration of 55.6 nM to 2.12E-04 nM, 204, of the anti-IL-4Rα antibody serial dilutions were added to each well along with human IL-4 with the final concentration of 2 ng/mL or human IL-13 with the final concentration of 10 ng/mL, and the plates were incubated for 72 h at 37° C. and 5% CO₂. After 72 h, cell proliferation was analyzed using the CellTiter-Glo Luminescent Cell Viability Assay Kit (Promega) as per manufacturer's instructions. It is a homogeneous method to determine the number of viable cells in culture based on luminescent detection of ATP, which indicates the presence of metabolically active cells. The IC50 values were calculated using GraphPad Prism 5 software (GraphPad Software).

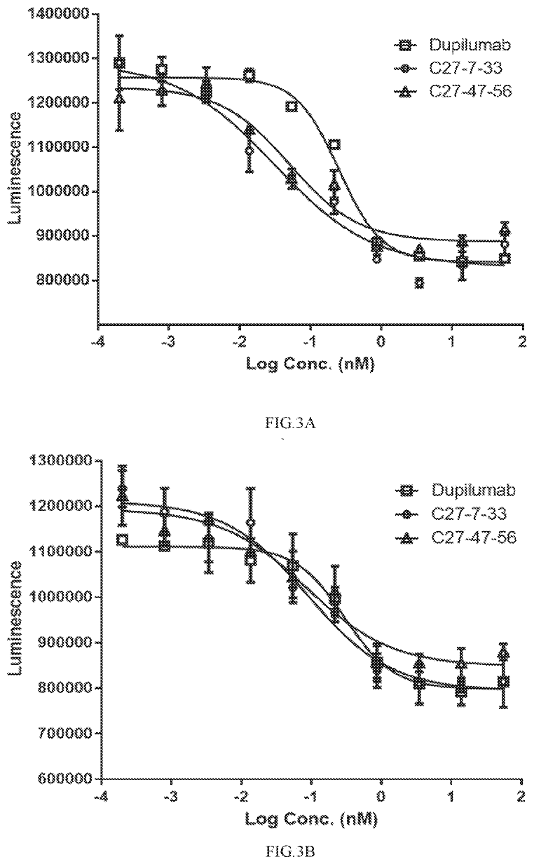

As shown in FIG. 3A and Table 11, the lead-optimized antibodies (reformatted as human IgG4) C27-7-33 and C27-47-56 were tested for their ability to inhibit IL-4 dependent TF-1 cell proliferation, the lead-optimized antibodies showed improved efficacy in inhibiting TF-1 cell proliferation when compared to the reference antibody Dupilumab.

TABLE 11

| Antibody | C27-7-33 | C27-47-56 | Dupilumab |
|---|---|---|---|
| IC50(nM) | 0.03111 | 0.05092 | 0.2533 |

As shown in FIG. 3B and Table 12, the lead-optimized antibodies (reformatted as human IgG4) C27-7-33 and C27-47-56 were tested for their abilities to inhibit IL-13 dependent TF-1 cell proliferation, the lead-optimized antibodies showed improved ability to inhibit TF-1 cell proliferation when compared to the reference antibody Dupilumab.

TABLE 12

| Antibody | C27-7-33 | C27-47-56 | Dupilumab |
|---|---|---|---|
| IC50(nM) | 0.09023 | 0.08492 | 0.3009 |

### Example 7: Inhibition of TARC Release in Human PBMCs Assay

Thymus and activation-regulated chemokine (TARC) release assay: TARC acts on the chemokine receptor CCR4, which is expressed on PBMCs (peripheral blood mononuclear cells) and human T-cell lines. IL-4 and IL-13 are important in promoting the release of TARC (Faffe D S, et al. IL-13 and IL-4 promote TARC release in human airway smooth muscle cells: role of IL-4 receptor genotype. Am J Physiol Lung Cell Mol Physiol. 2003 Oct; 285(4):L907-14). The ability of the anti-IL-4Rα antibodies to inhibit the release of TARC induced by IL-4 or IL-13 was tested, Dupilumab was used as a control. Briefly, PBMCs were isolated from human peripheral blood as described previously (Morris et al., J. Biol. Chem. 274:418-423, 1999) and plated in 96-well plates at 2×10⁵ cells/160 μL/well. Anti-IL-4Rα antibodies were serially diluted at a 1:10 ratio, from a starting concentration of 666.7 nM to 6.67E-05 nM, 204, of the anti-IL-4Rα antibody serial dilutions were added to each well along with human IL-4 with the final concentration of 32 ng/mL or human IL-13 with the final concentration of 200 ng/mL, and the plates were cultured at 37° C., 5% CO₂, for 48 hours. The supernatant of cell culture was collected, and TARC concentration was analyzed using the R&D human CCL17/TARC Quantikine™ ELISA kit according to the manufacturer's instructions. The IC50 values were calculated using GraphPad Prism 5 software (GraphPad Software).

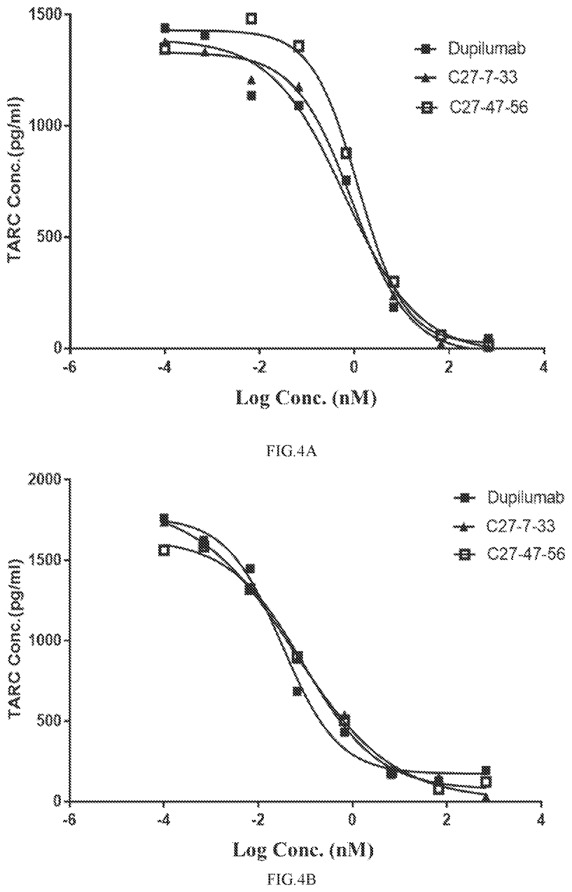

As shown in FIG. 4A and Table 13, the lead-optimized antibodies (reformatted as human IgG4) C27-7-33 and C27-47-56 were tested for their ability to inhibit TARC release induced by hIL-4, all the lead-optimized antibodies showed good efficacy in inhibiting TARC release in PBMCs.

TABLE 13

| Antibody | C27-7-33 | C27-47-56 | Dupilumab |
|---|---|---|---|
| IC50(nM) | 0.9162 | 1.201 | 0.6301 |

As shown in FIG. 4B and Table 14, the lead-optimized antibodies (reformatted as human IgG4) C27-7-33 and C27-47-56 were tested for their abilities to inhibit TARC release induced by hIL-13, all the lead-optimized antibodies showed good efficacy in inhibiting TARC release in PBMCs.

TABLE 14

| Antibody | C27-7-33 | C27-47-56 | Dupilumab |
|---|---|---|---|
| IC50(nM) | 0.05758 | 0.09133 | 0.03228 |

### Example 8: Inhibition of IL-4 or IL-13 Dependent CD23 Upregulation on B Cells in Human PBMCs

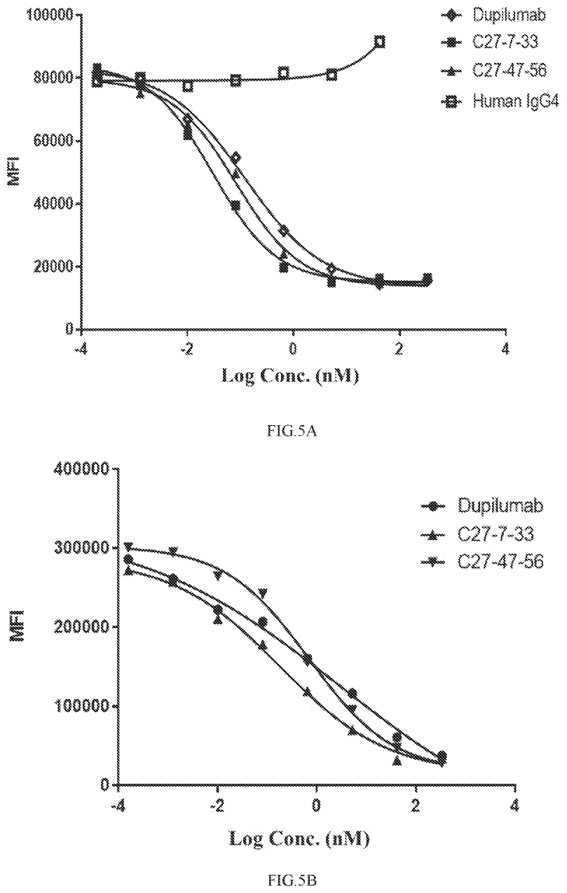

CD23 upregulation assay: This assay is based on the ability of both IL-4 and IL-13 to enhance the expression of the activation-associated surface antigen CD23 on human B cells in PBMCs. The anti-IL-4Rα antibodies were tested for the ability to inhibit CD23 expression induced by IL-4 or IL-13. Dupilumab was used as a control. Briefly, PBMCs were isolated from human peripheral blood and plated in 6-well plates at 1×10⁶ cells/600 μL/well. Anti-IL-4Rα antibodies were serially diluted at a 1:8 ratio, from a starting concentration of 333 nM to 1.59E-04 nM, 2004, of the diluted antibodies were added to the cells, along with human IL-4 with the final concentration of 1 ng/mL or human IL-13 with the final concentration of 20 ng/mL, and cultured at 37° C., 5% CO₂, for 48 hours. Subsequently, PBMCs were co-stained with APC labeled anti-CD19 antibody (to identify B cells) and FITC labeled anti-CD23 antibody (to identify the CD23 expression). After incubation at 4° C. for 30 minutes protected from light, the cells were subjected to FACS analysis, the FITC (FL1) and APC(FL4) were analyzed. The CD23 expression level on B cell was determined by the Mean Fluorescence Intensity (MEI). The IC50 values were calculated using GraphPad Prism 5 software (GraphPad Software). Human IgG4, kappa Isotype Control (Cat #HG4K, Sino Biological Inc.) was used as a negative control, abbreviated as Human IgG4 in FIG. 5A.

As shown in FIG. 5A, and Table 15, the lead-optimized antibodies (reformatted as human IgG4) C27-7-33 and C27-47-56 were tested for their ability to inhibit hIL-4 dependent CD23 upregulation on B cells in PBMCs, and the lead-optimized antibodies showed better efficacy when compared to the reference antibody Dupilumab.

TABLE 15

| Antibody | C27-7-33 | C27-47-56 | Dupilumab |
|---|---|---|---|
| IC50(nM) | 0.03032 | 0.08138 | 0.1194 |

As shown in FIG. 5B, and Table 16, the lead-optimized antibodies (reformatted as human IgG4) C27-7-33 and C27-47-56 were tested for their ability to inhibit hIL-13 dependent CD23 upregulation on B cells in PBMCs, and the lead-optimized antibodies showed better when compared to the reference antibody Dupilumab.

TABLE 16

| Antibody | C27-7-33 | C27-47-56 | Dupilumab |
|---|---|---|---|
| IC50(nM) | 0.1794 | 0.6981 | 2.796 |

1. An isolated anti-IL-4Ra antibody, wherein the anti-IL-4Ra antibody comprises a heavy chain variable domain (V_{H}) comprising:
a heavy chain complementarity determining region (HC-CDR) 1 comprising SYAMH (SEQ ID NO: 1);
an HC-CDR2 comprising GISX₁X₂X₃X₄STYYANSVKG (SEQ ID NO: 78), wherein X₁ is P, S, H, G, or Y, X₂ is S, T, or N, X₃ is G or S, X₄ is S, V, G, T, A, or N; and
an HC-CDR3 comprising X₁X₂X₃X₄YRGGMDV (SEQ ID NO: 79), wherein X₁ is V or S, X₂ is K, F, or R, X₃ is P, V, G, R, S, or L, X₄ is G, A, R, K, or L;
and a light chain variable domain (V_{L}) comprising:
a light chain complementarity determining region (LC-CDR) 1 comprising RASQX₁X₂SX₃AYLA (SEQ ID NO: 80), wherein X₁ is G, S, N, or D, X₂ is I, V, or A, X₃ is S, T, or N;
an LC-CDR2 comprising GTSRRAT (SEQ ID NO: 41); and
an LC-CDR3 comprising QLYGX₁X₂SVT (SEQ ID NO: 81), wherein X₁ is A, S, T, or R, X₂ is T or S.

2. The isolated anti-IL-4Ra antibody of claim 1, comprising:
a V_{H} comprising an HC-CDR1 comprising the amino acid sequence of SEQ ID NO: 1, or a variant thereof comprising up to about 3 amino acid substitutions; an HC-CDR2 comprising the amino acid sequence of any one of SEQ ID NOs: 2-16, or a variant thereof comprising up to about 3 amino acid substitutions; and an HC-CDR3 comprising the amino acid sequence of any one of SEQ ID NOs: 17-30, or a variant thereof comprising up to about 3 amino acid substitutions; and
a V_{L} comprising an LC-CDR1 comprising the amino acid sequence of any one of SEQ ID NOs: 31-40, or a variant thereof comprising up to about 3 amino acid substitutions;
an LC-CDR2 comprising the amino acid sequence of SEQ ID NO: 41, or a variant thereof comprising up to about 3 amino acid substitutions; and an LC-CDR3 comprising the amino acid sequence of any one of SEQ ID NOs: 42-47, or a variant thereof comprising up to about 3 amino acid substitutions.

3. The isolated anti-IL-4Ra antibody of claim 1, comprising a V_{H} comprising an HC-CDR1, an HC-CDR2, and an HC-CDR3 of a V_{H} comprising the amino acid sequence of any one of SEQ ID NOs: 48-64; and a V_{L} comprising an LC-CDR1, an LC-CDR2, and an LC-CDR3 of a V_{L} comprising the amino acid sequence of any one of SEQ ID NOs: 65-77.

4. The isolated anti-IL-4Ra antibody of claim 3, comprising:
(i) a V_{H} comprising an HC-CDR1, an HC-CDR2, and an HC-CDR3 of a V_{H} comprising the amino acid sequence of SEQ ID NO: 48; and a V_{L} comprising an LC-CDR1, an LC-CDR2, and an LC-CDR3 of a V_{L} comprising the amino acid sequence of SEQ ID NO: 65;
(ii) a V_{H} comprising an HC-CDR1, an HC-CDR2, and an HC-CDR3 of a V_{H} comprising the amino acid sequence of SEQ ID NO: 49; and a V_{L} comprising an LC-CDR1, an LC-CDR2, and an LC-CDR3 of a V_{L} comprising the amino acid sequence of SEQ ID NO: 66;
(iii) a V_{H} comprising an HC-CDR1, an HC-CDR2, and an HC-CDR3 of a V_{H} comprising the amino acid sequence of SEQ ID NO: 50, and a V_{L} comprising an LC-CDR1, an LC-CDR2, and an LC-CDR3 of a V_{L} comprising the amino acid sequence of SEQ ID NO: 66;
(iv) a V_{H} comprising an HC-CDR1, an HC-CDR2, and an HC-CDR3 of a V_{H} comprising the amino acid sequence of SEQ ID NO: 51; and a V_{L} comprising an LC-CDR1, an LC-CDR2, and an LC-CDR3 of a V_{L} comprising the amino acid sequence of SEQ ID NO: 67;
(v) a V_{H} comprising an HC-CDR1, an HC-CDR2, and an HC-CDR3 of a V_{H} comprising the amino acid sequence of SEQ ID NO: 52; and a V_{L} comprising an LC-CDR1, an LC-CDR2, and an LC-CDR3 of a V_{L} comprising the amino acid sequence of SEQ ID NO: 67;
(vi) a Vii comprising an HC-CDR1, an HC-CDR2, and an HC-CDR3 of a V_{H} comprising the amino acid sequence of SEQ ID NO: 53; and a V_{L} comprising an LC-CDR1, an LC-CDR2, and an LC-CDR3 of a V_{L} comprising the amino acid sequence of SEQ ID NO: 66;
(vii) a V_{H} comprising an HC-CDR1, an HC-CDR2, and an HC-CDR3 of a V_{H} comprising the amino acid sequence of SEQ ID NO: 54; and a V_{L} comprising an LC-CDR1, an LC-CDR2, and an LC-CDR3 of a V_{L} comprising the amino acid sequence of SEQ ID NO: 67;
(viii) a V_{H} comprising an HC-CDR1, an HC-CDR2, and an HC-CDR3 of a V_{H} comprising the amino acid sequence of SEQ ID NO: 55; and a V_{L} comprising an LC-CDR1, an LC-CDR2, and an LC-CDR3 of a V_{L} comprising the amino acid sequence of SEQ ID NO: 68;
(ix) a Vii comprising an HC-CDR1, an HC-CDR2, and an HC-CDR3 of a V_{H} comprising the amino acid sequence of SEQ ID NO: 56; and a V_{L} comprising an LC-CDR1, an LC-CDR2, and an LC-CDR3 of a V_{L} comprising the amino acid sequence of SEQ ID NO: 69;
(x) a V_{H} comprising an HC-CDR1, an HC-CDR2, and an HC-CDR3 of a V_{H} comprising the amino acid sequence of SEQ ID NO: 57; and a V_{L} comprising an LC-CDR1, an LC-CDR2, and an LC-CDR3 of a V_{L} comprising the amino acid sequence of SEQ ID NO: 70;
(xi) a V_{H} comprising an HC-CDR1, an HC-CDR2, and an HC-CDR3 of a V_{H} comprising the amino acid sequence of SEQ ID NO: 50; and a V_{L} comprising an LC-CDR1, an LC-CDR2, and an LC-CDR3 of a V_{L} comprising the amino acid sequence of SEQ ID NO: 71;
(xii) a V_{H} comprising an HC-CDR1, an HC-CDR2, and an HC-CDR3 of a V_{H} comprising the amino acid sequence of SEQ ID NO: 58; and a V_{L} comprising an LC-CDR1, an LC-CDR2, and an LC-CDR3 of a V_{L} comprising the amino acid sequence of SEQ ID NO: 72;
(xiii) a V_{H} comprising an HC-CDR1, an HC-CDR2, and an HC-CDR3 of a V_{H} comprising the amino acid sequence of SEQ ID NO: 59; and a V_{L} comprising an LC-CDR1, an LC-CDR2, and an LC-CDR3 of a V_{L} comprising the amino acid sequence of SEQ ID NO: 73,
(xiv) a V_{H} comprising an HC-CDR1, an HC-CDR2, and an HC-CDR3 of a V_{H} comprising the amino acid sequence of SEQ ID NO: 60, and a V_{L} comprising an LC-CDR1, an LC-CDR2, and an LC-CDR3 of a V_{L} comprising the amino acid sequence of SEQ ID NO: 74;
(xv) a V_{H} comprising an HC-CDR1, an HC-CDR2, and an HC-CDR3 of a V_{H} comprising the amino acid sequence of SEQ ID NO: 61; and a V_{L} comprising an LC-CDR1, an LC-CDR2, and an LC-CDR3 of a V_{L} comprising the amino acid sequence of SEQ ID NO: 75;
(xvi) a V_{H} comprising an HC-CDR1, an HC-CDR2, and an HC-CDR3 of a V_{H} comprising the amino acid sequence of SEQ ID NO: 62; and a V_{L} comprising an LC-CDR1, an LC-CDR2, and an LC-CDR3 of a V_{L} comprising the amino acid sequence of SEQ ID NO: 69;
(xvii) a V_{H} comprising an HC-CDR1, an HC-CDR2, and an HC-CDR3 of a V_{H} comprising the amino acid sequence of SEQ ID NO: 63; and a V_{L} comprising an LC-CDR1, an LC-CDR2, and an LC-CDR3 of a V_{L} comprising the amino acid sequence of SEQ ID NO: 76; or
(xviii) a V_{H} comprising an HC-CDR1, an HC-CDR2, and an HC-CDR3 of a V_{H} comprising the amino acid sequence of SEQ ID NO: 64; and a V_{L} comprising an LC-CDR1, an LC-CDR2, and an LC-CDR3 of a V_{L} comprising the amino acid sequence of SEQ ID NO: 77.

5. (canceled)

6. The isolated anti-IL-4Ra antibody of claim 1, comprising:
(i) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 17, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs;
(ii) a V_{H} comprising an HC-CDR1 comprising the amino acid sequence of SEQ ID NO: 1, an HC-CDR2 comprising the amino acid sequence of SEQ ID NO: 3, and an HC-CDR3 comprising the amino acid sequence of SEQ ID NO: 18, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(iii) a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(iv) a V_{H} comprising an HC-CDR1 comprising the amino acid sequence of SEQ ID NO: 1, an HC-CDR2 comprising the amino acid sequence of SEQ ID NO: 5, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs;
(v) a V_{H} comprising an HC-CDR1 comprising the amino acid sequence of SEQ ID NO: 1, an HC-CDR2 comprising the amino acid sequence of SEQ ID NO: 6, and an HC-CDR3 comprising the amino acid sequence of SEQ ID NO: 21, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs;
(vi) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 22, or a variant thereof comprising up to about 5 amino acid substitutions in the HC-CDRs; and a V_{L} comprising an LC-CDR1 comprising the amino acid sequence of SEQ ID NO: 32, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(vii) a V_{H} comprising an HC-CDR1 comprising the amino acid sequence of SEQ ID NO: 1, an HC-CDR2 comprising the amino acid sequence of SEQ ID NO: 8, and an HC-CDR3 comprising the amino acid sequence of SEQ ID NO: 23, or a variant thereof comprising up to about 5 amino acid substitutions in the HC-CDRs; and a V_{L} comprising an LC-CDR1 comprising the amino acid sequence of SEQ ID NO: 31, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs;
(viii) a V_{H} comprising an HC-CDR1 comprising the amino acid sequence of SEQ ID NO: 1, an HC-CDR2 comprising the amino acid sequence of SEQ ID NO: 9, and an HC-CDR3 comprising the amino acid sequence of SEQ ID NO: 24, or a variant thereof comprising up to about 5 amino acid substitutions in the HC-CDRs; and a V_{L} comprising an LC-CDR1 comprising the amino acid sequence of SEQ ID NO: 33, an LC-CDR2 comprising the amino acid sequence of SEQ ID NO: 41, and an LC-CDR3 comprising the amino acid sequence of SEQ ID NO: 44, or a variant thereof comprising up to about 5 amino acid substitutions in the LC-CDRs;
(ix) a V_{H} comprising an HC-CDR1 comprising the amino acid sequence of SEQ ID NO: 1, an HC-CDR2 comprising the amino acid sequence of SEQ ID NO: 10, and an HC-CDR3 comprising the amino acid sequence of SEQ ID NO: 25, or a variant thereof comprising up to about 5 amino acid substitutions in the HC-CDRs; and a V_{L} comprising an LC-CDR1 comprising the amino acid sequence of SEQ ID NO. 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(x) a V_{H} comprising an HC-CDR1 comprising the amino acid sequence of SEQ ID NO: 1, an HC-CDR2 comprising the amino acid sequence of SEQ ID NO: 11, and an HC-CDR3 comprising the amino acid sequence of SEQ ID NO: 26, or a variant thereof comprising up to about 5 amino acid substitutions in the HC-CDRs; and a V_{L} comprising an LC-CDR1 comprising the amino acid sequence of SEQ ID NO: 35, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(xi) a V_{H} comprising an HC-CDR1 comprising the amino acid sequence of SEQ ID NO: 1, an HC-CDR2 comprising the amino acid sequence of SEQ ID NO: 4, and an HC-CDR3 comprising the amino acid sequence of SEQ ID NO: 19, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(xii) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 27, or a variant thereof comprising up to about 5 amino acid substitutions in the HC-CDRs; and a V_{L} comprising an LC-CDR1 comprising the amino acid sequence of SEQ ID NO: 37, an LC-CDR2 comprising the amino acid sequence of SEQ ID NO: 41, and an LC-CDR3 comprising the amino acid sequence of SEQ ID NO: 42, or a variant thereof comprising up to about 5 amino acid substitutions in the LC-CDRs;
(xiii) a V_{H} comprising an HC-CDR1 comprising the amino acid sequence of SEQ ID NO: 1, an HC-CDR2 comprising the amino acid sequence of SEQ ID NO: 12, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 45, or a variant thereof comprising up to about 5 amino acid substitutions in the LC-CDRs;
(xiv) a V_{H} comprising an HC-CDR1 comprising the amino acid sequence of SEQ ID NO: 1, an HC-CDR2 comprising the amino acid sequence of SEQ ID NO: 13, and an HC-CDR3 comprising the amino acid sequence of SEQ ID NO: 28, or a variant thereof comprising up to about 5 amino acid substitutions in the HC-CDRs; and a V_{L} comprising an LC-CDR1 comprising the amino acid sequence of SEQ ID NO: 36, an LC-CDR2 comprising the amino acid sequence of SEQ ID NO: 41, and an LC-CDR3 comprising the amino acid sequence of SEQ ID NO: 46, or a variant thereof comprising up to about 5 amino acid substitutions in the LC-CDRs;
(xv) a V_{H} comprising an HC-CDR1 comprising the amino acid sequence of SEQ ID NO: 1, an HC-CDR2 comprising the amino acid sequence of SEQ ID NO: 14, and an HC-CDR3 comprising the amino acid sequence of SEQ ID NO: 29, or a variant thereof comprising up to about 5 amino acid substitutions in the HC-CDRs; and a V_{L} comprising an LC-CDR1 comprising the amino acid sequence of SEQ ID NO. 39, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(xvi) a V_{H} comprising an HC-CDR1 comprising the amino acid sequence of SEQ ID NO: 1, an HC-CDR2 comprising the amino acid sequence of SEQ ID NO: 15, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 34, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs;
(xvii) a V_{H} comprising an HC-CDR1 comprising the amino acid sequence of SEQ ID NO: 1, an HC-CDR2 comprising the amino acid sequence of SEQ ID NO: 7, and an HC-CDR3 comprising the amino acid sequence of SEQ ID NO: 30, or a variant thereof comprising up to about 5 amino acid substitutions in the HC-CDRs; and a V_{L} comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 43, or a variant thereof comprising up to about 5 amino acid substitutions in the LC-CDRs; or
(xviii) a V_{H} comprising an HC-CDR1 comprising the amino acid sequence of SEQ ID NO: 1, an HC-CDR2 comprising the amino acid sequence of SEQ ID NO: 16, and an HC-CDR3 comprising the amino acid sequence of SEQ ID NO: 20, or a variant thereof comprising up to about 5 amino acid substitutions in the HC-CDRs; and a V_{L} comprising an LC-CDR1 comprising the amino acid sequence of SEQ ID NO: 38, an LC-CDR2 comprising the amino acid sequence of SEQ ID NO: 41, and an LC-CDR3 comprising the amino acid sequence of SEQ ID NO: 47, or a variant thereof comprising up to about 5 amino acid substitutions in the LC-CDRs.

7. The isolated anti-IL-4Ra antibody of claim 1, comprising:
a V_{H} comprising the amino acid sequence of any one of SEQ ID NOs: 48-64, or a variant thereof having at least about 90% sequence identity to the amino acid sequence of any one of SEQ ID NOs: 48-64; and
a V_{L} comprising the amino acid sequence of any one of SEQ ID NOs: 65-77, or a variant thereof having at least about 90% sequence identity to the amino acid sequence of any one of SEQ ID NOs: 65-77.

8. The isolated anti-IL-4Ra antibody of claim 7, comprising:
(i) a V_{H} comprising the amino acid sequence of SEQ ID NO: 48, or a variant thereof having at least about 90% sequence identity to the amino acid sequence of SEQ ID NO: 48; and a V_{L} comprising the amino acid sequence of SEQ ID NO: 65, or a variant thereof having at least about 90% sequence identity to the amino acid sequence of SEQ ID NO: 65;
(ii) a V_{H} comprising the amino acid sequence of SEQ ID NO: 49, or a variant thereof having at least about 90% sequence identity to the amino acid sequence of SEQ ID NO: 49; and
a V_{L} comprising the amino acid sequence of SEQ ID NO: 66, or a variant thereof having at least about 90% sequence identity to the amino acid sequence of SEQ ID NO: 66;
(iii) a V_{H} comprising the amino acid sequence of SEQ ID NO: 50, or a variant thereof having at least about 90% sequence identity to the amino acid sequence of SEQ ID NO: 50; and
a V_{L} comprising the amino acid sequence of SEQ ID NO: 66, or a variant thereof having at least about 90% sequence identity to the amino acid sequence of SEQ ID NO: 66;
(iv) a V_{H} comprising the amino acid sequence of SEQ ID NO: 51, or a variant thereof having at least about 90% sequence identity to the amino acid sequence of SEQ ID NO: 51; and
a V_{L} comprising the amino acid sequence of SEQ ID NO: 67, or a variant thereof having at least about 90% sequence identity to the amino acid sequence of SEQ ID NO: 67;
(v) a V_{H} comprising the amino acid sequence of SEQ ID NO: 52, or a variant thereof having at least about 90% sequence identity to the amino acid sequence of SEQ ID NO: 52; and a V_{L} comprising the amino acid sequence of SEQ ID NO: 67, or a variant thereof having at least about 90% sequence identity to the amino acid sequence of SEQ ID NO: 67;
(vi) a V_{H} comprising the amino acid sequence of SEQ ID NO: 53, or a variant thereof having at least about 90% sequence identity to the amino acid sequence of SEQ ID NO: 53; and a V_{L} comprising the amino acid sequence of SEQ ID NO: 66, or a variant thereof having at least about 90% sequence identity to the amino acid sequence of SEQ ID NO: 66;
(vii) a V_{H} comprising the amino acid sequence of SEQ ID NO: 54, or a variant thereof having at least about 90% sequence identity to the amino acid sequence of SEQ ID NO: 54; and a V_{L} comprising the amino acid sequence of SEQ ID NO: 67, or a variant thereof having at least about 90% sequence identity to the amino acid sequence of SEQ ID NO: 67;
(viii) a V_{H} comprising the amino acid sequence of SEQ ID NO: 55, or a variant thereof having at least about 90% sequence identity to the amino acid sequence of SEQ ID NO: 55; and a V_{L} comprising the amino acid sequence of SEQ ID NO: 68, or a variant thereof having at least about 90% sequence identity to the amino acid sequence of SEQ ID NO: 68;
(ix) a V_{H} comprising the amino acid sequence of SEQ ID NO: 56, or a variant thereof having at least about 90% sequence identity to the amino acid sequence of SEQ ID NO: 56; and a V_{L} comprising the amino acid sequence of SEQ ID NO: 69, or a variant thereof having at least about 90% sequence identity to the amino acid sequence of SEQ ID NO: 69;
(x) a V_{H} comprising the amino acid sequence of SEQ ID NO: 57, or a variant thereof having at least about 90% sequence identity to the amino acid sequence of SEQ ID NO: 57; and a V_{L} comprising the amino acid sequence of SEQ ID NO: 70, or a variant thereof having at least about 90% sequence identity to the amino acid sequence of SEQ ID NO: 70;
(xi) a V_{H} comprising the amino acid sequence of SEQ ID NO: 50, or a variant thereof having at least about 90% sequence identity to the amino acid sequence of SEQ ID NO: 50; and
a V_{L} comprising the amino acid sequence of SEQ ID NO: 71, or a variant thereof having at least about 90% sequence identity to the amino acid sequence of SEQ ID NO: 71;
(xii) a V_{H} comprising the amino acid sequence of SEQ ID NO: 58, or a variant thereof having at least about 90% sequence identity to the amino acid sequence of SEQ ID NO: 58; and a V_{L} comprising the amino acid sequence of SEQ ID NO: 72, or a variant thereof having at least about 90% sequence identity to the amino acid sequence of SEQ ID NO: 72;
(xiii) a V_{H} comprising the amino acid sequence of SEQ ID NO: 59, or a variant thereof having at least about 90% sequence identity to the amino acid sequence of SEQ ID NO: 59; and a V_{L} comprising the amino acid sequence of SEQ ID NO: 73, or a variant thereof having at least about 90% sequence identity to the amino acid sequence of SEQ ID NO: 73;
(xiv) a V_{H} comprising the amino acid sequence of SEQ ID NO: 60, or a variant thereof having at least about 90% sequence identity to the amino acid sequence of SEQ ID NO: 60; and a V_{L} comprising the amino acid sequence of SEQ ID NO: 74, or a variant thereof having at least about 90% sequence identity to the amino acid sequence of SEQ ID NO: 74;
(xv) a V_{H} comprising the amino acid sequence of SEQ ID NO: 61, or a variant thereof having at least about 90% sequence identity to the amino acid sequence of SEQ ID NO: 61; and a V_{L} comprising the amino acid sequence of SEQ ID NO: 75, or a variant thereof having at least about 90% sequence identity to the amino acid sequence of SEQ ID NO: 75;
(xvi) a V_{H} comprising the amino acid sequence of SEQ ID NO: 62, or a variant thereof having at least about 90% sequence identity to the amino acid sequence of SEQ ID NO: 62; and a V_{L} comprising the amino acid sequence of SEQ ID NO: 69, or a variant thereof having at least about 90% sequence identity to the amino acid sequence of SEQ ID NO: 69;
(xvii) a V_{H} comprising the amino acid sequence of SEQ ID NO: 63, or a variant thereof having at least about 90% sequence identity to the amino acid sequence of SEQ ID NO: 63; and a V_{L} comprising the amino acid sequence of SEQ ID NO: 76, or a variant thereof having at least about 90% sequence identity to the amino acid sequence of SEQ ID NO: 76; or
(xviii) a V_{H} comprising the amino acid sequence of SEQ ID NO: 64, or a variant thereof having at least about 90% sequence identity to the amino acid sequence of SEQ ID NO: 64; and a V_{L} comprising the amino acid sequence of SEQ ID NO: 77, or a variant thereof having at least about 90% sequence identity to the amino acid sequence of SEQ ID NO: 77.

9. An isolated anti-IL-4Ra antibody that specifically binds to IL-4Ra competitively with the isolated anti-IL-4Ra antibody of claim 1, or specifically binds to the same epitope as the isolated anti-IL-4Ra antibody of claim 1.

10. The isolated anti-IL-4Rα antibody according to claim 1, wherein: (i) the anti-IL-4Rα antibody comprises an Fc fragment;
(ii) the anti-IL-4Rα antibody is a full-length IgA, IgD, IgE, IgG or IgM antibody:
(iii) the anti-IL-4Rα antibody is a full-length IgG1, IgG2, IgG3 or IgG4 antibody;
(iv) the anti-IL-4Rα antibody is chimeric, human, or humanized;
(v) the anti-IL-4Rα antibody is an antigen binding fragment selected from the group consisting of a Fab, a Fab′, a F(ab)′2, a Fab′-SH, a single-chain Fv (scFv), an Fv fragment, a dAb, a Fd, a nanobody, a diabody, and a linear antibody; or
(vi) the anti-IL-4Rα antibody binds to the human IL-4Rα with a Kd from about 0.1 pM to about 10 nM.

11-14. (canceled)

15. The isolated anti-IL-4Rα antibody of claim 1, wherein the anti-IL-4Rα antibody inhibits binding of IL-4 to IL-4Ra, and wherein the anti-IL-4Rα antibody:
(i) has an IC50 neutralizing potency of 18 nM or less in a hIL-4R-mediated cellular function inhibition assay in vitro with 1.2 ng/ml of human IL-4;
(ii) has an IC50 neutralizing potency of 2.0 nM or less in a hIL-4R-mediated cellular function inhibition assay in vitro with 4 ng/ml of human IL-13;
(iii) has an IC50 neutralizing potency of 0.8 nM or less in a TF-1 cell proliferation assay with 2 ng/ml of human IL4;
(iv) has an IC50 neutralizing potency of 0.9 nM or less in a TF-1 cell proliferation assay with 10 ng/ml of human IL-13;
(v) has an IC50 neutralizing potency of 1.9 nM or less in a thymus and activation-regulated chemokine (TARC) release assay with 32 ng/ml of human IL-4;
(vi) has an IC50 neutralizing potency of 0.1 nM or less in a thymus and activation-regulated chemokine (TARC) release assay with 200 ng/ml of human IL-13;
(vii) has an IC50 neutralizing potency of 0.4 nM or less in a CD23 upregulation assay with 1 ng/ml of human IL-4; or
(viii) has an IC50 neutralizing potency of 8.4 nM or less in a CD23 upregulation assay with 20 ng/ml of human IL-13.

16. An isolated nucleic acid molecule that encodes the anti-IL-4Rα antibody according to claim 1.

17. A vector comprising the nucleic acid molecule of claim 16.

18. An isolated host cell comprising the nucleic acid of claim 16.

19. A method of producing an anti-IL-4Rα antibody, comprising:
a) culturing the host cell of claim 18 under conditions effective to express the anti-IL-4Rα antibody; and
b) obtaining the expressed anti-IL-4Rα antibody from the host cell.

20. A pharmaceutical composition comprising the anti-IL-4Rα antibody according to claim 1, and a pharmaceutically acceptable carrier.

21. A method of treating a disease or condition in an individual in need thereof, comprising administering to the individual an effective amount of the pharmaceutical composition of claim 20.

22. The method of claim 21, wherein the disease or condition is caused by increased expression, activity or sensitivity of human interleukin-4 (hIL-4) and/or human interleukin-13 (hIL-13) and/or human interleukin-4 receptor alpha (hIL-4Ra).

23. The method of claim 22, wherein the disease or condition is selected from the group consisting of asthma, atopic dermatitis, arthritis, dermatitis herpetiformis, chronic idiopathic urticaria, scleroderma, hypertrophic scarring, Whipple's Disease, benign prostate hyperplasia, lung disorders, inflammatory disorders, allergic reactions, Kawasaki disease, sickle cell disease, Churg Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, and nephrosis.
